(19) United States
(12) Patent Application Publication
(10) Pub. No.: US 2024/0317707 A1
(43) Pub. Date: Sep. 26, 2024

(54) COVALENT AND NON-COVALENT INHIBITORS OF CORONAVIRUS PAPAIN-LIKE PROTEASE

(71) Applicants: UT-Battelle, LLC, Oak Ridge, TN (US); Northeastern University, Boston, MA (US)

(72) Inventors: Jerry M. Parks, Knoxville, TN (US); Brian Sanders, Knoxville, TN (US); Lori Ferrins, Roslindale, MA (US)

(21) Appl. No.: 18/613,350

(22) Filed: Mar. 22, 2024

Related U.S. Application Data
(60) Provisional application No. 63/454,205, filed on Mar. 23, 2023.

(51) Int. Cl. C07D 401/06; A61K 31/192; A61K 31/235; A61K 31/337; A61K 31/427; A61P 31/14; C07C 241/04; C07D 305/08; C07D 409/06; C07D 417/06; C12N 9/99

(52) U.S. Cl. CPC C07D 401/06; A61K 31/192; A61K 31/235; A61K 31/337; A61K 31/427; A61P 31/14; C07C 241/04; C07D 305/08; C07D 409/06; C07D 417/06; C12N 9/99; C07C 2602/10

(57) ABSTRACT

A compound having the following structure:

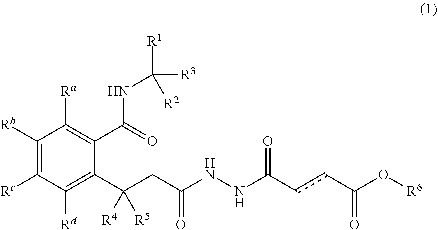

R¹ is a hydrocarbon group containing at least one aromatic or heteroaromatic ring or fused ring system, wherein the at least one aromatic or heteroaromatic ring or fused ring system is optionally substituted; R² and R³ are independently selected from the group consisting of hydrogen (H), halogen atom, and hydrocarbon groups containing 1-6 carbon atoms and optionally substituted; R⁴ and R⁵ are independently selected from H, halogen atom, and hydrocarbon groups containing 1-6 carbon atoms and optionally substituted; R⁶ is H or a hydrocarbon group containing 1-12 carbon atoms and optionally containing one or more heteroatoms selected from O, N, S, and F; and R^{a}, R^{b}, R^{c}, and R^{d} are independently selected from H; halogen atom; hydrocarbon groups containing 1-3 carbon atoms; amine groups; amide groups; and —(CH₂)ₚ—T groups, wherein T is a hydrocarbon group with —NH— linkage; and pharmaceutically acceptable salts thereof.

### CROSS REFERENCE TO RELATED APPLICATION

The present application claims benefit of U.S. Provisional Application No. 63/454,205 filed on Mar. 23, 2023, all of the contents of which are incorporated herein by reference.

### STATEMENT REGARDING FEDERALLY SPONSORED RESEARCH

This invention was made with government support under Prime Contract No. DE-AC05-00OR22725 awarded by the U.S. Department of Energy, and Grant Nos. 2R01AI114685, 4R33AI141227, and 5R01AI148235 awarded by the National Institutes of Health (NIH). The government has certain rights in the invention.

### FIELD OF THE INVENTION

The present invention generally relates to inhibitors of coronavirus papain-like protease, and more particularly, wherein the inhibitors are N-aryl benzamide compounds. The present invention also relates to the use of such inhibitors for inhibiting papain-like protease activity in a subject, or more particularly, treating coronavirus infection in a subject.

### BACKGROUND

Severe acute respiratory syndrome coronavirus 2 (SARS-CoV-2) causes COVID-19, which has led to a global pandemic. The virus is highly transmissible and leads to severe, and in many cases life-threatening, respiratory disease with an overall case fatality rate of 2.1% as of August 2021. Few effective drugs have been developed to date, with molnupiravir, nirmatrelvir, and ritonavir being the only currently available oral antivirals for treating SARS-CoV-2 infections. Although recently developed vaccines can be highly effective in preventing COVID-19 or reducing its severity, the emergence of variant strains limits their effectiveness. In addition, vaccines are not yet widely available worldwide, many are unwilling to receive them, and others, despite having received the vaccine, may still contract the disease in breakthrough COVID cases. Efforts to repurpose existing drugs have been largely ineffective, and few effective pharmaceutical treatments have been identified to date. Thus, there is an urgent need to develop new direct-acting antiviral therapeutics that are effective against SARS-CoV-2 and future coronaviruses.

### SUMMARY

In a first aspect, the present disclosure is directed to covalent inhibitor molecules of coronavirus papain-like protease (PLpro). The covalent inhibitor molecules are substituted (R)—N-(1-(naphthalen-1-yl)ethyl)benzamide compounds having the following structure:

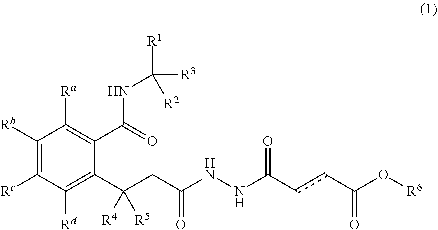

wherein: R¹ is a hydrocarbon group containing at least one aromatic ring or fused ring system; R² and R³ are independently selected from the group consisting of hydrogen (H), halogen atom, and hydrocarbon groups containing 1-6 carbon atoms optionally substituted with one or more halogen atoms and optionally containing an —O— or —NH— linkage, wherein R² and R³ are optionally interconnected to form a three-, four-, five-, or six-membered cycloalkyl or heterocycloalkyl ring; R⁴ and R⁵ are independently selected from the group consisting of H, halogen atom, and hydrocarbon groups containing 1-6 carbon atoms optionally substituted with one or more halogen atoms and optionally containing an —O— or —NH— linkage, wherein R⁴ and R⁵ are optionally interconnected to form a three-, four-, five-, or six-membered cycloalkyl or heterocycloalkyl ring; R⁶ is H or a hydrocarbon group containing 1-12 carbon atoms and optionally containing one or more heteroatoms selected from O, N, and S; R^{a}, R^{b}, R^{c}, and R^{d} are independently selected from the group consisting of H; halogen atom; hydrocarbon groups containing 1-3 carbon atoms; amine group of the formula NR′₂; amide group of the formula NHC(O)R′; and —(CH₂)ₚ-T, wherein T is a hydrocarbon group containing at least one —NH— linkage; R′ is independently selected from H and hydrocarbon groups containing 1-6 carbon atoms; and p is an integer of 0-3; the dashed line represents an optional double bond (i.e., a single bond or double bond); and pharmaceutically acceptable salts thereof, specific (isolated or enriched) enantiomers thereof, and racemic mixtures thereof.

The covalent inhibitor molecules having the above generic structural Formula (1) include a tail portion containing a linking portion (—C(R⁴R⁵)CH₂C(O)NH—NH—) attached to an electrophilic warhead (—C(O)CH═CHC(O)OR⁶)) to make them potent covalent PLpro inhibitors. The above tail portion provides an enhanced potency in several ways, as follows: (i) it mimics the glycine-glycine linkage of native protein substrates but does not participate in standard amide cleavage chemistry; (ii) it occupies minimal steric space, which permits it to fit into the narrow substrate binding cleft of the PLpro enzyme; (iii) it contains hydrogen bond donors and acceptors that interact with the enzyme; and (iv) it terminates with a reactive electrophilic group, placing it near the catalytic cysteine in the active site, Cys111, and allowing for covalent bond formation. In some embodiments, one or more of R^{a}, R^{b}, R^{c}, and R^{d} (or at least or only R^{a}) is a non-hydrogen functional group (e.g., amine or amide group) that mimics positively-charged arginine or lysine residues of the native substrate sequence and thereby enhances efficacy.

In some embodiments, the covalent inhibitor molecule is an enantiomerically enriched or isolated version having the following formula in which R³ is H and thus not shown:

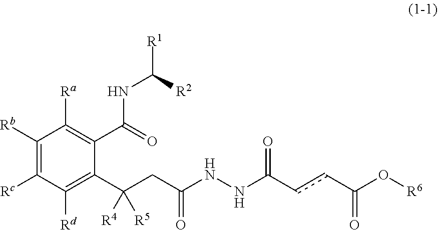

wherein R² is a halogen atom or a hydrocarbon group containing 1-6 carbon atoms optionally substituted with one or more halogen atoms and optionally containing an —O— or —NH— linkage; and R¹, R⁴, R⁵, R⁶, R^{a}, R^{b}, R^{c}, and R^{d} are as defined above.

In some embodiments, the covalent inhibitor molecules have the following more specific structure:

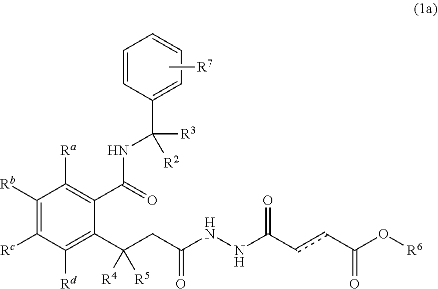

wherein R², R³, R⁴, R⁵, R⁶, R^{a}, R^{b}, R^{c}, and R^{d} are as defined in claim 1; and R⁷ comprises a heteroaryl ring.

In some embodiments, the covalent inhibitor molecule of Formula (Ia) is an enantiomerically enriched or isolated version having the following formula in which R³ is H and thus not shown:

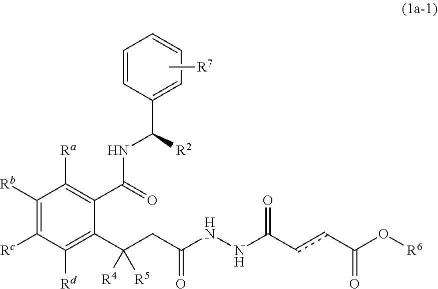

In other embodiments, the covalent inhibitor molecule of Formula (1) has the following structure:

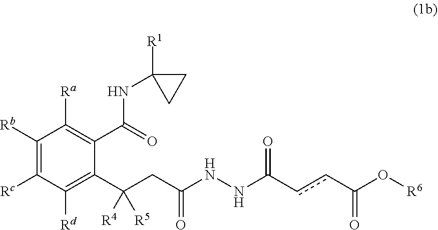

wherein R¹, R⁴, R⁵, R⁶, R^{a}, R^{b}, R^{c}, and R^{d} are as defined above.

In other embodiments, the covalent inhibitor molecule of Formula (1) has the following structure:

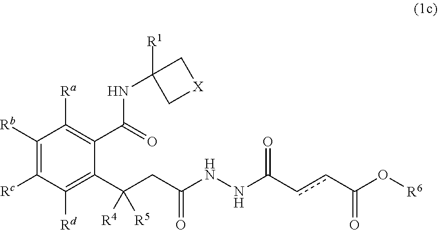

wherein R¹, R⁴, R⁵, R⁶, R^{a}, R^{b}, R^{c}, and R^{d} are as defined above.

In other embodiments, the covalent inhibitor molecule of Formula (1) has the following structure:

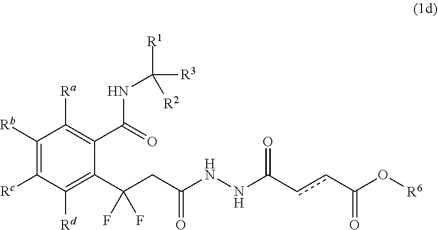

wherein R¹, R², R³, R⁶, R^{a}, R^{b}, R^{c}, and R^{d} are as defined above.

In another aspect, the present disclosure is directed to a method for inhibiting papain-like protease (PLpro) activity in a subject, the method comprising administering a therapeutically effective dosage of a compound of Formula (1) or any sub-formula thereof or particular compound thereof to the subject to result in inhibition of PLpro activity in the subject. In more particular embodiments, the method is directed to treating coronavirus infection in a subject, the method comprising administering a therapeutically effective dosage of a compound of Formula (1) or any sub-formula thereof or particular compound thereof to the subject to result in inhibition or prevention of one or more coronavirus symptoms in the subject.

### DETAILED DESCRIPTION

As used herein, the term “hydrocarbon group” (also denoted by the group R) is defined as a chemical group containing at least carbon and hydrogen atoms. In some embodiments, R is composed solely of carbon and hydrogen. In other embodiments, the hydrocarbon group contains carbon, hydrogen, and optionally, one or more fluorine atoms to result in partial or complete fluorination of the hydrocarbon group. In different embodiments, one or more of the hydrocarbon groups or linkers can contain precisely, or a minimum of, or a maximum of, for example, 1, 2, 3, 4, 5, 6, 7, 8, 9, 10, 12, 15, 18, 20, 22, 24, 26, 28, or 30 carbon atoms, or a number of carbon atoms within a particular range bounded by any two of the foregoing carbon numbers (e.g., 1-30, 1-20, 1-12, 1-8, 1-6, 1-5, 1-4, 1-3, 2-30, 2-20, 2-12, 2-8, 2-6, 2-5, 2-4, or 2-3 carbon atoms). Hydrocarbon groups in different compounds described herein, or in different generic groups of a compound, may possess the same or different number (or preferred range thereof) of carbon atoms. For example, as further discussed below, any one of R¹, R², R³, R⁴, R⁵, R⁶, R^{a}, R^{b}, R^{c}, and R^{d} in any of the generic formulas disclosed herein may independently contain a number of carbon atoms within any of the ranges provided above.

In a first set of embodiments, the hydrocarbon group (R) is a saturated and straight-chained group, i.e., a straight-chained (linear) alkyl group. Some examples of straight-chained alkyl groups include methyl, ethyl, n-propyl, n-butyl, n-pentyl, n-hexyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-eicosyl, n-docosyl, n-tetracosyl, n-hexacosyl, n-octacosyl, and n-triacontyl groups.

In a second set of embodiments, the hydrocarbon group (R) is saturated and branched, i.e., a branched alkyl group. Some examples of branched alkyl groups include isopropyl (2-propyl), isobutyl (2-methylprop-1-yl), sec-butyl (2-butyl), t-butyl (1,1-dimethylethyl-1-yl), 2-pentyl, 3-pentyl, 2-methylbut-1-yl, isopentyl (3-methylbut-1-yl), 1,2-dimethylprop-1-yl, 1,1-dimethylprop-1-yl, neopentyl (2,2-dimethylprop-1-yl), 2-hexyl, 3-hexyl, 2-methylpent-1-yl, 3-methylpent-1-yl, isohexyl (4-methylpent-1-yl), 1,1-dimethylbut-1-yl, 1,2-dimethylbut-1-yl, 2,2-dimethylbut-1-yl, 2,3-dimethylbut-1-yl, 3,3-dimethylbut-1-yl, 1,1,2-trimethylprop-1-yl, and 1,2,2-trimethylprop-1-yl groups, isoheptyl, isooctyl, and the numerous other branched alkyl groups having up to 20 or 30 carbon atoms, wherein the “1-yl” suffix represents the point of attachment of the group.

In a third set of embodiments, the hydrocarbon group (R) is saturated and cyclic, i.e., a cycloalkyl group. Some examples of cycloalkyl groups include cyclopropyl, cyclobutyl, cyclopentyl, cyclohexyl, cycloheptyl, and cyclooctyl groups. The cycloalkyl group can also be a polycyclic (e.g., bicyclic) group by either possessing a bond between two ring groups (e.g., dicyclohexyl) or a shared (i.e., fused) side (e.g., decalin and norbornane).

In a fourth set of embodiments, the hydrocarbon group (R) is unsaturated and straight-chained, i.e., a straight-chained (linear) olefinic or alkenyl group. The unsaturation occurs by the presence of one or more carbon-carbon double bonds and/or one or more carbon-carbon triple bonds. Some examples of straight-chained olefinic groups include vinyl, propen-1-yl (allyl), 3-buten-1-yl (CH₂═CH—CH₂—CH₂—), 2-buten-1-yl (CH₂—CH═CH—CH₂—), butadienyl, 4-penten-1-yl, 3-penten-1-yl, 2-penten-1-yl, 2,4-pentadien-1-yl, 5-hexen-1-yl, 4-hexen-1-yl, 3-hexen-1-yl, 3,5-hexadien-1-yl, 1,3,5-hexatrien-1-yl, 6-hepten-1-yl, ethynyl, propargyl (2-propynyl), 3-butynyl, and the numerous other straight-chained alkenyl or alkynyl groups having up to 20 or 30 carbon atoms.

In a fifth set of embodiments, the hydrocarbon group (R) is unsaturated and branched, i.e., a branched olefinic or alkenyl group. Some examples of branched olefinic groups include propen-2-yl (CH₂═C·—CH₃), 1-buten-2-yl (CH₂═C·—CH₂—CH₃), 1-buten-3-yl (CH₂═CH—CH·—CH₃), 1-propen-2-methyl-3-yl (CH₂═C(CH₃)—CH₂—), 1-penten-4-yl, 1-penten-3-yl, 1-penten-2-yl, 2-penten-2-yl, 2-penten-3-yl, 2-penten-4-yl, and 1,4-pentadien-3-yl, and the numerous other branched alkenyl groups having up to 20 or 30 carbon atoms, wherein the dot in any of the foregoing groups indicates a point of attachment.

In a sixth set of embodiments, the hydrocarbon group (R) is unsaturated and cyclic, i.e., a cycloalkenyl group. The unsaturated cyclic group can be aromatic or aliphatic. Some examples of unsaturated cyclic hydrocarbon groups include cyclopropenyl, cyclobutenyl, cyclopentenyl, cyclopentadienyl, cyclohexenyl, cyclohexadienyl, phenyl, benzyl, cycloheptenyl, cycloheptadienyl, cyclooctenyl, cyclooctadienyl, and cyclooctatetraenyl groups. The unsaturated cyclic hydrocarbon group may or may not also be a polycyclic group (such as a bicyclic or tricyclic polyaromatic group) by either possessing a bond between two of the ring groups (e.g., biphenyl) or a shared (i.e., fused) side, as in naphthalene, anthracene, phenanthrene, phenalene, or indene fused ring systems. All of the foregoing cyclic groups are carbocyclic groups.

One or more of the hydrocarbon groups (R) may also include one or more heteroatoms (i.e., non-carbon and non-hydrogen atoms), such as one or more heteroatoms selected from oxygen, nitrogen, sulfur, silicon, phosphorus, boron, and halogen atoms, as well as groups containing one or more of these heteroatoms (i.e., heteroatom-containing groups). The heteroatom-containing groups may be present as substituents in any of the hydrocarbon groups described herein. Some examples of oxygen-containing groups include hydroxy (OH), alkoxy (OR′), carbonyl-containing (e.g., carboxylic acid, ketone, aldehyde, carboxylic ester, amide, and urea functionalities), nitro (NO₂), carbon-oxygen-carbon (ether), sulfonyl, and sulfinyl (i.e., sulfoxide) groups. Some particular examples of alkoxy groups —OR′ include methoxy, ethoxy, n-propoxy, isopropoxy, n-butoxy, isobutoxy, t-butoxy, phenoxy, benzyloxy, 2-hydroxyethoxy, 2-methoxyethoxy, 2-ethoxyethoxy, vinyloxy, and allyloxy groups. In the case of an ether group, the ether group can also be a polyalkyleneoxide (polyalkyleneglycol) group, such as a polyethyleneoxide group. Some examples of nitrogen-containing groups include primary amine, secondary amine, tertiary amine (i.e., —NR₂ or NR₃⁺, wherein R is independently selected from H and hydrocarbon groups set forth above), nitrile, amide (i.e., —C(O)NR₂ or —NRC(O)R, wherein R is independently selected from hydrogen atom and hydrocarbon groups set forth above), imine (e.g., —CR═NR, wherein R is independently H or a hydrocarbon group), oxime (—CR═N—OH), amidoxime (—C(NH₂)═N—OH), nitro, urea (—NR—C(O)—NR₂, wherein R is independently H or a hydrocarbon group), and carbamate groups (—NR—C(O)—OR, wherein R is independently H or a hydrocarbon group). Some examples of phosphorus-containing groups include —PR₂, —PR₃⁺, —P(═O)R₂, —P(OR)₂, —O—P(OR)₂, —R—P(OR)₂, —P(═O)(OR)₂, —O—P(═O)(OR)₂, —O—P(═O)(OR)(R), —O—P(═O)R₂, —R—P(═O)(OR)₂, —R—P(═O)(OR)(R), and —R—P(═O)R₂ groups, wherein R is independently selected from hydrogen atom and hydrocarbon groups set forth above. Some examples of sulfur-containing groups include mercapto (i.e., —SH), thioether (i.e., sulfide, e.g., —SR), disulfide (—R—S—S—R), sulfoxide (—S(O)R), sulfone (—SO₂R), sulfonate (—S(═O)₂OR, wherein R is H, a hydrocarbon group, or a cationic group), and sulfate groups (—OS(═O)₂OR, wherein R is H, a hydrocarbon group, or a cationic group). Some examples of halide atoms include fluorine, chlorine, bromine, and iodine. In some embodiments, one or more of any of the heteroatoms described above (e.g., oxygen, nitrogen, and/or sulfur atoms) or heteroatom groups are inserted between carbon atoms (e.g., as —O—, —NR—, or —S—) in any of the hydrocarbon groups described above to form a heteroatom-substituted hydrocarbon group. Alternatively, or in addition, one or more of the heteroatom-containing groups can replace one or more hydrogen atoms in the hydrocarbon group.

In some embodiments, the hydrocarbon group (R) is or includes a cyclic or polycyclic group that includes at least one ring heteroatom (for example, one, two, three, four, or higher number of heteroatoms). Such ring heteroatom-substituted cyclic groups are referred to herein as “heterocyclic groups.” As used herein, a “ring heteroatom” is an atom other than carbon and hydrogen (typically, selected from nitrogen, oxygen, and sulfur) that is inserted into, or replaces a ring carbon atom in, a hydrocarbon ring structure. In some embodiments, the heterocyclic group is saturated, while in other embodiments, the heterocyclic group is unsaturated. An unsaturated heterocyclic group may be aliphatic or aromatic, wherein an aromatic heterocyclic group is also referred to herein as a “heteroaromatic ring,” or a “heteroaromatic fused-ring system” in the case of at least two fused rings, at least one of which contains at least one ring heteroatom. In some embodiments, the heterocyclic group is bound via one of its ring carbon atoms to another group (i.e., other than hydrogen atom and adjacent ring atoms), while the one or more ring heteroatoms are not bound to another group. In other embodiments, the heterocyclic group is bound via one of its heteroatoms to another group, while ring carbon atoms may or may not be bound to another group.

Some examples of saturated heterocyclic groups (R) containing at least one oxygen atom include oxetane, tetrahydrofuran, tetrahydropyran, 1,4-dioxane, 1,3-dioxane, and 1,3-dioxepane rings. Some examples of saturated heterocyclic groups (R) containing at least one nitrogen atom include pyrrolidine, piperidine, piperazine, imidazolidine, azepane, and decahydroquinoline rings. Some examples of saturated heterocyclic groups (R) containing at least one sulfur atom include tetrahydrothiophene, tetrahydrothiopyran, 1,4-dithiane, 1,3-dithiane, and 1,3-dithiolane rings. Some examples of saturated heterocyclic groups (R) containing at least one oxygen atom and at least one nitrogen atom include morpholine and oxazolidine rings. An example of a saturated heterocyclic group containing at least one oxygen atom and at least one sulfur atom includes 1,4-thioxane. An example of a saturated heterocyclic group containing at least one nitrogen atom and at least one sulfur atom includes thiazolidine and thiamorpholine rings. Saturated heterocyclic linkers (R) can be derived from any of the foregoing saturated heterocyclic groups by removal of one, two, or three hydrogen atoms from the saturated heterocyclic group to result in a divalent, trivalent, or tetravalent saturated heterocyclic linker, respectively.

Some examples of unsaturated heterocyclic groups (R) containing at least one oxygen atom include furan, pyran, 1,4-dioxin, benzofuran, dibenzofuran, and dibenzodioxin rings. Some examples of unsaturated heterocyclic groups (R) containing at least one nitrogen atom include pyrrole, imidazole, pyrazole, pyridine, pyrazine, pyrimidine, 1,3,5-triazine, azepine, diazepine, indole, purine, benzimidazole, indazole, 2,2′-bipyridine, quinoline, isoquinoline, phenanthroline, 1,4,5,6-tetrahydropyrimidine, 1,2,3,6-tetrahydropyridine, 1,2,3,4-tetrahydroquinoline, quinoxaline, quinazoline, pyridazine, cinnoline, 5,6,7,8-tetrahydroquinoxaline, 1,8-naphthyridine, and 4-azabenzimidazole rings. Some examples of unsaturated heterocyclic groups containing at least one sulfur atom include thiophene, thianaphthene, and benzothiophene rings. Some examples of unsaturated heterocyclic groups (R) containing at least one oxygen atom and at least one nitrogen atom include oxazole, isoxazole, benzoxazole, benzisoxazole, oxazoline, 1,2,5-oxadiazole (furazan), and 1,3,4-oxadiazole rings. Some examples of unsaturated heterocyclic groups (R) containing at least one nitrogen atom and at least one sulfur atom include thiazole, isothiazole, benzothiazole, benzoisothiazole, thiazoline, and 1,3,4-thiadiazole rings. Unsaturated heterocyclic linkers (R) can be derived from any of the foregoing unsaturated heterocyclic groups by removal of one, two, or three hydrogen atoms from the unsaturated heterocyclic group to result in a divalent, trivalent, or tetravalent unsaturated heterocyclic linker, respectively.

In a first aspect, the present disclosure is directed to covalent inhibitor molecules of coronavirus papain-like protease (PLpro) having the following structure:

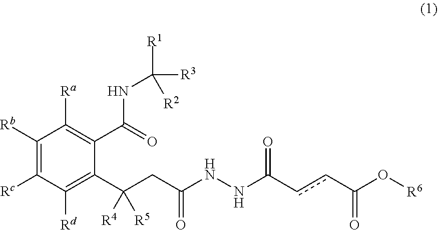

or more particularly,

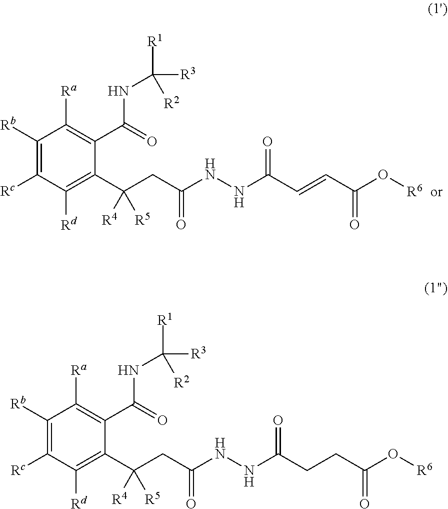

The variable R¹ in Formula (1) is a hydrocarbon group containing at least one aromatic or heteroaromatic ring or fused ring system, as described for such R groups earlier above. R¹ may be or contain (include), for example, a phenyl, naphthyl, quinoline, isoquinoline, pyridyl, pyrrolyl, imidazolyl, pyrazinyl, pyrazolyl, indolyl, oxazolyl, purinyl, quinazolinyl, triazinyl, pyrimidinyl, triazolyl, thiazolyl, thienyl, furyl, cyclopentyl, cyclohexyl, cyclopentadienyl, or any two of the foregoing rings connected (bound) to each other, either directly or through a linker (e.g., methylene, ethylene, carbonyl, ether, amino, amide, or combination thereof), or any two of the foregoing rings fused to each other. Any of the foregoing rings or fused ring systems may or may not contain one or more substituents, as described earlier above. More particularly, any of the foregoing rings are optionally substituted with one or more groups selected from halogen atoms, hydrocarbon groups containing 1-6 carbon atoms; OR′ groups; C(O)OR′ groups; amine groups of the formula NR′₂; and amide groups of the formula NHC(O)R′ or C(O)NR′₂, wherein R′ is independently selected from H and hydrocarbon groups containing 1-6 carbon atoms. In some embodiments, R¹ is not naphthyl or does not include a naphthyl ring. In other embodiments, R¹ is not quinolinyl or isoquinolinyl or does not include a quinolinyl or isoquinolinyl ring.

In specific embodiments, R¹ may be selected from any of the foregoing ring-containing groups:

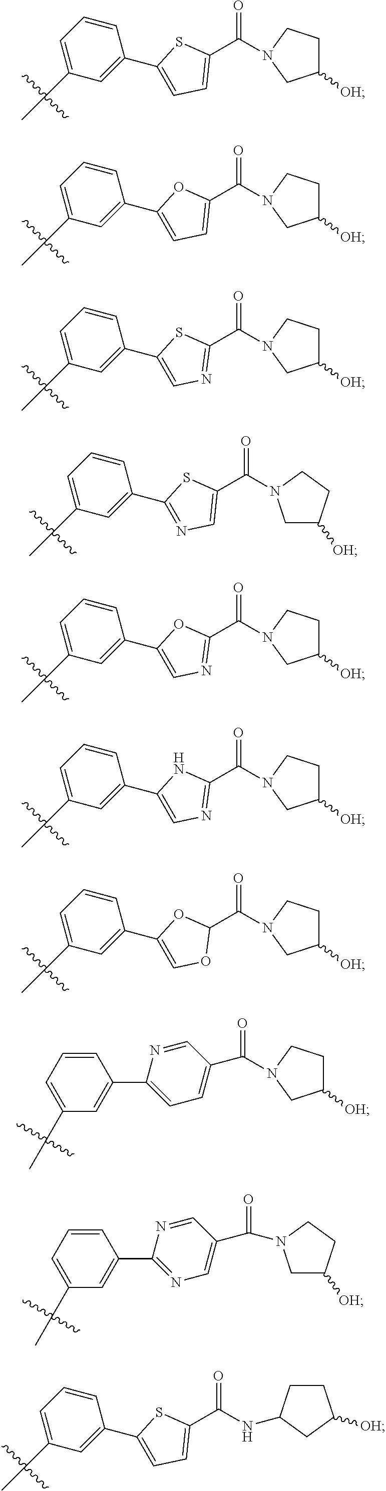

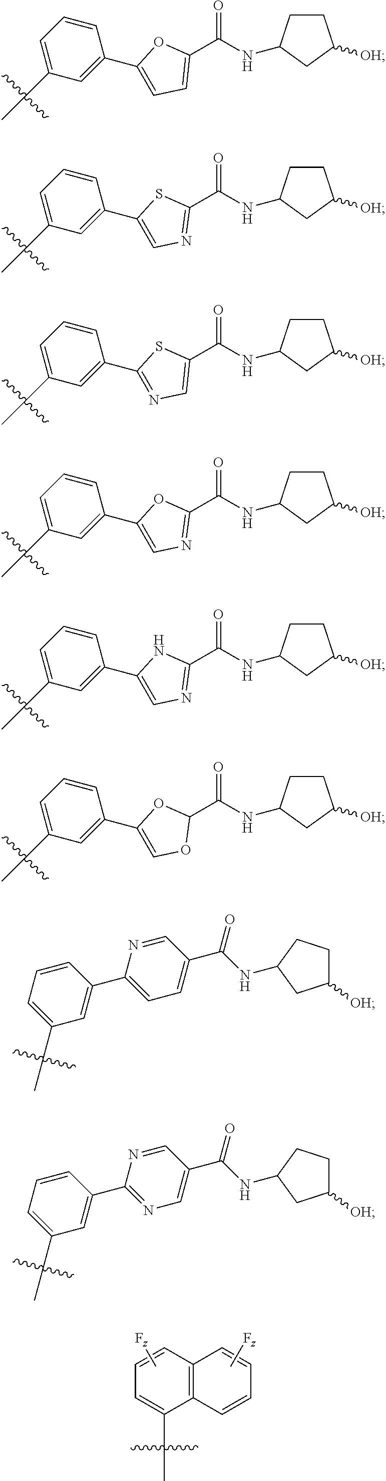

(i.e., fluorinated naphthyl, wherein z is independently 0, 1, 2, 3, or 4 in each instance, which respectively correspond to 0, 1, 2, 3, or 4 fluorine (F) atoms per ring, provided that the sum of both z variables is greater than 0); and

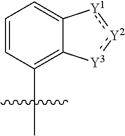

wherein Y¹, Y², and Y³ are independently selected from N, NH, N(CH₃), S, O, C, or CH, and the dashed lines represent optional double bonds. In some embodiments, any one or two of the carbon atoms in the shown benzene ring (i.e., those carbon atoms that are not fused with the other shown ring) may be substituted with an N atom. In the above structures, the wavy lines shown crossing a bond represent a connection point of the structure, whereas the wavy lines connected to OH represent a bond oriented above or below the ring, which results in two different enantiomers. In the above structures, the dashed carbonyl group indicates the presence or absence of a carbonyl group. If the carbonyl group is absent, it is replaced with a methylene (CH₂) group (i.e., the shown oxygen atom of the carbonyl group is replaced with two hydrogen atoms attached to the carbon atom of the carbonyl group).

The variables R² and R³ in Formula (1) are independently selected from hydrogen atom (H), halogen atom (e.g., F, Cl, or Br), and hydrocarbon groups containing 1-6 carbon atoms optionally substituted with one or more halogen atoms and optionally containing an —O— or —NH— linkage. The hydrocarbon groups containing 1-6 carbon atoms (or more specifically, 1-5, 1-4, 1-3, 2-6, 3-6, or 4-6 carbon atoms) may be any such hydrocarbon groups (e.g., alkyl, alkenyl, cycloalkyl, aromatic, or heteroaromatic), and may or may not contain one or more heteroatoms, as amply described earlier above. In some embodiments, at least one of R² and R³ is H, or both of R² and R³ are H. In other embodiments, at least one of R² and R³ is a hydrocarbon containing 1-6 carbon atoms, or both of R² and R³ are independently hydrocarbon groups containing 1-6 carbon atoms. In other embodiments, at least one of R² and R³ is a halogen atom, or both of R² and R³ are independently halogen atoms. In other embodiments, R³ is H and R² is a hydrocarbon group containing 1-6 carbon atoms optionally substituted with one or more F atoms (e.g., CH₃ or CF₃). In other embodiments, R³ is H and R² is a halogen atom. In some embodiments, R² and R³ are interconnected to form a three-, four-, five-, or six-membered cycloalkyl or heterocycloalkyl ring. In the case of a heterocycloalkyl ring, this may contain any of the heteroatoms described earlier above within the ring, or more typically, one or more of O, N, and/or S, and the heterocycloalkyl ring is typically connected to the shown position in Formula (1) by a carbon atom. The interconnected ring may be, for example, a cyclopropyl, cyclobutyl, cyclopentyl, cyclohexyl, oxirane, oxetane, tetrahydrofuran, 1,3-dioxolane, aziridine, azetidine, pyrrolidine, imidazolidine, oxazolidine, or piperidine ring.

The variables R⁴ and R⁵ in Formula (1) are independently selected from H (or more specifically D), halogen atom, and hydrocarbon groups containing 1-6 carbon atoms optionally substituted with one or more halogen atoms and optionally containing an —O— or —NH— linkage. The hydrocarbon groups containing 1-6 carbon atoms (or more specifically, 1-5, 1-4, 1-3, 2-6, 3-6, or 4-6 carbon atoms) may be any such hydrocarbon groups (e.g., alkyl, alkenyl, cycloalkyl, aromatic, or heteroaromatic), and may or may not contain one or more heteroatoms, as amply described earlier above. In some embodiments, at least one of R⁴ and R⁵ is H, or both of R⁴ and R⁵ are H. In other embodiments, at least one of R⁴ and R⁵ is a hydrocarbon containing 1-6 carbon atoms, or both of R⁴ and R⁵ are independently hydrocarbon groups containing 1-6 carbon atoms. In other embodiments, at least one of R⁴ and R⁵ is a halogen atom, or both of R⁴ and R⁵ are independently halogen atoms (or more specifically, one or both of R⁴ and R⁵ are fluorine atoms). In other embodiments, R⁴ is H and R⁵ is a hydrocarbon group containing 1-6 carbon atoms. In other embodiments, R⁴ is H and R⁵ is a halogen atom. In some embodiments, at least one of R⁴ and R⁵ is a halogen atom or a hydrocarbon group containing 1-6 carbon atoms optionally substituted with one or more halogen atoms and optionally containing an —O— or —NH— linkage, wherein R⁴ and R⁵ are optionally interconnected as above. In some embodiments, R⁴ and R⁵ are interconnected to form a three-, four-, five-, or six-membered cycloalkyl or heterocycloalkyl ring. In the case of a heterocycloalkyl ring, this may contain any of the heteroatoms described earlier above within the ring, or more typically, one or more of O, N, and/or S, and the heterocycloalkyl ring is typically connected to the shown position in Formula (1) by a carbon atom. The interconnected ring may be, for example, a cyclopropyl, cyclobutyl, cyclopentyl, cyclohexyl, oxirane, oxetane, tetrahydrofuran, 1,3-dioxolane, aziridine, azetidine, pyrrolidine, imidazolidine, oxazolidine, or piperidine ring.

The variable R⁶ in Formula (1) is H or a hydrocarbon group containing 1-12 carbon atoms and optionally containing one or more heteroatoms selected from O, N, S, and F. In some embodiments, R⁶ is a hydrocarbon group containing 1-12 carbon atoms, optionally substituted with one or more F atoms. In a first set of embodiments, R⁶ is a linear, branched, or cyclic alkyl, alkenyl, or alkynyl hydrocarbon group containing 1, 2, 3, 4, 5, or 6 carbon atoms (e.g., 1-6, 1-4, 1-3, or 3-6 carbon atoms), such as any of those described earlier above (e.g., methyl, fluoromethyl, difluoromethyl, trifluoromethyl, ethyl, 2,2,2-trifluoroethyl, perfluoroethyl, n-propyl, isopropyl, cyclopropyl, n-butyl, isobutyl, sec-butyl, t-butyl, cyclobutyl, n-pentyl, isopentyl, neopentyl, cyclopentyl, n-hexyl, isohexyl, 3,3-dimethylbutyl, cyclohexyl, and phenyl). In some embodiments, R⁶ is a hydrocarbon group containing 7, 8, 9, 10, 11, or 12 carbon atoms, such as any of those described earlier above, including aromatic rings (e.g., n-heptyl, isoheptyl, n-octyl, isooctyl, benzyl, tolyl, xylyl, mesityl, naphthyl, diphenyl, and norbornyl). In a second set of embodiments, R⁶ has the formula —(CH₂)ᵣ—U, wherein U is a carbocyclic or heterocyclic group and r is an integer of 0, 1, 2, or 3 (e.g., 0-3 or 1-3), and wherein U may be attached to any carbon atom of the linker —(CH₂)ᵣ—, such as the terminal carbon. The group U can be selected from any of the carbocyclic or heterocyclic groups described earlier above, including aromatic and heteroaromatic groups described earlier above. In a third set of embodiments, R⁶ is a hydrocarbon group containing 1-12 carbon atoms and at least one alkyl halide group, wherein the halide may be F, Cl, Br, or I. In some embodiments, R⁶ is an alkyl halide group having the formula —(CH₂)ₛ—W, wherein W is halogen (F, Cl, Br, or I) and subscript s is 0, 1, 2, or 3 (e.g., 0-3, 1-2, or 1-3) and wherein W may be attached to any carbon atom of the linker —(CH₂)ₛ—, such as the terminal carbon. In a fourth set of embodiments, R⁶ is a hydrocarbon group containing 1-12 carbon atoms and at least one nitrile group. In some embodiments, R⁶ is an alkyl nitrile group having the formula —(CH₂)ₜ—CN, wherein subscript t is 0, 1, 2, or 3 (e.g., 0-3, 1-2, or 1-3) and wherein CN may be attached to any carbon atom of the linker —(CH₂)ₜ—, such as the terminal carbon. In some embodiments, R⁶ is selected from —CH₃, —CF₃, —CH₂—CH₃, or —CH₂—Y, wherein Y is an aromatic ring, e.g., C₆H₅ or 5- and 6-membered heterocycles containing one or more ring heteroatoms selected from N, O, and/or S.

In specific embodiments, R⁶ may be selected from any of the foregoing ring-containing groups:

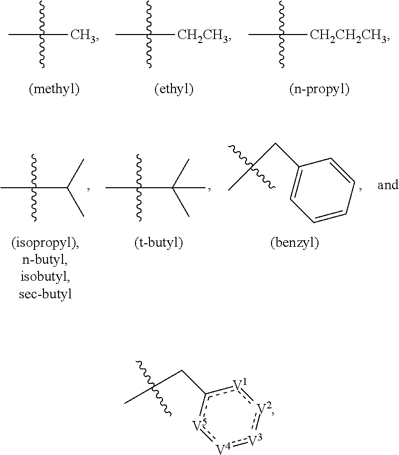

wherein V¹-V⁵ independently represent N, NH, N(CH₃), N(CH₂CH₃), S, O, C, or CH, and the dashed lines represent optional double bonds. In the above structures, the wavy lines represent a connection point of the structure.

The variables R^{a}, R^{b}, R^{c}, and R^{d} are independently selected from H; halogen atoms (e.g., F, Cl, Br); hydrocarbon groups containing 1-3 carbon atoms (e.g., methyl, ethyl, n-propyl, isopropyl, cyclopropyl, or allyl, and fluorinated versions thereof); amine groups of the formula NR′₂; amide groups of the formula NHC(O)R′ or C(O)NHR′; carbamate groups of the formula NHC(O)OR′; urea groups of the formula NHC(O)NR′₂; and —(CH₂)ₚ-T, wherein T is a hydrocarbon group containing at least one —NH— linkage. In the foregoing groups, R′ is independently selected from H and hydrocarbon groups containing 1-6 (or 1-4, 1-3, 2-6, 3-6, or 4-6) carbon atoms, and p is an integer of 0-3. In one set of embodiments, one, two, or more (or all) of R^{a}, R^{b}, R^{c}, and R^{d} is/are H. In another set of embodiments, one, two, or more of R^{a}, R^{b}, R^{c}, and R^{d} is/are selected from halogen atoms, typically fluorine and/or chlorine atoms. In another set of embodiments, one, two, or more of R^{a}, R^{b}, R^{c}, and R^{d} is/are selected from hydrocarbon groups containing 1-3 carbon atoms. In specific embodiments, the hydrocarbon groups containing 1-3 carbon atoms are selected from, for example, methyl, ethyl, n-propyl, isopropyl, cyclopropyl, and fluorinated versions thereof (e.g., fluoromethyl, difluoromethyl, trifluoromethyl, 2-fluoroethyl, and 2,2,2-trifluoroethyl groups). In another set of embodiments, one, two, or more of R^{a}, R^{b}, R^{c}, and R^{d} is/are selected from amine groups of the formula NR′₂. In another set of embodiments, one, two, or more of R^{a}, R^{b}, R^{c}, and R^{d} is/are selected from amide groups of the formula NHC(O)R′. In another set of embodiments, one, two, or more of R^{a}, R^{b}, R^{c}, and R^{d} is/are selected from carbamate groups of the formula NHC(O)OR or urea groups of the formula NHC(O)NR′₂. In another set of embodiments, one, two, or more of R^{a}, R^{b}, R^{c}, and R^{d} is/are selected from groups of the formula —(CH₂)ₚ-T, wherein T is a hydrocarbon group containing at least one —NH— linkage, and p is an integer of 0-3. Notably, any two or more of the foregoing embodiments may be combined. In some embodiments, R^{a} is selected from halogen atoms (e.g., F, Cl, Br); hydrocarbon groups containing 1-3 carbon atoms (e.g., methyl, ethyl, n-propyl, isopropyl, cyclopropyl, allyl, and fluorinated versions thereof); amine groups of the formula NR′₂; amide groups of the formula NHC(O)R′; and —(CH₂)ₚ-T, wherein T is a hydrocarbon group containing at least one —NH— linkage, while R^{b}, R^{c}, and R^{d} is/are selected from H and any one or more of the foregoing groups, or R^{b}, R¹, and R^{d} are all H.

In some embodiments, one, two, or more of R^{a}, R^{b}, R^{c}, and R^{d} is/are selected from NR′₂, wherein R′ is independently selected from H and hydrocarbon groups containing 1-6 carbon atoms. In some embodiments, both R′ are H atoms, which corresponds to R^{a}, R^{b}, R^{c}, and/or R^{d} being NH₂. In other embodiments, one R′ is H and the other is a hydrocarbon group containing 1-6 carbon atoms. In other embodiments, both R′ are hydrocarbon groups containing 1-6 carbon atoms, wherein the hydrocarbon groups may be the same or different. R′ may be independently selected from any of the hydrocarbon groups described above containing 1-6 carbon atoms. In particular embodiments, one or both R′ are selected from linear or branched alkyl groups containing 1-6 carbon atoms, such as methyl, ethyl, n-propyl, n-butyl, n-pentyl, n-hexyl, isopropyl, isobutyl, sec-butyl, t-butyl, isopentyl, neopentyl, and isohexyl groups.

In other embodiments, one, two, or more of R^{a}, R^{b}, R^{c}, and R^{d} is/are selected from NHC(O)R′, wherein R′ is selected from H and hydrocarbon groups containing 1-6 carbon atoms. In one embodiment, R′ is H, which corresponds to R^{a}, R^{b}, R^{c}, and/or R^{d} being NHC(O)H. In other embodiments, R′ is a hydrocarbon group (or alkyl group) containing 1-6 carbon atoms, wherein the hydrocarbon groups may be the same or different. R′ may be selected from any of the hydrocarbon groups described above containing 1-6 carbon atoms. In particular embodiments, R′ is selected from linear or branched alkyl groups containing 1-6 carbon atoms, such as methyl, ethyl, n-propyl, n-butyl, n-pentyl, n-hexyl, isopropyl, isobutyl, sec-butyl, t-butyl, isopentyl, neopentyl, and isohexyl groups.

In other embodiments, one, two, or more of R^{a}, R^{b}, R^{c}, and R^{d} is/are selected from —(CH₂)ₚ-T, wherein T contains at least one —NH— linkage, and p is an integer of 0-3. In some embodiments, T contains at least one —NH— linkage and a carbonyl (—CO—) linkage, which may be separate or may be combined to form an amide or urea linkage. T typically contains 1, 2, or 3 carbon atoms. In some embodiments, T may be or include a guanidine or guanidinium group. In alternative embodiments, T may include at least one —NH— linkage and a saturated or unsaturated N-containing heterocycle, such as any of those described earlier above.

In yet other embodiments, one or more of R^{a}, R^{b}, R^{c}, and R^{d} (or more specifically, R^{a}) represents a group substituted in the benzamide region of (R)—N-(1-(naphthalen-1-yl)ethyl)benzamide core. The foregoing group is intended to generate favorable interactions with solvent water and surrounding amino acids, and in some embodiments, may generate a positively charged group. In more specific embodiments, one or more of R^{a}, R^{b}, R^{c}, and R^{d} (or more specifically, R^{a}) is selected from —H, —NH₂, —NHAc, (CH₂)_{1,2,3}—NH—C(NH)(NH₂), (CH₂)_{1,2,3}—C(O)—NH—C(NH)(NH₂), —(CH₂)_{1,2,3}—NH—X, where X═N— containing heterocycles and substituted N-containing heterocycles that mimic arginine amino acids by generating positively charged groups (e.g., as shown in FIG. 1) that are PLpro inhibitors and are useful for treating SARS-CoV-2 or related coronavirus infections and disease symptoms associated with such viruses.

In specific embodiments of Formula (1), the compound has the following structure:

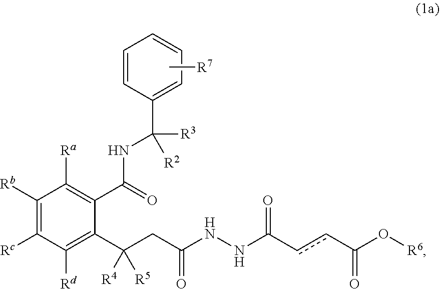

or more particularly,

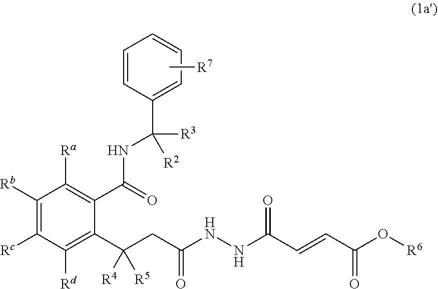

In Formula (1a), the variables R², R³, R⁴, R⁵, R⁶, R^{a}, R^{b}, R^{c}, and R^{d} are as defined under Formula (1) above, including all of the possible embodiments and combinations thereof described above under Formula (1). In some embodiments of Formula (1a), one or both of R⁴ and R⁵ are fluorine atoms. In further or separate embodiments of Formula (1a), R² and R³ are interconnected to form a three-, four-, five-, or six-membered cycloalkyl or heterocycloalkyl ring, as described above under Formula (1).

The variable R⁷ in Formula (1a) is or includes a heterocyclic ring or ring system, any of which may be a substituent of or fused to the shown phenyl ring to which R⁷ is attached. The heterocyclic ring or ring system may be any of those described earlier above, and may contain one or more ring atoms selected from N, O, and/or S and may be saturated, aliphatic, or aromatic. More typically, R⁷ is a five-membered or six-membered heterocyclic ring, such as any of those described earlier above, or R⁷ is more specifically a five-membered or six-membered heteroaromatic ring or fused ring system, such as any of those described earlier above. In more specific embodiments, R⁷ may be or include a thiazole, pyrrolidine, pyrrole, 2,3-dihydro-1H-pyrrole, furan, thiophene, imidazole, oxazole, isothiazole, 1,3-dioxolane, 1,3-dioxole, pyridine, pyrazine, piperidine, piperazine, pyrimidine, pyran, 1,3-dioxane, or 1,3-dioxine ring, or R⁷ may be or include a ring system in which any of the foregoing rings are bound or fused to each other either directly or through a linker (e.g., thiazole ring bound to a pyrrolidine ring optionally through a linker, such as —(CH₂)_{w}— where w is 0, 1, 2, or 3), and/or R⁷ may be or include a ring system in which any of the foregoing rings are bound or fused to a benzene ring (e.g., indole ring). In specific embodiments, R⁷ may be or include a thiazole, thiophene, and/or pyrrolidine ring. Any of the rings or ring systems in R⁷ may or may not also be substituted with one or more substituents, such as any of the hydrocarbon groups (R), halogen atoms, or heteroatom-containing groups described above. In some embodiments, one or more rings present in R⁷ may be substituted with one or more OH, alkoxy (OR), amine, or amide groups or one or more halogen atoms (particularly F and/or Cl).

Any of the compounds described herein according to Formula (1) and sub-formulas thereof may be enantiomerically enriched, enantiomerically pure (isolated), or racemic mixture (racemate). For the sake of simplicity, the compounds of the present invention have been thus far depicted without an indication of chirality. However, Formula (1) and sub-formulas thereof are intended to include all possible enantiomers and racemic mixtures. A first stereocenter may occur at the carbon attached to R² and R³ if R² and R³ are different. If R² and R³ are interconnected, a stereocenter may still occur if the interconnected ring is asymmetrical. A stereocenter may occur at the carbon attached to R² and R³ if R² and R³ are different. An enantiomer will result if a first stereocenter is present in the absence of a second stereocenter or if a second stereocenter is present in the absence of a first stereocenter. A diastereomer will result if first and second stereocenters are both present. Diastereomerically enriched, diastereomerically pure (isolated), or diastereomeric mixtures of any of the compounds described herein are also considered herein. A stereocenter may alternatively or in addition be present within a variable group (e.g., within a group R¹).

For example, compounds of Formula (1) include the following two enantiomers, wherein R³ is H and thus not shown:

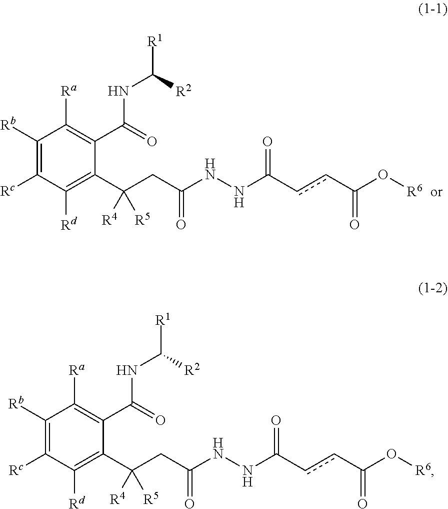

or more particularly,

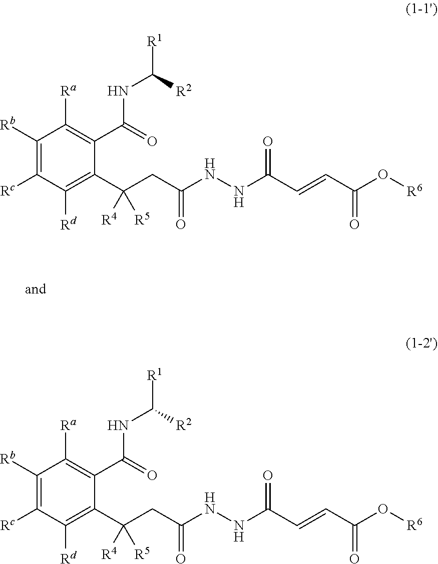

In Formulas (1-1) and (1-2), R² is a halogen atom or a hydrocarbon group containing 1-6 carbon atoms optionally substituted with one or more halogen atoms and optionally containing an —O— or —NH— linkage; and R¹, R⁴, R⁵, R⁶, R^{a}, R^{b}, R^{c}, and R^{d} are as defined under Formula (1) including all of the possible embodiments and combinations thereof described above. In some embodiments of Formula (1-1) or (1-2), R¹ is not naphthyl or does not include a naphthyl ring. In other embodiments of Formula (1-1) or (1-2), R¹ is not quinolinyl or isoquinolinyl or does not include a quinolinyl or isoquinolinyl ring. In some embodiments of Formula (1-1) or (1-2), one or both of R⁴ and R⁵ are fluorine atoms. In further or separate embodiments of Formula (1-1) or (1-2), R² and R³ are interconnected to form a three-, four-, five-, or six-membered cycloalkyl or heterocycloalkyl ring, as described above under Formula (1). Enantiomerically enriched, enantiomerically pure, and racemic mixture (racemate) forms of any of the above enantiomers of Formula (1) are considered herein.

Similarly, compounds of Formula (1a) include the following two enantiomers, wherein R³ is H and thus not shown:

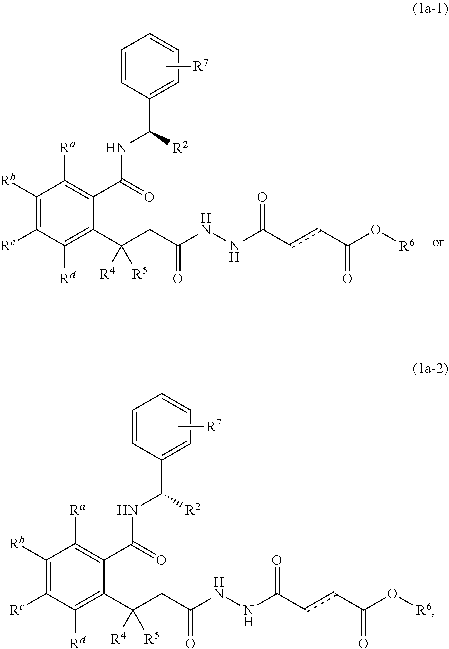

or more particularly,

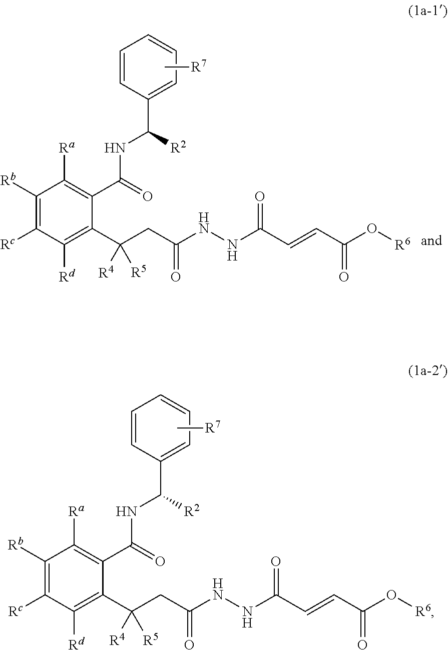

In Formulas (1a-1) and (1a-2), R² is a halogen atom or a hydrocarbon group containing 1-6 carbon atoms optionally substituted with one or more halogen atoms and optionally containing an —O— or —NH— linkage; and R¹, R⁴, R⁵, R⁶, R⁷, R^{a}, R^{b}, R^{c}, and R^{d} are as defined under Formulas (1) and (1a) including all of the possible embodiments and combinations thereof described above. In some embodiments of Formula (1a-1) or (1a-2), R¹ is not naphthyl or does not include a naphthyl ring. In other embodiments of Formula (1a-1) or (1a-2), R¹ is not quinolinyl or isoquinolinyl or does not include a quinolinyl or isoquinolinyl ring. In some embodiments of Formula (1a-1) or (1a-2), one or both of R⁴ and R⁵ are fluorine atoms. In further or separate embodiments of Formula (1a-1) or (1a-2), R² and R³ are interconnected to form a three-, four-, five-, or six-membered cycloalkyl or heterocycloalkyl ring, as described above under Formula (1). Enantiomerically enriched, enantiomerically pure, and racemic mixture (racemate) forms of any of the above enantiomers of Formula (1a) are considered herein.

In other embodiments of Formula (1), the compound has the following structure:

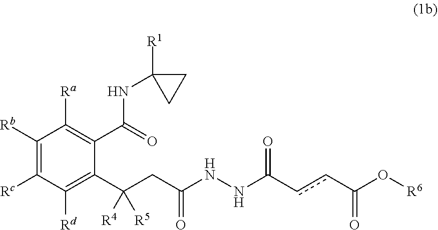

or more particularly,

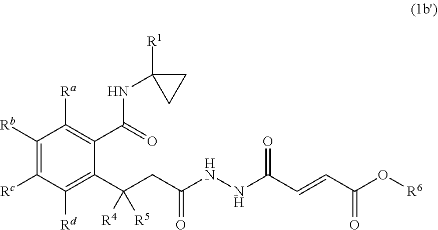

In Formula (1b), the variables R¹, R⁴, R⁵, R⁶, R^{a}, R^{b}, R^{c}, and R^{d} are as defined under Formula (1) above, including all of the possible embodiments and combinations thereof described above under Formula (1). In some embodiments of Formula (1b), one or both of R⁴ and R⁵ are fluorine atoms.

In other embodiments of Formula (1), the compound has the following structure:

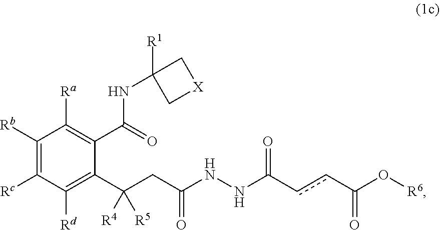

or more particularly,

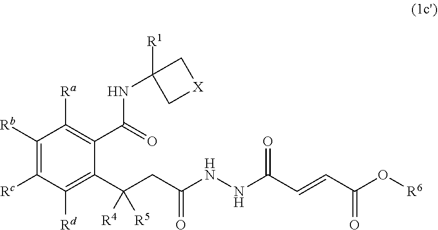

In Formula (1c), the variables R¹, R⁴, R⁵, R⁶, R^{a}, R^{b}, R^{c}, and R^{d} are as defined under Formula (1) above, including all of the possible embodiments and combinations thereof described above under Formula (1). In some embodiments of Formula (1b), one or both of R⁴ and R⁵ are fluorine atoms. The variable X is selected from CR^{e}R^{f}, O, S, and NR^{g}, wherein R^{e}, R^{f}, and R^{g} are independently selected from H and hydrocarbon groups containing 1-3 carbon atoms.

In some embodiments of Formula (1b) or (1c), R¹ is or includes naphthyl, quinolinyl, or isoquinolinyl, or conversely, R¹ may exclude any of the foregoing rings. In other embodiments, R¹ is or includes a thiazole, pyrrolidine, pyrrole, 2,3-dihydro-1H-pyrrole, furan, thiophene, imidazole, oxazole, isothiazole, 1,3-dioxolane, 1,3-dioxole, pyridine, pyrazine, piperidine, piperazine, pyrimidine, pyran, 1,3-dioxane, or 1,3-dioxine ring, or R¹ may be or include a ring system in which any of the foregoing rings are bound or fused to each other either directly or through a linker (e.g., thiazole ring bound to a pyrrolidine ring optionally through a linker, such as —(CH₂)_{w}— where w is 0, 1, 2, or 3), and/or R¹ may be or include a ring system in which any of the foregoing rings are bound or fused to a benzene ring (e.g., indole ring). In some embodiments, R¹ may be or include a thiazole, thiophene, and/or pyrrolidine ring.

In other embodiments of Formula (1), the compound has the following structure:

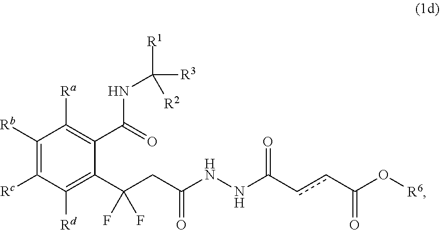

or more particularly,

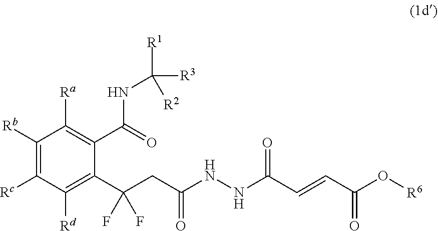

In Formula (1d), the variables R¹, R², R³, R⁶, R^{a}, R^{b}, R^{c}, and R^{d} are as defined under Formula (1) above, including all of the possible embodiments and combinations thereof described above under Formula (1). In some embodiments of Formula (1d), R¹ is or includes naphthyl, quinolinyl, or isoquinolinyl, or conversely, R¹ may exclude any of the foregoing rings. In other embodiments, R¹ is or includes a thiazole, pyrrolidine, pyrrole, 2,3-dihydro-1H-pyrrole, furan, thiophene, imidazole, oxazole, isothiazole, 1,3-dioxolane, 1,3-dioxole, pyridine, pyrazine, piperidine, piperazine, pyrimidine, pyran, 1,3-dioxane, or 1,3-dioxine ring, or R¹ may be or include a ring system in which any of the foregoing rings are bound or fused to each other either directly or through a linker (e.g., thiazole ring bound to a pyrrolidine ring optionally through a linker, such as —(CH₂)_{w}— where w is 0, 1, 2, or 3), and/or R¹ may be or include a ring system in which any of the foregoing rings are bound or fused to a benzene ring (e.g., indole ring). In some embodiments, R¹ may be or include a thiazole, thiophene, and/or pyrrolidine ring.

Some specific exemplary compounds within the scope of Formula (1) include the following:

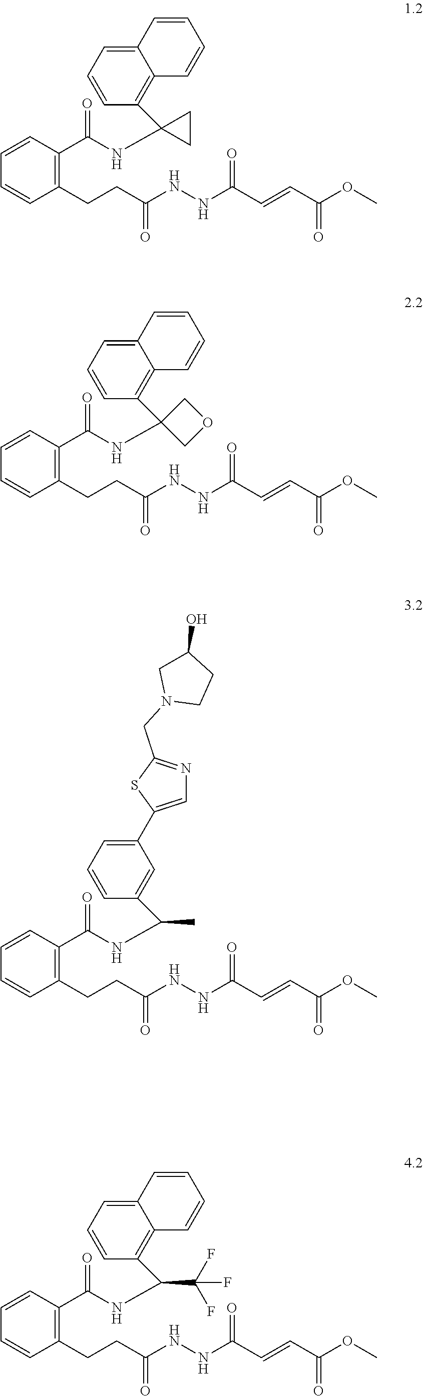 [SMILES: COC(=O)/C=C/C(=O)NNC(=O)CCc1ccccc1C(=O)NC1(c2cccc3ccccc23)CC1 ; COC(=O)/C=C/C(=O)NNC(=O)CCc1ccccc1C(=O)NC1(c2cccc3ccccc23)COC1 ; COC(=O)/C=C/C(=O)NNC(=O)CCc1ccccc1C(=O)N[C@H](C)c1cccc(-c2cnc(CN3CC[C@H](O)C3)s2)c1 ; COC(=O)/C=C/C(=O)NNC(=O)CCc1ccccc1C(=O)N[C@@H](c1cccc2ccccc12)C(F)(F)F]

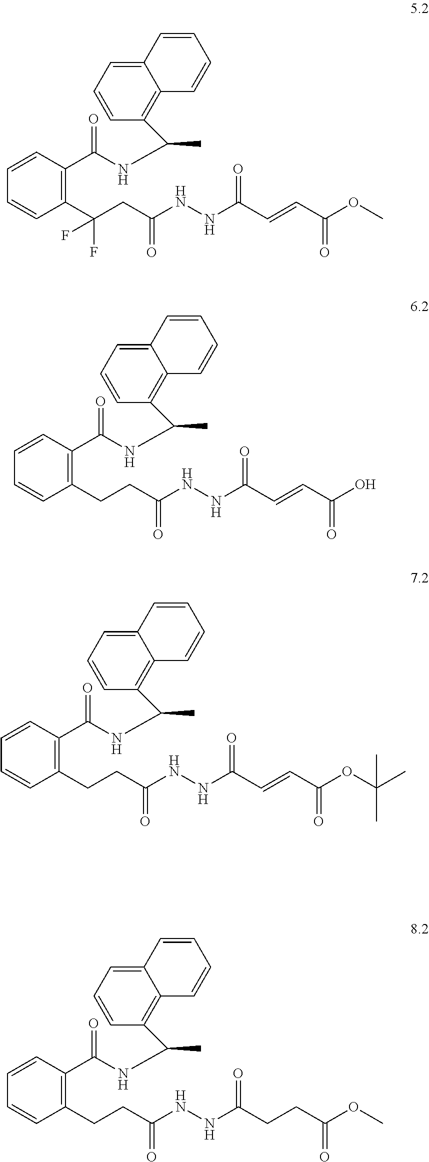 [SMILES: COC(=O)/C=C/C(=O)NNC(=O)CC(F)(F)c1ccccc1C(=O)N[C@H](C)c1cccc2ccccc12 ; C[C@@H](NC(=O)c1ccccc1CCC(=O)NNC(=O)/C=C/C(=O)O)c1cccc2ccccc12 ; C[C@@H](NC(=O)c1ccccc1CCC(=O)NNC(=O)/C=C/C(=O)OC(C)(C)C)c1cccc2ccccc12 ; COC(=O)CCC(=O)NNC(=O)CCc1ccccc1C(=O)N[C@H](C)c1cccc2ccccc12]

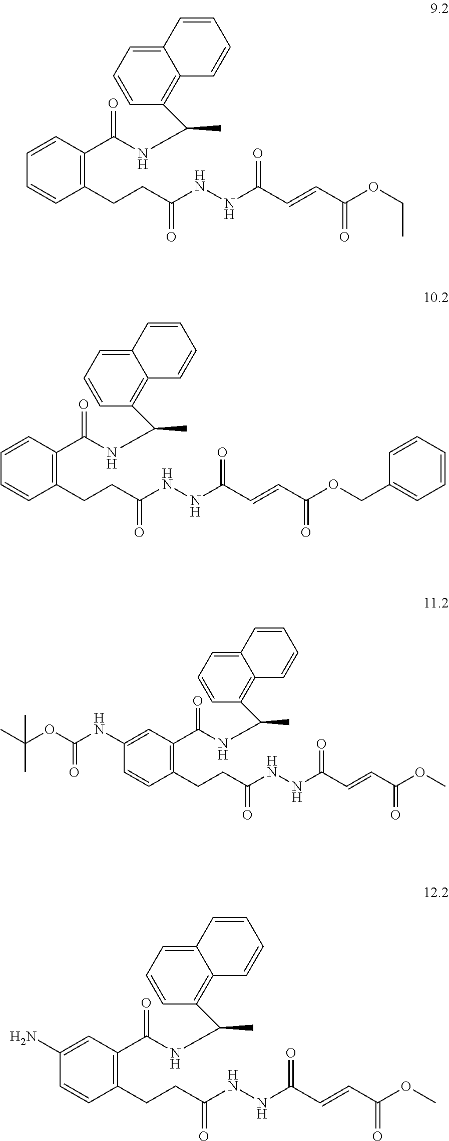

The compounds of Formula (1) and sub-formulas thereof can be synthesized by methods well known in the art, such as for example, Sanders et al. Nat. Commun. 2023 DOI: 10.1038/s41467-023-37254-w. A possible synthetic methodology is shown in the following scheme:

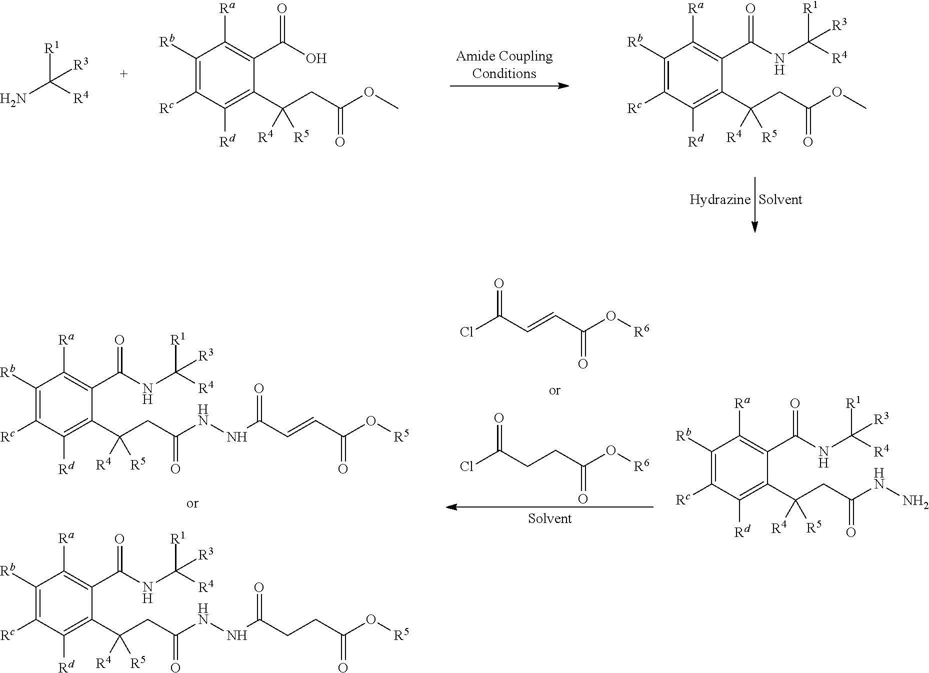

The present invention also relates to the combination of an inhibitor molecule of Formula (1) and any sub-formulas thereof together with one or more antimicrobial compounds, particularly antiviral compounds, and particularly those active against SARS-CoV-2. The antiviral compound having activity against SARS-CoV-2 typically targets SARS-CoV-2 proteins, such as the viral RNA-dependent RNA polymerase, or the 3CL protease. Some examples of such antivirals include, but are not limited to, remdesivir, molnupiravir, and nirmatrelvir.

Any of the compounds (i.e., “molecules”) of Formula (1) and sub-formulas thereof can function as coronavirus PLpro inhibitors, and are thus useful in treating or preventing a disease, disorder, or condition mediated by a coronavirus papain-like protease in its roles of generating the viral replicase complex and/or cleaving regulators of host immune pathways such as ubiquitin and ISG15. In particular embodiments, molecules of Formula (1) and sub-formulas thereof are useful in inhibiting PLpro activity, viral replicase complex formation, viral replication, and/or cleavage of host immune pathway regulators by coronaviruses such as SARS-CoV, SARS-CoV-2, and related viruses. The molecules of the present invention are also useful in the preparation and execution of screening assays for antiviral compounds. For example, the molecules can be useful for identifying PLpro enzyme mutants that are resistant to these molecules, such that they can be used as screening tools for more potent antiviral compounds. The molecules of the present invention can also be used to establish or determine the binding site of other antivirals to PLpro protease by, for example, competitive inhibition.

Any of the molecules according to Formula (1) and sub-formulas thereof may be in the form of a pharmaceutically acceptable salt. As used herein, the term “pharmaceutically acceptable salt” includes both acid and base addition salts, wherein the compound is modified by making acid or base salts thereof. As the molecules described herein typically include at least one amino group or linker (e.g., —NH—), acid addition salts are particularly considered. Examples of pharmaceutically acceptable salts include but are not limited to mineral or organic acid salts of basic residues such as amines. Pharmaceutically acceptable salts include conventional non-toxic salts or quaternary ammonium salts of the parent compound formed, for example, from non-toxic inorganic or organic acids. Such conventional non-toxic salts include those derived from inorganic acids such as hydrochloric, hydrobromic, sulfuric, sulfamic, phosphoric, and nitric acids; and the salts prepared from organic acids, such as acetic, propionic, succinic, glycolic, stearic, lactic, maleic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, naphthalene sulfonic, methanesulfonic, ethane disulfonic, and oxalic acids. The pharmaceutically acceptable salts of a compound disclosed herein can be synthesized from the parent compound, which contains a basic or acidic moiety, by conventional chemical methods. Suitable salts include those described in P. Heinrich Stahl, Camille G. Wermuth (Eds.), Handbook of Pharmaceutical Salts Properties, Selection, and Use, 2002.

In another aspect, the invention is directed to pharmaceutical compositions that contain any of the above-described compounds of Formula (1) and sub-formulas thereof dispersed in a pharmaceutically acceptable carrier, i.e., vehicle or excipient. The compound is dispersed in the pharmaceutically acceptable carrier by either being mixed (e.g., in solid form with a solid carrier) or dissolved or emulsified in a liquid carrier. The pharmaceutical composition may or may not also be formulated together with one or more additional active ingredients or adjuvants that improve the overall efficacy of the pharmaceutical composition, particularly as relates to the treatment of infection by a microbe, which may be a bacterium or virus.

The compound of Formula (1) and carrier may be formulated into pharmaceutical compositions and dosage forms according to methods well known in the art. The pharmaceutical compositions of the present invention may be specially formulated for administration in liquid or solid form. In some embodiments, the pharmaceutical formulation is formulated for oral administration (e.g., as tablets, capsules, powders, granules, pastes, solutions, suspensions, drenches, or syrups); parenteral administration (e.g., by subcutaneous, intramuscular or intravenous injection as provided by, for example, a sterile solution or suspension); topical application (e.g., as a cream, ointment, or spray); intravaginal or intrarectal administration (e.g., as a pessary, cream or foam); sublingual or buccal administration; ocular administration; transdermal administration; or nasal administration. In some embodiments, the pharmaceutical composition is a liquid formulation designed for administration by injection.

The compound of Formula (1) can be incorporated in a pharmaceutical composition suitable for use as a medicament, for human or animal use. The pharmaceutical compositions may be, for instance, in an injectable formulation, a liquid, cream or lotion for topical application, an aerosol, a powder, granules, tablets, suppositories or capsules, such as for instance, enteric coated capsules, or the like. The pharmaceutical compositions may also be delivered in or on a lipid formulation, such as, for instance, an emulsion or a liposome preparation. The pharmaceutical compositions are preferably sterile, non-pyrogenic and isotonic preparations, optionally with one or more of the pharmaceutically acceptable additives listed below. Pharmaceutical compositions of the molecule are preferably stable compositions which may comprise one or more of the following: a stabilizer, a surfactant, preferably a nonionic surfactant, and optionally a salt and/or a buffering agent. The pharmaceutical composition may be in the form of an aqueous solution, or in a lyophilized form. The stabilizer may, for example, be an amino acid, such as for instance, glycine; or an oligosaccharide, such as for example, sucrose, tetralose, lactose, or a dextran. Alternatively, the stabilizer may be a sugar alcohol, such as for instance, mannitol; or a combination thereof. Preferably, the stabilizer or combination of stabilizers constitutes from about 0.1% to about 10% weight for weight of the molecule. The surfactant is preferably a nonionic surfactant, such as a polysorbate. Some examples of suitable surfactants include Tween® 20, Tween® 80; a polyethylene glycol or a polyoxyethylene polyoxypropylene glycol, such as Pluronic™ F-68 at from about 0.001% (w/v) to about 10% (w/v). The salt or buffering agent may be any salt or buffering agent, such as for example, sodium chloride, or sodium/potassium phosphate, respectively. Preferably, the buffering agent maintains the pH of the pharmaceutical composition in the range of about 5.5 to about 7.5. The salt and/or buffering agent is also useful to maintain the osmolality at a level suitable for administration to a human or an animal. Preferably, the salt or buffering agent is present at a roughly isotonic concentration of about 150 mM to about 300 mM.

The pharmaceutical composition comprising the molecule of Formula (1) may additionally contain one or more conventional additives. Some examples of such additives include a solubilizer such as for example, glycerol; an antioxidant such as, for example, benzalkonium chloride (a mixture of quaternary ammonium compounds, known as “quats”), benzyl alcohol, chloretone or chlorobutanol; anesthetic agent such as, for example, a morphine derivative; or an isotonic agent, such as described above. As a further precaution against oxidation or other spoilage, the pharmaceutical compositions may be stored under nitrogen or argon gas in vials sealed with impermeable stoppers.

The phrase “pharmaceutically acceptable” refers herein to those compounds, materials, compositions (e.g., acids or bases), and/or dosage forms which are, within the scope of sound medical judgment, suitable for administration to a subject. The phrase “pharmaceutically acceptable carrier,” as used herein, refers to a pharmaceutically acceptable vehicle, such as a liquid or solid filler, diluent, carrier, manufacturing aid (e.g., lubricant, talc magnesium, calcium or zinc stearate, or stearic acid), solvent, or encapsulating material, which serves to carry the therapeutic composition for administration to the subject. Each carrier should be “acceptable” in the sense of being compatible with the other ingredients of the formulation and physiologically safe to the subject. Any of the carriers known in the art can be suitable herein depending on the mode of administration.

Some examples of materials that can serve as pharmaceutically-acceptable carriers include water; isotonic saline; pH buffering agents; and sugars (e.g., lactose, glucose, sucrose, and oligosaccharides, such as sucrose, trehalose, lactose, or dextran). Other excipients, more typically used in solid dosage forms, may also be included, e.g., starches (e.g., corn and potato starch); cellulose and its derivatives (e.g., sodium carboxymethyl cellulose, ethyl cellulose and cellulose acetate); gelatin; talc; waxes; oils (e.g., peanut oil, cottonseed oil, safflower oil, sesame oil, olive oil, corn oil and soybean oil); glycols (e.g., ethylene glycol, propylene glycol, and polyethylene glycol); polyols (e.g., glycerin, sorbitol, and mannitol); esters (e.g., ethyl oleate and ethyl laurate); agar; and other non-toxic compatible substances employed in pharmaceutical formulations. If desired, certain sweetening and/or flavoring and/or coloring agents may be added. Other suitable excipients can be found in standard pharmaceutical texts, e.g., in “Rcmington's Pharmaceutical Sciences”, The Science and Practice of Pharmacy, 19th Ed. Mack Publishing Company, Easton, Pa., (1995).

In another aspect, the present disclosure is directed to methods of inhibiting papain-like protease (PLpro) activity in a subject by administering to the subject a therapeutically effective dosage of a compound of Formula (1) or sub-formula thereof to result in inhibition of PLpro activity in the subject. In particular embodiments, the PLpro activity is mediated by a coronavirus PLpro. In some embodiments, the method is more particularly a method of treating coronavirus infection in a subject by administering a therapeutically effective dosage of a compound of Formula (1) or sub-formula thereof to the subject to result in inhibition or prevention of one or more coronavirus symptoms in the subject. The subject being treated may be confirmed to have been infected with coronavirus or may display symptoms consistent with coronavirus infection or may be at risk for infection with coronavirus.

In another aspect, the compounds of Formula (1) or sub-formula thereof are useful in inhibiting papain-like protease activity, viral replicase complex formation, viral replication, and/or host immune pathway regulator cleavage of coronaviruses such as SARS-CoV, SARS-CoV-2, and related viruses. The method includes contacting the papain-like protease with a covalent inhibitor molecule having the generic structure of Formula (1) or sub-generic formula described herein.

In further aspects, the invention provides a method for treating or preventing a disease, disorder, or condition mediated by a coronavirus papain-like protease in a mammal in need thereof. The method includes administering to the mammal an inhibitor molecule having the generic structural Formula (1) or sub-generic formula described herein. The mammal is typically a human, but may be a farm animal, such as a goat, horse, pig, or cow; a pet animal, such as a dog or cat; a laboratory animal, such as a mouse, rat, or guinea pig; or a primate, such as a monkey, orangutan, ape, or chimpanzee.

The molecules of Formula (1) inhibit SARS-CoV-2 papain-like protease and thereby have a range of applications, such as therapeutic applications, because of the role that the papain-like protease (PLpro) plays in the physiology of SARS-CoV-2 replication and host infection. The reaction catalyzed by PLpro is the proteolytic cleavage of two viral polyproteins, pp1a and pp1ab, to produce the proteins Nsp1, Nsp2 and Nsp3. The first identified coronavirus PLpro was identified in the genome of a murine coronavirus (S. C. Baker et al., J. Virol., 63, 3693-3699 (1989). PLpro was later established as a therapeutic target (K. Ratia et al., Proc Natl Acad Sci USA 105, 16119-16124 (2008). A respiratory disease treatable with PLpro inhibitors within the scope of Formula (1) is coronavirus disease 2019 (COVID-19).

An effective amount of the molecule, typically in a pharmaceutical composition, may be administered to a human or an animal in need thereof by any of a number of well-known methods. For example, the molecule may be administered systemically or locally, such as by injection. The systemic administration of the molecule may be by intravenous, subcutaneous, intraperitoneal, intramuscular, intrathecal, or oral administration. An effective amount of a pharmaceutical composition of the invention is any amount that is effective to achieve its purpose. The effective amount, usually expressed in mg/kg, can be determined by routine methods during pre-clinical and clinical trials by those of skill in the art. Dosing is dependent on the severity and responsiveness of the infection being treated, with the course of treatment lasting from several days to several months, or until a cure is effected or a diminution of the disease state is achieved. Optimal dosing schedules can be calculated from measurements of drug accumulation in the body of the patient. The administering physician can determine optimum dosages, dosing methodologies, and repetition rates. In different embodiments, depending on these and other factors, a suitable dosage of the active ingredient may be precisely, at least, or no more than, for example, 1 mg, 10 mg, 50 mg, 100 mg, 200 mg, 300 mg, 400 mg, 500 mg, 600 mg, 700 mg, 800 mg, 900 mg, 1000 mg, 1200 mg, or 1500 mg, per 50 kg, 60 kg, or 70 kg adult, or a dosage within a range bounded by any of the foregoing exemplary dosages. Depending on these and other factors, the composition is administered in the indicated dosage by any suitable schedule, e.g., once, twice, or three times a day for a total treatment time of one, two, three, four, or five days, and up to, for example, one, two, three, or four weeks or months. The indicated dosage may alternatively be administered every two or three days, or per week. Alternatively, or in addition, the composition is administered until a desired change is evidenced.

In yet another aspect, a molecule having the generic structural Formula (1) described herein is also useful in the preparation and execution of screening assays for antiviral compounds. For example, the molecules are useful for identifying PLpro enzyme mutants that are resistant to these molecules, such that they can be used as screening tools for more potent antiviral compounds. The molecules can also be used to establish or determine the binding site of other antivirals to PLpro protease by, for example, competitive inhibition.

The ability of the molecule of Formula (1) or sub-formula thereof to inhibit PLpro may be demonstrated using a SARS-COV-2 PLpro enzyme inhibition assay using a fluorogenic peptide. For example, a recombinantly produced and purified SARS-COV-2 PLpro activity is assayed in the presence and absence of a molecule of Formula (1) or sub-formula thereof at varying concentrations and pre-incubation times, with Z-RLRGG-AMC or LRGG-AMC as substrate. These assays provide a means to compare the activities of molecules of Formula (1) to their prodrugs, salts, and formulations, and can be varied by those skilled in the art.

Inhibitors may be characterized by dispensing enzyme solution into wells, followed by inhibitor solution, and incubation for 30 min. Reactions may be initiated by adding substrate, and initial rates are then determined. IC₅₀ values at a single fixed time point (e.g., after 30-minute preincubation) may be determined by non-linear regression to the [Inhibitor] vs. normalized response. This assay can be used to measure IC₅₀ and adapted to obtain time dependent IC₅₀ values and thereby identify structure activity relationships. It can also be used as a high-throughput screen to identify new compounds with PLpro protease inhibition activity.

Time-dependent PLpro inhibition assays can be performed as described above, except that preincubation times are varied by adding the inhibitor to the enzyme at multiple specific time points. For each inhibitor concentration, initial rates are normalized and plotted against preincubation time. Non-linear regression is then performed to determine rate constants kobs for each concentration. These rate constants are then plotted against inhibitor concentration, and the data in the initial linear region can be fit to determine the slope, which is kinact/KI. Another such assay assesses the inhibition of PLpro-catalyzed cleavage of ubiquitin and/or ISG15 using ubiquitin-rhodamine or ISG15-CHOP2 as substrates. The CHOP2 reporter is inactive when linked to ISG15 but becomes catalytically active upon cleavage by PLpro. Thus, this coupled assay produces a signal upon cleavage of CHOP2 that quantitatively measures the activity of PLpro.

Another such assay can be performed using, for instance, Vero E6 cells to measure the cytopathic effect (CPE) protection for the 50% efficacy concentration (EC₅₀) and cytotoxicity (CC₅₀). Compounds can be evaluated at several concentrations using a luminescent cell viability assay to obtain EC₅₀ and CC₅₀ values. Briefly, Vero E6 TMPRSS ACE2 cells are grown, for example, to ˜90% confluency and treated for an amount of time, such as, for example, 1 hr, with compounds. Cells are then infected at an appropriate MOI (e.g., ˜0.1) with a viral isolate such as SARS-CoV-2 strain USA-WA1/2020. After some given time, for example, 48 h, the virally mediated CPE and cytotoxicity are assessed by measurement of live cells using the luminescent cell viability assay.

Examples have been set forth below for the purpose of illustration and to describe certain specific embodiments of the invention. However, the scope of this invention is not to be in any way limited by the examples set forth herein.

### EXAMPLES

### Overview

In describing the examples, chemical elements are identified in accordance with the Periodic Table of the Elements. Abbreviations and symbols used herein are in accordance with the common usage of such abbreviations and symbols by those skilled in the chemical arts. The following abbreviations are used herein:

| | |
|---|---|
| aq | aqueous |
| anh. | anhydrous |
| AMC | aminomethylcoumarin |
| CPE | cytopathic effect |
| DMSO | dimethylsulfoxide |
| CC₅₀ | 50% cytotoxic concentration |
| EC₅₀ | 50% effective concentration |
| IC₅₀ | 50% inhibition concentration |
| MOI | multiplicity of infection |
| SARS | Severe Acute Respiratory Syndrome |
| SI₅₀ | selectivity index at 50% |

Unless otherwise noted, all starting materials were obtained from commercial suppliers and used without further purification. Unless otherwise indicated, all temperatures are expressed in ° C. (degrees Celsius). Unless otherwise indicated, all reactions were conducted under an inert atmosphere at ambient temperature.

All temperatures are given in degrees Celsius. All solvents are of the highest available purity and all reactions were run under anhydrous conditions in an argon (Ar) or nitrogen (N₂) atmosphere where necessary.

The following examples illustrate the invention. These examples are not intended to limit the scope of the present invention, but rather to provide guidance to the skilled artisan to prepare and use the compounds, compositions, and methods of the present invention.

### Synthesis and Characterization of Compounds of Formula (1)

Selected compounds of Formula (1) were synthesized in accordance with the following synthetic scheme:

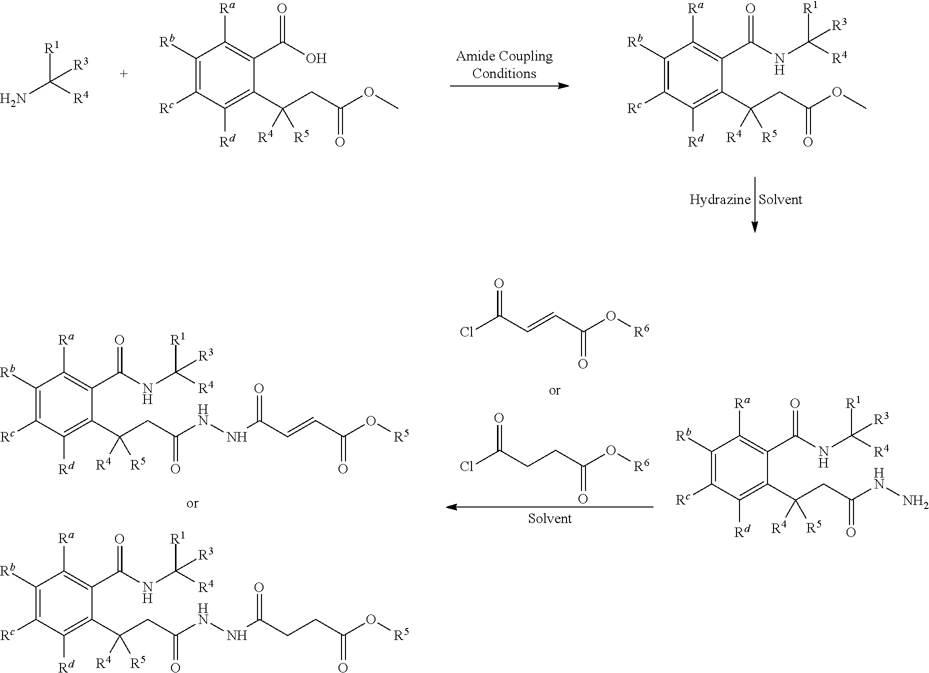

### Experimental Details

### Synthesis of Compound 1.2 (NEU-7768)

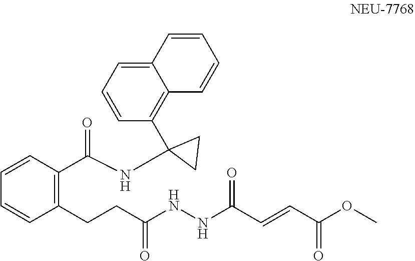 [SMILES: COC(=O)/C=C/C(=O)NNC(=O)CCc1ccccc1C(=O)NC1(c2cccc3ccccc23)CC1]

### General Schematic for Synthesis of NEU-7768

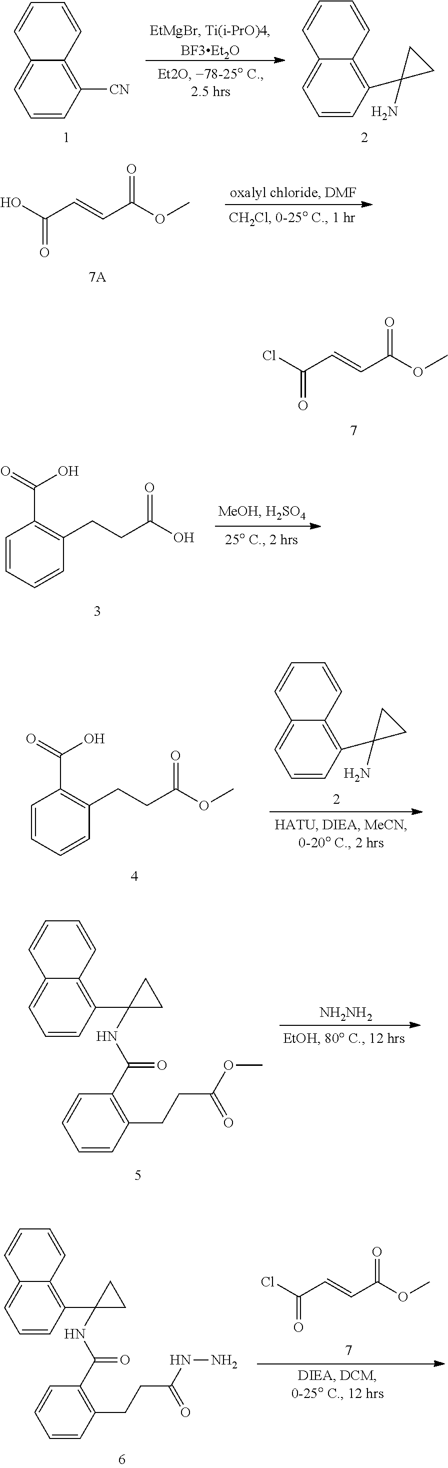

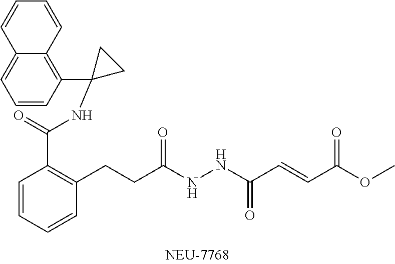 [SMILES: COC(=O)/C=C/C(=O)NNC(=O)CCc1ccccc1C(=O)NC1(c2cccc3ccccc23)CC1]

### Synthesis of Compound 2

To a solution of compound 1 (2 g, 13.06 mmol, 1 eq) in Et₂O (200 mL) was added EtMgBr (3 M, 8.70 mL, 2 eq) and Ti(i-PrO)4 (5.10 g, 14.36 mmol, 5.30 mL, 80% purity, 1.1 eq) at −78° C. for 0.5 hrs. When the solution was warmed to 25° C. for 1 hr, BF₃·Et₂O (3.71 g, 26.11 mmol, 3.22 mL, 2 eq) was added. The mixture was stirred at 25 for 1 hr. 1 N HCl (100 mL) and TBME (100 mL) were added in the reaction. NaOH (10% aq 100 mL) was added to the water phase and the mixture was extracted with EtOAc (300 mL×3). The combined organic layers were dried Na₂SO₄, filtered, and concentrated under reduced pressure to give compound 2 (730 mg, 3.35 mmol, 25.63% yield, 84% purity) as brown oil which was confirmed by ¹H NMR.

¹H NMR: (400 MHz, chloroform-d) δ 8.48 (d, J=8.4 Hz, 1H), 7.93 (d, J=8.0 Hz, 1H), 7.80 (d, J=8.0 Hz, 1H), 7.65-7.60 (m, 1H), 7.58-7.53 (m, 2H), 7.47-7.43 (m, 1H), 1.27-1.22 (m, 2H), 1.10-1.06 (m, 2H).

### General Procedure for Preparation of Compound 4

To a solution of compound 3 (2 g, 10.30 mmol, 1 eq) in MeOH (20 mL) was added H₂SO₄ (736.00 mg, 7.35 mmol, 0.4 mL, 98% purity, 7.14e-1 eq). The mixture was stirred at 25° C. for 2 hrs. Then the solvent was removed, and 10 ml of water and 1M NaOH solution were added. The obtained mixture was stirred and adjusted to pH˜8-9 with the saturated NaHCO₃ solution. The solution was extracted with EtOAc (30 mL×3), and the organic phase was dried to give a residue. The residue was purified by flash silica gel chromatography (ISCO®; 12 g SepaFlash® Silica Flash Column, Eluent of 18-20% Ethyl acetate/Petroleum ether gradient @ 50 mL/min) to give compound 4 (1.4 g, 6.50 mmol, 63.13% yield, 96.7% purity) as white solid which was confirmed by ¹H NMR.

¹H NMR: (400 MHz, chloroform-d) δ 8.08 (dd, J=1.6, 8.3 Hz, 1H), 7.54-7.47 (m, 1H), 7.36-7.32 (m, 2H), 3.68 (s, 3H), 3.36 (t, J=7.6 Hz, 2H), 2.75-2.70 (m, 2H).

### General Procedure for Preparation of Compound 5

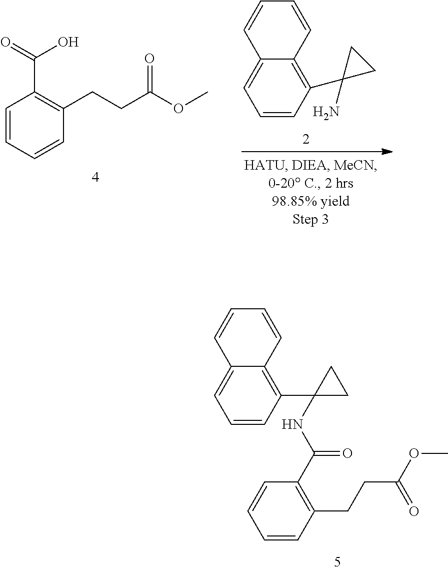

To a solution of compound 4 (500 mg, 2.40 mmol, 1 eq) in MeCN (5 mL) was added HATU (1.37 g, 3.60 mmol, 1.5 eq) at 0° C. for 5 min. Then 1-(1-naphthyl)cyclopropanamine (660.09 mg, 3.60 mmol, 1.5 eq) in MeCN (5 mL) was added DIEA (931.10 mg, 7.20 mmol, 1.25 mL, 3 eq) to the mixture. The mixture was stirred at 25° C. for 3 hrs. After cooling to room temperature, the reaction mixture was concentrated to give a residue. The residue was diluted with H₂O (30 mL) and extracted with EtOAc (30 mL×3). The combined organic layers were dried over Na₂SO₄, filtered and the filtrate was concentrated to give a residue. The residue was purified by flash silica gel chromatography (ISCO®; 12 g SepaFlash® Silica Flash Column, Eluent of 25-30% Ethyl acetate/Petroleum ether gradient @ 60 mL/min). to give compound 5 (900 mg, 2.37 mmol, 98.85% yield, 98.5% purity) as a yellow oil which was confirmed by LCMS and ¹H NMR.

LCMS Rt=0.766, M+1=374.2, 98.5% purity.

¹H NMR: (400 MHz, DMSO-d6) δ 9.21 (s, 1H), 8.65 (d, J=8.4 Hz, 1H), 7.93 (d, J=7.6 Hz, 1H), 7.83 (t, J=6.4 Hz, 2H), 7.59-7.50 (m, 2H), 7.46 (t, J=7.6 Hz, 1H), 7.30-7.26 (m, 1H), 7.20-7.14 (m, 2H), 7.04 (d, J=7.6 Hz, 1H), 3.52-3.47 (m, 3H), 2.74-2.67 (m, 2H), 2.28 (t, J=8.0 Hz, 2H), 1.40-1.33 (m, 2H), 1.21-1.18 (m, 2H).

### General Procedure for Preparation of Compound 6

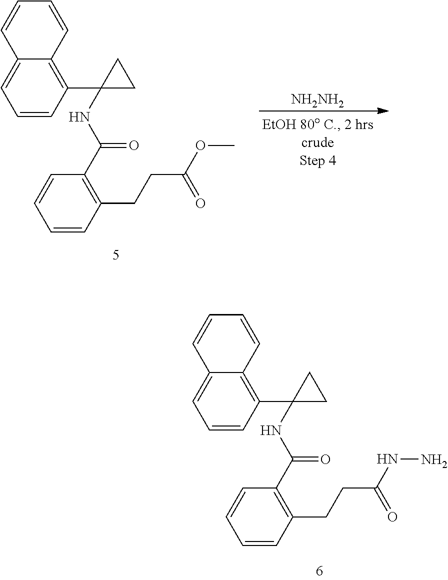 [SMILES: COC(=O)CCc1ccccc1C(=O)NC1(c2cccc3ccccc23)CC1 ; "NH2NH2" ; NNC(=O)CCc1ccccc1C(=O)NC1(c2cccc3ccccc23)CC1]

To a solution of Compound 5 (400 mg, 1.07 mmol, 1 eq) in EtOH (10 mL) was added NH₂NH₂·H₂O (804.30 mg, 16.07 mmol, 780.88 μL, 15 eq). The mixture was stirred at 80° C. for 12 hrs. After cooling to room temperature, the reaction mixture was concentrated to give a residue. The residue was diluted with H₂O (15 mL) and extracted with EtOAc (20 mL×3). The combined organic layers were dried over Na₂SO₄, filtered and the filtrate was concentrated to give compound 6 (500 mg, crude) as yellow oil which was confirmed by ¹H NMR.

¹H NMR. (400 MHz, DMSO-d6) δ 9.30-9.24 (m, 1H), 8.99-8.77 (m, 2H), 8.66 (d, J=8.4 Hz, 1H), 7.93 (d, J=7.2 Hz, 1H), 7.86-7.79 (m, 2H), 7.61-7.55 (m, 1H), 7.55-7.50 (m, 1H), 7.46 (dd, J=7.2, 8.0 Hz, 1H), 7.30-7.23 (m, 1H), 7.19-7.09 (m, 2H), 6.99 (dd, J=1.2, 7.6 Hz, 1H), 4.13 (br s, 2H), 2.76-2.68 (m, 2H), 2.21 (t, J=7.8 Hz, 2H), 1.42-1.36 (m, 2H), 1.19-1.15 (m, 2H).

### General Procedure for Preparation of Compound 7

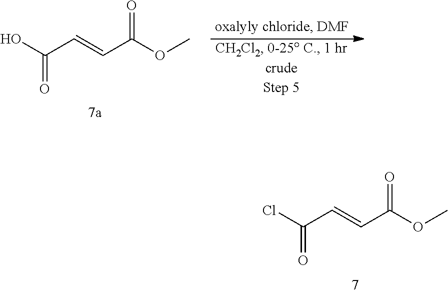

Compound 7A (1 g, 7.69 mmol, 1 eq) was dissolved in CH₂Cl₂ (10 mL). After DMF (10 mg, 136.81 μmol, 10.53 μL, 1.78e-2 eq) was added, the solution was cooled to 0° C. and oxalyl dichloride (1.07 g, 8.46 mmol, 740.12 μL, 1.1 eq) was added dropwise. The resulting mixture was stirred at 0° C. for 5 min then at 25° C. for 3 hrs. After cooling to room temperature, the reaction mixture was concentrated to give compound 7 (950 mg, crude) as white solid.

### General Procedure for Preparation of Compound NEU-7768 (Compound 1.2)

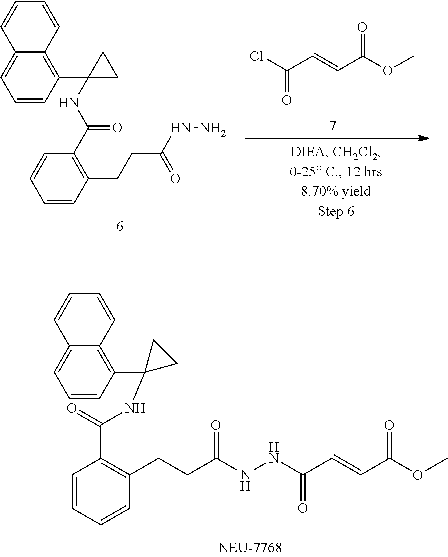

To a solution of compound 6 (450 mg, 1.20 mmol, 1 eq) in CH₂Cl₂, (5 mL) was added DIEA (186.88 mg, 1.45 mmol, 251.87 μL, 1.2 eq) and methyl (E)-4-chloro-4-oxo-but-2-enoate (268.49 mg, 1.81 mmol, 1.5 eq) at 0° C. The mixture was stirred at 25° C. for 12 hrs. After cooling to room temperature, water was added into the reaction system and precipitation was formed. The solid was collected by filtration, washed with CH₂Cl₂ (15 mL×3) and dried under reduced pressure to give a residue. The residue was triturated with CH₂Cl₂ (20 mL). The resulting solid was collected by filtration, washed with CH₂Cl₂ (10 mL) and dried to give NEU-7768 (52.5 mg, 104.85 μmol, 8.70% yield, 96.97% purity) as white solid which was confirmed by LCMS and ¹H NMR.

LCMS, Rt=2.398, M+1=486.1, 96.97% purity.

¹H NMR. (400 MHz, DMSO-d6) δ 10.56 (br s, 1H), 10.19-10.14 (m, 1H), 9.23 (s, 1H), 8.65 (d, J=8.4 Hz, 1H), 7.93 (d, J=7.6 Hz, 1H), 7.82 (dd, J=3.2, 7.6 Hz, 2H), 7.61-7.56 (m, 1H), 7.55-7.50 (m, 1H), 7.49-7.43 (m, 1H), 7.31-7.26 (m, 1H), 7.25-7.19 (m, 1H), 7.17-7.11 (m, 1H), 7.09-7.03 (m, 1H), 6.98 (d, J=6.6 Hz, 1H), 6.72-6.64 (m, 1H), 3.75-3.73 (m, 3H), 2.78 (br t, J=8.0 Hz, 2H), 2.43-2.32 (m, 2H), 1.38 (s, 2H), 1.21-1.13 (m, 2H).

### Synthesis of Compound 2.2 (NEU-7769)

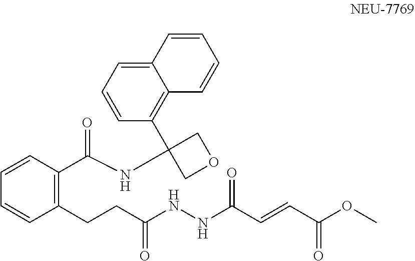 [SMILES: COC(=O)/C=C/C(=O)NNC(=O)CCc1ccccc1C(=O)NC1(c2cccc3ccccc23)COC1]

### General Schematic for Synthesis of Compound 2.2 (NEU-7769)

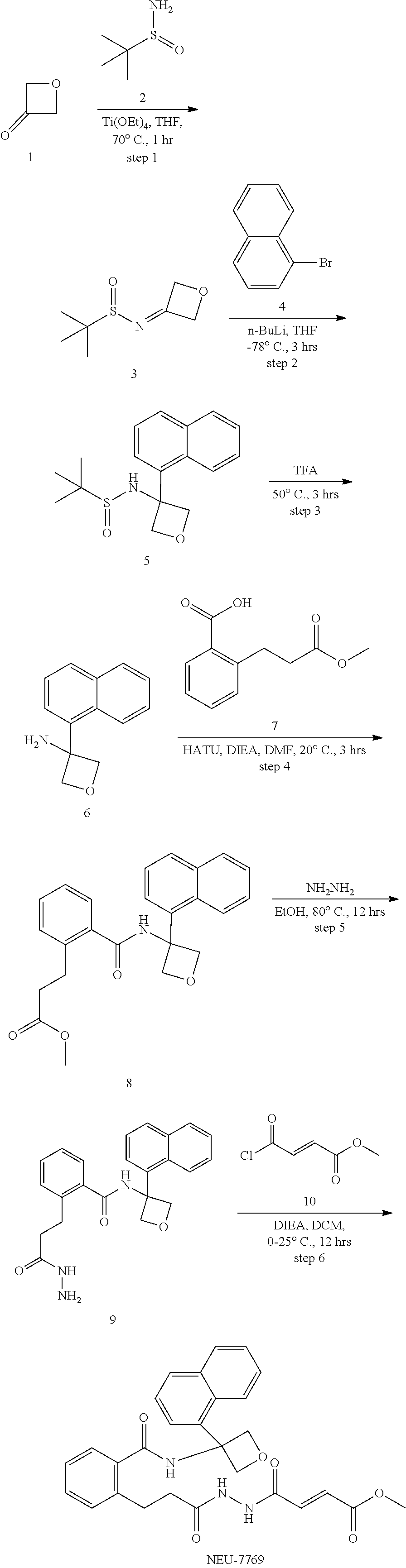

### General Procedure for Preparation of Compound 3

To a solution of compound 1 (10 g, 138.77 mmol, 1 eq) and 2-methylpropane-2-sulfinamide (16.82 g, 138.77 mmol, 1 eq) in THF (100 mL) was added Ti(OEt)₄ (63.31 g, 277.54 mmol, 57.55 mL, 2 eq). The mixture was stirred at 70° C. for 1 hr. The resultant mixture was filtered and the filter cake was rinsed with H₂O (50 mL×3) and the aqueous phase was extracted with EtOAc (100 mL×3). The combined organic layers were dried over Na₂SO₄, filtered and the filtrate was concentrated to give a residue. The residue was purified by flash silica gel chromatography (ISCO₂; 80 g SepaFlash Silica Flash Column, eluent of 20-30% ethyl acetate/petroleum ether gradient @ 100 mL/min) to give compound 3 (3.5 g, crude) which was a yellow oil that was confirmed by LCMS.

LCMS: Rt=0.407 min, [M+1]=176.2, 88.47% purity

### General Procedure for Preparation of Compound 5

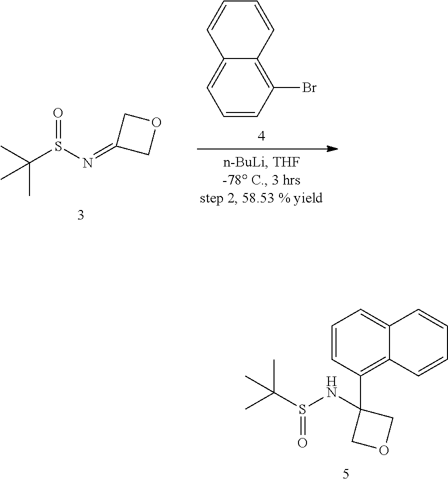 [SMILES: CC(C)(C)S(=O)N=C1COC1 ; Brc1cccc2ccccc12 ; CC(C)(C)S(=O)NC1(c2cccc3ccccc23)COC1]

To a solution of 1-bromonaphthalene (4.08 g, 19.69 mmol, 2.74 mL, 1.5 eq) in THF (20 mL) was added n-BuLi (2.5 M, 7.35 mL, 1.4 eq) at −78° C. for 1 hr. Then compound 3 (2.3 g, 13.12 mmol, 1 eq) was added into the mixture. The mixture was stirred at −78° C. for 2 hrs. The residue was diluted with sat. NH₄Cl (30 mL) and the aqueous phase was extracted with EtOAc (30 mL×3). The combined organic layers were dried over Na₂SO₄, filtered and the filtrate was concentrated to give a residue. The residue was purified by flash silica gel chromatography (ISCO₂; 40 g SepaFlash Silica Flash Column, eluent of 10-20% ethyl acetate/petroleum ether gradient @ 50 mL/min) to give compound 5 (2.5 g, 7.68 mmol, 58.53% yield, 93.23% purity) as a white solid which was confirmed by ¹H NMR; LCMS.

LCMS: Rt=0.631 min, [M+1]=304.1, 93.23% purity

¹H NMR (400 MHz, DMSO-d6) δ 7.88-8.00 (m, 2H), 7.46-7.61 (m, 5H), 6.55 (s, 1H), 5.44 (br d, J=6.8 Hz, 1H), 5.32 (d, J=6.4 Hz, 1H), 5.11 (d, J=6.8 Hz, 1H), 4.92 (br d, J=6.4 Hz, 1H), 0.93 (s, 9H).

### General Procedure for Preparation of Compound 6

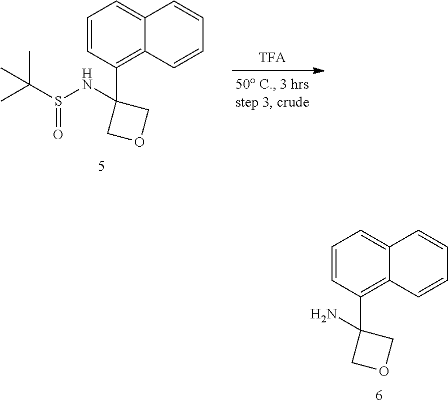 [SMILES: CC(C)(C)S(=O)NC1(c2cccc3ccccc23)COC1 ; NC1(c2cccc3ccccc23)COC1]

To a solution of compound 5 (2 g, 6.59 mmol, 1 eq) in TFA (10 mL). The mixture was stirred at 50° C. for 3 hrs. After cooling to room temperature, the reaction mixture was concentrated and lyophilized to give compound 6 (2 g, crude) was a yellow solid which was confirmed by ¹H NMR.

¹H NMR (400 MHz, DMSO-d6) δ 9.24 (br s, 2H), 8.04-8.10 (m, 2H), 7.63 (dt, J=7.4, 2.8 Hz, 3H), 5.29 (d, J=7.8 Hz, 2H), 5.19 (d, J=7.8 Hz, 2H), 1.37 (s, 1H), 1.22 (s, 1H).

### General Procedure for Preparation of Compound 8

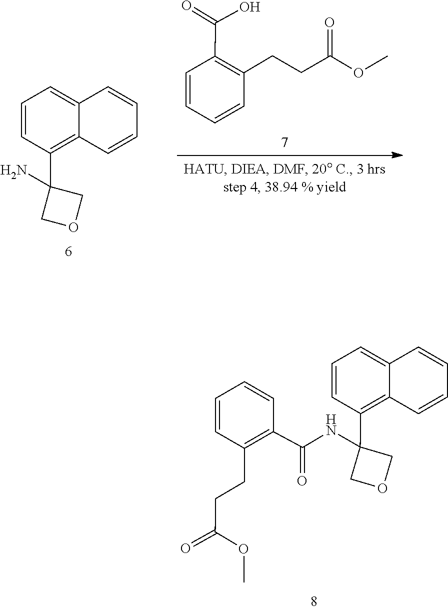 [SMILES: NC1(c2cccc3ccccc23)COC1 ; COC(=O)CCc1ccccc1C(=O)O ; COC(=O)CCc1ccccc1C(=O)NC1(c2cccc3ccccc23)COC1]

To a solution of 3-(1-naphthyl)oxetan-3-amine (717.72 mg, 3.60 mmol, 1.5 eq) in MeCN (2.5 mL) was added HATU (1.37 g, 3.60 mmol, 1.5 eq) at 0° C. for 5 min. Then compound 6 (0.5 g, 2.40 mmol, 1 eq) in MeCN (5 mL) and DIEA (931.08 mg, 7.20 mmol, 1.25 mL, 3 eq) were added into the mixture. The mixture was stirred at 0° C. for 3 hrs. The residue was diluted with H₂O (30 mL) and the aqueous phase was extracted with EtOAc (30 mL×3). The combined organic layers were dried over Na₂SO₄, filtered and the filtrate was concentrated to give a residue. The residue was purified by flash silica gel chromatography (ISCO₂; 20 g SepaFlash Silica Flash Column, eluent of 20˜25% ethyl acetate/petroleum ether gradient @ 70 mL/min) to give compound 8 (410 mg, 935.08 μmol, 38.94% yield, 88.82% purity) as a yellow oil which was confirmed by ¹HNMR and LCMS.

LCMS: Rt=0.690 min, [M+1]=390.2, 88.82% purity

¹H NMR: (400 MHz, DMSO-d6) δ 9.84 (s, 1H), 7.96-8.00 (m, 1H), 7.88 (d, J=8.2 Hz, 1H), 7.74-7.80 (m, 2H), 7.50-7.54 (m, 3H), 7.29-7.33 (m, 1H), 7.19-7.23 (m, 2H), 7.09-7.13 (m, 1H), 5.21-5.26 (m, 4H), 3.47 (s, 3H), 2.69 (s, 2H), 2.18-2.22 (m, 2H).

### General Procedure for Preparation of Compound 9

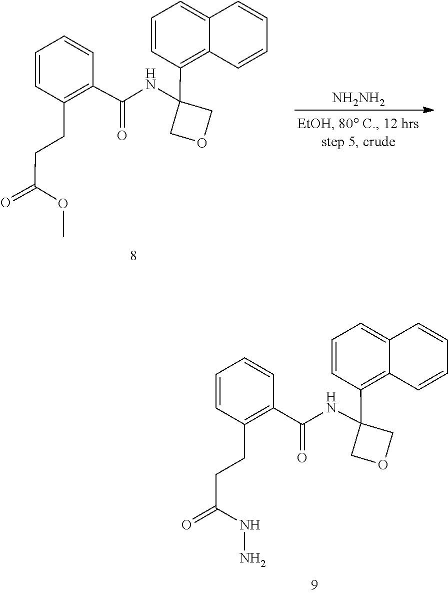 [SMILES: COC(=O)CCc1ccccc1C(=O)NC1(c2cccc3ccccc23)COC1 ; "NH2NH2" ; NNC(=O)CCc1ccccc1C(=O)NC1(c2cccc3ccccc23)COC1]

To a solution of compound 8 (0.36 g, 924.40 μmol, 1 eq) in EtOH (5 mL) was added NH₂NH₂·H₂O (694.13 mg, 13.87 mmol, 673.92 μL, 15 eq). The mixture was stirred at 80° C. for 12 hrs. The reaction mixture was concentrated to give a residue. The residue was diluted with H₂O (30 mL) and the aqueous phase was extracted with EtOAc (30 mL×3). The combined organic layers were dried over Na₂SO₄, filtered and the filtrate was concentrated to give compound 9 (0.34 g, crude) as a yellow solid which was confirmed by ¹H NMR.

¹H NMR: (400 MHz, DMSO-d6) δ 9.94 (s, 1H), 8.86 (br s, 1H), 7.96-8.01 (m, 1H), 7.88 (d, J=8.2 Hz, 1H), 7.80 (br d, J=7.2 Hz, 2H), 7.51-7.56 (m, 3H), 7.28-7.33 (m, 1H), 7.14-7.23 (m, 2H), 7.00-7.06 (m, 1H), 5.20-5.30 (m, 4H), 4.14 (br s, 2H), 2.78 (t, J=7.6 Hz, 2H), 2.22 (t, J=7.6 Hz, 2H).

### General Procedure for Preparation of NEU-7769

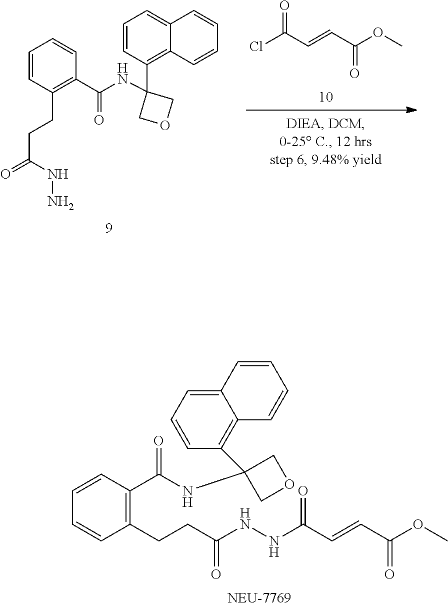

To a solution of compound 9 (250 mg, 641.94 μmol, 1 eq) in CH₂Cl₂ (5 mL) was added DIEA (99.56 mg, 770.32 μmol, 134.18 μL, 1.2 eq) and methyl (E)-4-chloro-4-oxo-but-2-enoate (143.03 mg, 962.91 μmol, 1.5 eq) at 0° C. The mixture was stirred at 25° C. for 1 hr. The reaction mixture was concentrated to give a residue. The residue was diluted with 1 N HCl (30 mL) and the aqueous phase was extracted with EtOAc (30 mL×3). The combined organic layers were dried over Na₂SO₄, filtered and the filtrate was concentrated to give a residue. The residue was triturated with CH₂Cl₂ (30 mL). The resulting solid was collected by filtration to give NEU-7769 (0.064 g, 121.75 μmol, 9.48% yield, 95.41% purity) as a white solid which was confirmed by ¹HNMR and LCMS.

LCMS: Rt=2.254 min, [M+1]=502.2, 95.41% purity

¹H NMR (400 MHz, DMSO-d6) δ 10.55 (br s, 1H), 10.05-10.22 (m, 1H), 9.86 (s, 1H), 7.96-8.01 (m, 1H), 7.88 (d, J=8.4 Hz, 1H), 7.80 (br t, J=6.4 Hz, 2H), 7.50-7.56 (m, 3H), 7.24-7.34 (m, 2H), 7.18 (td, J=7.4, 1.2 Hz, 1H), 7.00-7.09 (m, 2H), 6.68 (d, J=15.6 Hz, 1H), 5.20-5.28 (m, 4H), 2.83 (br t, J=7.8 Hz, 2H), 2.38 (br t, J=7.8 Hz, 2H).

### General Schematic for Synthesis of Compound 3.2 (NEU 7770)

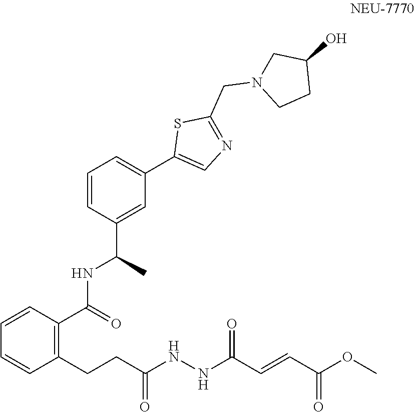 [SMILES: COC(=O)/C=C/C(=O)NNC(=O)CCc1ccccc1C(=O)N[C@H](C)c1cccc(-c2cnc(CN3CC[C@H](O)C3)s2)c1]

### General Schematic for Synthesis of Compound 3.2 (NEU 7770)

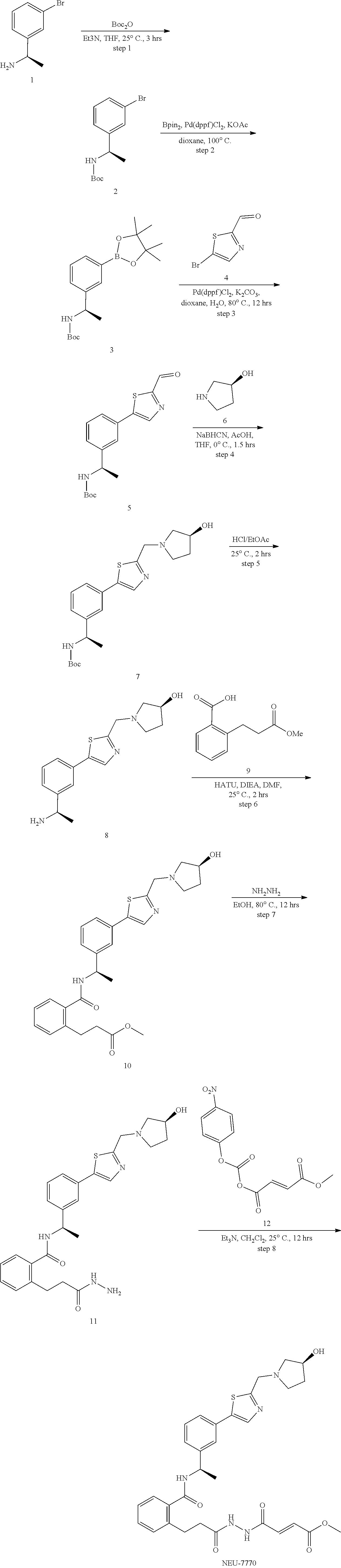

### General Procedure for Preparation of Compound 2

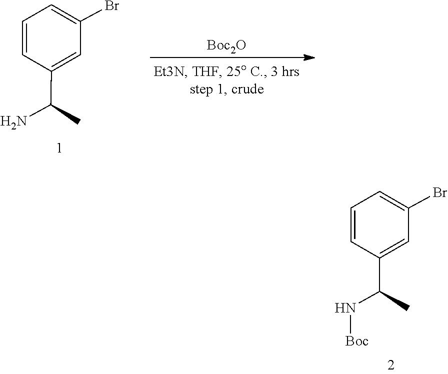

To a solution of compound 1 (4.5 g, 22.49 mmol, 1 eq) in THF (45 mL) was added Et₃N (3.41 g, 33.74 mmol, 4.70 mL, 1.5 eq) and Boc₂O (5.89 g, 26.99 mmol, 6.20 mL, 1.2 eq). The mixture was stirred at 25° C. for 3 hrs. The reaction mixture was concentrated to give a residue. The residue was diluted with H₂O (50 mL) and the aqueous phase was extracted with EtOAc (80 mL×3). The combined organic layers were dried over Na₂SO₄, filtered and the filtrate was concentrated to give a residue. The residue was purified by flash silica gel chromatography (ISCO₂; 20 g SepaFlash Silica Flash Column, eluent of 10-15% ethyl acetate/petroleum ether gradient @ 50 mL/min) to give compound 2 (7.8 g, crude) as a white oil which was confirmed by ¹HNMR.

¹H NMR (400 MHz, DMSO-d6) δ 7.49 (s, 1H), 7.36-7.47 (m, 2H), 7.25-7.29 (m, 2H), 4.48-4.66 (m, 1H), 1.47 (s, 2H), 1.36 (s, 7H), 1.28 (d, J=7.0 Hz, 3H)

### General Procedure for Preparation of Compound 3

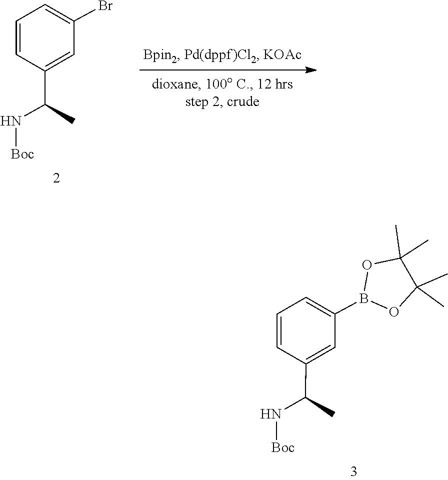 [SMILES: C[C@@H](NC(=O)OC(C)(C)C)c1cccc(Br)c1 ; C[C@@H](NC(=O)OC(C)(C)C)c1cccc(B2OC(C)(C)C(C)(C)O2)c1]

To a solution of compound 2 (6.3 g, 20.99 mmol, 1 eq) in dioxane (65 mL) was added KOAc (3.09 g, 31.48 mmol, 1.50 eq), 4,4,5,5-tetramethyl-2-(4,4,5,5-tetramethyl-1,3,2-dioxaborolan-2-yl)-1,3,2-dioxaborolane (5.33 g, 20.99 mmol, 1 eq) and [1,1′-bis(diphenylphosphino)ferrocene]dichloropalladium(II) (856.92 mg, 1.05 mmol, 0.05 eq). The mixture was stirred at 100° C. for 12 hrs. After cooling to room temperature, the reaction mixture was concentrated to give a residue. The residue was diluted with H₂O (30 mL) and the aqueous phase was extracted with EtOAc (30 mL×3). The combined organic layers were dried over Na₂SO₄, filtered and the filtrate was concentrated to give a residue. The resultant mixture was filtered and the filter cake was rinsed with PE (30 mL×3). Then the combined filtrates were concentrated under reduced pressure to give compound 3 (7 g, crude) was a yellow oil which was confirmed by ¹HNMR.

¹H NMR (400 MHz, DMSO-d6) δ 7.62 (s, 1H), 7.51 (d, J=7.2 Hz, 1H), 7.40 (br d, J=7.6 Hz, 1H), 7.29-7.33 (m, 1H), 4.55-4.62 (m, 1H), 3.57 (s, 1H), 1.36 (s, 9H), 1.16 (s, 12H), 1.07 (s, 3H).

### General Procedure for Preparation of Compound 5

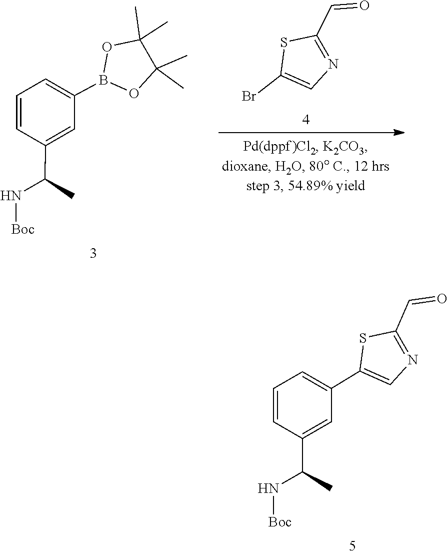 [SMILES: C[C@@H](NC(=O)OC(C)(C)C)c1cccc(B2OC(C)(C)C(C)(C)O2)c1 ; O=Cc1ncc(Br)s1 ; C[C@@H](NC(=O)OC(C)(C)C)c1cccc(-c2cnc(C=O)s2)c1]

To a solution of compound 3 (5 g, 14.40 mmol, 1 eq) and 5-bromothiazole-2-carbaldehyde (2.49 g, 12.96 mmol, 0.9 eq) in dioxane (60 mL) and H₂O (10 mL) was added [1,1′-bis(diphenylphosphino)ferrocene]dichloropalladium(II) (105.36 mg, 143.99 μmol, 0.01 eq) and K₂CO₃ (3.98 g, 28.80 mmol, 2 eq). The mixture was stirred at 80° C. for 12 hrs. The resultant mixture was filtered and the filter cake was rinsed with EtOAc (5 mL×3). Then the combined filtrates were diluted with H₂O (50 mL) and the aqueous phase was extracted with EtOAc (80 mL×3). The combined organic layers were dried over Na₂SO₄, filtered and the filtrate was concentrated to give a residue. The residue was purified by column chromatography (SiO₂, petroleum ether/ethyl acetate=10/1 to 5/1) to give compound 5 (2.9 g, 7.90 mmol, 54.89% yield, 90.60% purity) as a yellow solid which was confirmed by ¹HNMR; LCMS.

LCMS: Rt=0.766 min, [M+1]=333.1, 90.60%% purity.

¹H NMR (400 MHz, DMSO-d6) 9.93 (s, 1H), 8.66 (s, 1H), 7.75 (s, 1H), 7.71 (br d, J=7.8 Hz, 1H), 7.46 (br t, J=7.8 Hz, 2H), 7.39-7.42 (m, 1H), 4.63-4.76 (m, 1H), 1.37 (s, 9H), 1.33 (br d, J=7.2 Hz, 3H).

### General Procedure for Preparation of Compound 7

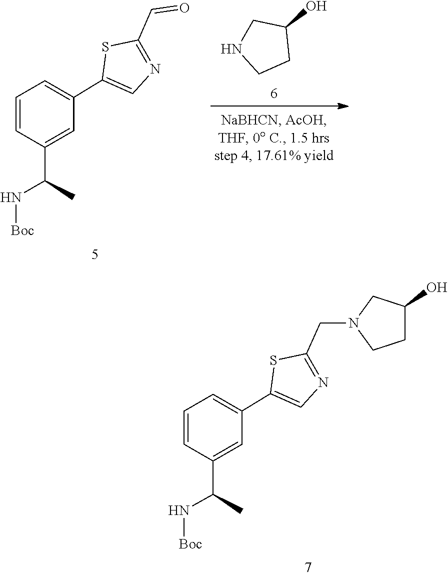 [SMILES: C[C@@H](NC(=O)OC(C)(C)C)c1cccc(-c2cnc(C=O)s2)c1 ; O[C@H]1CCNC1 ; C[C@@H](NC(=O)OC(C)(C)C)c1cccc(-c2cnc(CN3CC[C@H](O)C3)s2)c1]

To a solution of compound 5 (2.9 g, 8.72 mmol, 1 eq) and (3S)-pyrrolidin-3-ol (760.03 mg, 8.72 mmol, 703.74 μL, 1 eq) in THF (30 mL) was added AcOH (523.88 mg, 8.72 mmol, 498.93 μL, 1 eq) at 0° C. for 0.5 hr. Then NaBH₃CN (1.64 g, 26.17 mmol, 3 eq) was added to the mixture. The mixture was stirred at 0° C. for 1 hr. The residue was diluted with H₂O (40 mL) and the aqueous phase was extracted with EtOAc (60 mL×3). The combined organic layers were dried over Na₂SO₄, filtered and the filtrate was concentrated to give a residue. The residue was purified by prep-HPLC (neutral condition; column: Waters Xbridge BEH C18 250×70 mm×10 m; mobile phase: [water(NH₄HCO₃)-ACN]; B %: 25%-55%, 20 min) to give compound 7 (0.62 g, 1.54 mmol, 17.61% yield, 100% purity) as a white solid which was confirmed by ¹HNMR; LCMS.

LCMS: Rt=0.505 min, [M+1]=404.2, 100.00% purity.

¹H NMR (400 MHz, DMSO-d6) δ 8.03 (s, 1H), 7.55 (s, 1H), 7.50 (br d, J=7.8 Hz, 1H), 7.44 (br d, J=8.2 Hz, 1H), 7.36 (t, J=7.6 Hz, 1H), 7.26 (d, J=7.8 Hz, 1H), 4.78 (d, J=4.4 Hz, 1H), 4.64 (br t, J=7.4 Hz, 1H), 4.24 (dt, J=6.6, 3.5 Hz, 1H), 3.92 (d, J=2.2 Hz, 2H), 2.88 (dd, J=9.8, 6.1 Hz, 1H), 2.74 (d, J=8.4 Hz, 1H), 2.59-2.65 (m, 1H), 2.46-2.49 (m, 1H), 1.96-2.04 (m, 1H), 1.56-1.63 (m, 1H), 1.37 (s, 9H), 1.32 (d, J=7.2 Hz, 3H).

### General Procedure for Preparation of Compound 8

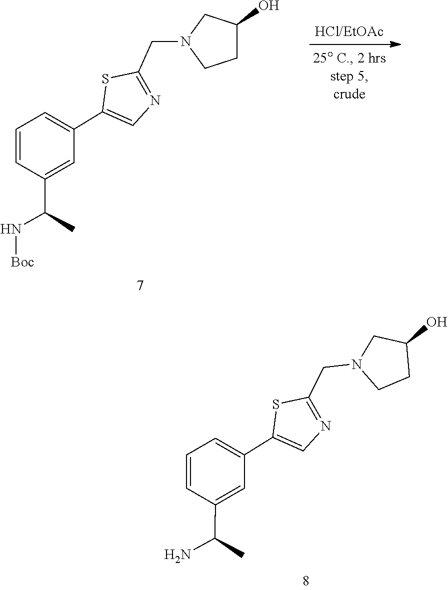 [SMILES: C[C@@H](NC(=O)OC(C)(C)C)c1cccc(-c2cnc(CN3CC[C@H](O)C3)s2)c1 ; C[C@@H](N)c1cccc(-c2cnc(CN3CC[C@H](O)C3)s2)c1]

To a solution of compound 7 (0.62 g, 1.54 mmol, 1 eq) in HCl/EtOAc (4 M, 3.84 mL, 10 eq). The mixture was stirred at 25° C. for 2 hrs. The solid was collected by filtration, rinsed with EtOAc (5 mL×3) and dried under reduced pressure to give compound 8 (0.54 g, crude) as a white solid which was confirmed by ¹H NMR.

¹H NMR (400 MHz, DMSO-d6) δ 8.39 (s, 1H), 8.01 (br d, J=9.4 Hz, 1H), 7.70 (br d, J=7.2 Hz, 1H), 7.49-7.56 (m, 2H), 4.79-4.88 (m, 4H), 4.40-4.48 (m, 2H), 3.62 (br dd, J=11.8, 5.8 Hz, 2H), 3.38 (br s, 2H), 3.17 (br d, J=11.8 Hz, 1H), 1.77-2.12 (m, 2H), 1.56 (d, J=6.8 Hz, 3H).

### General Procedure for Preparation of Compound 10

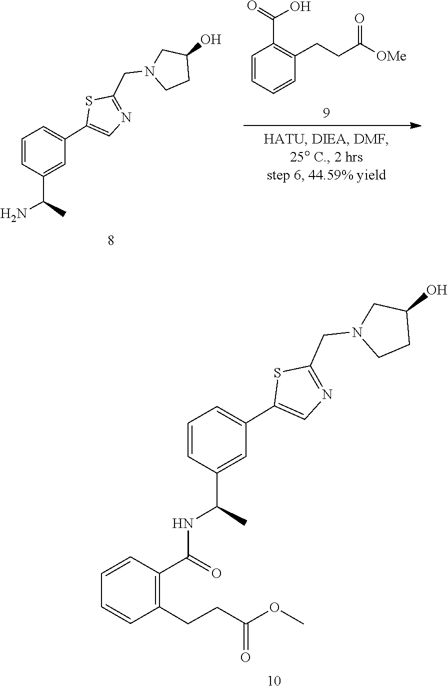 [SMILES: C[C@@H](N)c1cccc(-c2cnc(CN3CC[C@H](O)C3)s2)c1 ; COC(=O)CCc1ccccc1C(=O)O ; COC(=O)CCc1ccccc1C(=O)N[C@H](C)c1cccc(-c2cnc(CN3CC[C@H](O)C3)s2)c1]

To a solution of 2-(3-methoxy-3-oxo-propyl)benzoic acid (247.03 mg, 1.19 mmol, 1.2 eq) in DMF (6 mL) was added HATU (563.91 mg, 1.48 mmol, 1.5 eq) at 0° C. for 5 min. Then compound 8 (0.3 g, 988.72 μmol, 1 eq) in DMF (6 mL) and DIEA (383.36 mg, 2.97 mmol, 516.65 μL, 3 eq) were added to the mixture. The mixture was stirred at 25° C. for 2 hrs. The residue was diluted with H₂O (20 mL) and the aqueous phase was extracted with EtOAC (30 mL×3). The combined organic layers were dried over Na₂SO₄, filtered and the filtrate was concentrated to give a residue. The residue was purified by prep-HPLC (neutral condition; column: Phenomenex C18 80×40 mm×3 um; mobile phase: [water(NH₄HCO₃)-ACN]; B %: 30%-60%, 8 min) to give compound 10 (0.22 g, 440.83 μmol, 44.59% yield, 98.91% purity) as a white solid which was confirmed by ¹H NMR; LCMS.

LCMS: Rt=0.508 min, [M+1]=494.2, 98.91% purity.

¹H NMR (400 MHz, DMSO-d6) δ 8.85 (d, J=8.2 Hz, 1H), 8.05 (s, 1H), 7.66 (s, 1H), 7.50-7.56 (m, 1H), 7.33-7.42 (m, 4H), 7.25-7.32 (m, 2H), 5.15 (quin, J=7.4 Hz, 1H), 4.76 (d, J=4.4 Hz, 1H), 4.24 (td, J=6.8, 3.6 Hz, 1H), 3.92 (d, J=1.6 Hz, 2H), 3.51 (s, 3H), 2.86-2.90 (m, 2H), 2.71-2.78 (m, 1H), 2.62 (td, J=8.4, 5.6 Hz, 1H), 2.51-2.56 (m, 2H), 2.07 (s, 2H), 1.95-2.05 (m, 1H), 1.60 (qd, J=8.2, 5.3 Hz, 1H), 1.45 (d, J=7.2 Hz, 3H).

### General Procedure for Preparation of Compound 11

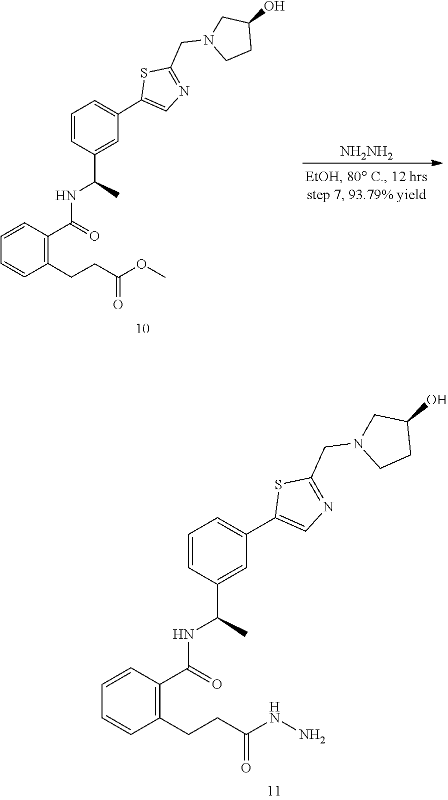 [SMILES: COC(=O)CCc1ccccc1C(=O)N[C@H](C)c1cccc(-c2cnc(CN3CC[C@H](O)C3)s2)c1 ; "NH2NH2" ; C[C@@H](NC(=O)c1ccccc1CCC(=O)NN)c1cccc(-c2cnc(CN3CC[C@H](O)C3)s2)c1]

To a solution of compound 10 (0.2 g, 405.17 μmol, 1 eq) in EtOH (2 mL) was added NH₂NH₂·H₂O (304.25 mg, 6.08 mmol, 295.38 μL, 15 eq). The mixture was stirred at 80° C. for 12 hrs. After cooling to room temperature, the reaction mixture was concentrated to give a residue. The residue was diluted with H₂O (10 mL) and the aqueous phase was extracted with EtOAc (10 mL×3). The combined organic layers were dried over Na₂SO₄, filtered and the filtrate was concentrated to give compound 11 (0.2 g, 380.01 μmol, 93.79% yield, 93.79% purity) as a white oil which was confirmed by ¹HNMR; LCMS.

LCMS: Rt=0.390 min, [M+1]=494.2, 93.79% purity.

¹H NMR (400 MHz, DMSO-d6) δ 8.83-8.93 (m, 2H), 8.05 (s, 1H), 7.64-7.68 (m, 1H), 7.53 (br d, J=6.8 Hz, 1H), 7.30-7.41 (m, 4H), 7.27 (s, 2H), 5.08-5.21 (m, 1H), 4.77 (d, J=4.4 Hz, 1H), 4.21-4.28 (m, 1H), 4.09-4.17 (m, 2H), 3.92 (d, J=1.4 Hz, 2H), 2.85-2.93 (m, 3H), 2.75 (q, J=7.8 Hz, 1H), 2.59-2.67 (m, 1H), 2.47 (br d, J=3.4 Hz, 1H), 2.33 (t, J=7.8 Hz, 2H), 1.99 (s, 1H), 1.56-1.65 (m, 1H), 1.46 (d, J=7.2 Hz, 3H).

### General Procedure for Preparation of NEU-7770

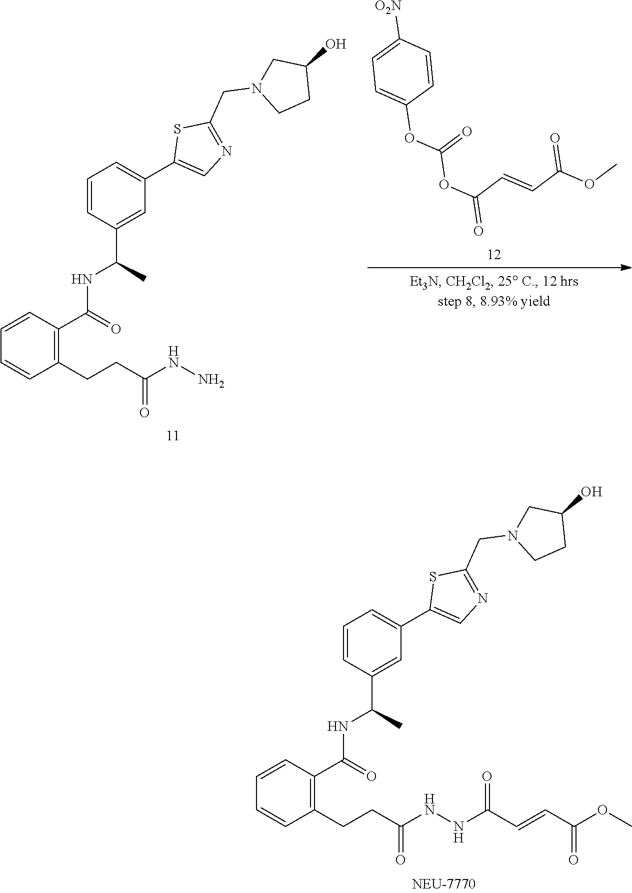

To a solution of compound 11 (0.2 g, 405.17 μmol, 1 eq) in CH₂Cl₂ (2 mL) were added Et₃N (49.20 mg, 486.20 μmol, 67.67 μL, 1.2 eq) and 01-methyl 04-(4-nitrophenoxy)carbonyl (E)-but-2-enedioate (179.41 mg, 607.76 μmol, 1.5 eq). The mixture was stirred at 25° C. for 12 hrs. The reaction mixture was concentrated to give a residue. The residue was purified by prep-HPLC (FA condition; column: Phenomenex Luna C18 75×30 mm×3 μm; mobile phase: [water(FA)-ACN]; B %: 10%-45%, 8 min) to give NEU-7770 (22.4 mg, 36.19 μmol, 8.93% yield, 97.85% purity) as a white solid which was confirmed by ¹HNMR; LCMS.

LCMS: Rt=1.821 min, [M+1]=606.2, 97.85% purity

¹H NMR (400 MHz, DMSO-d6) δ 10.55 (br s, 1H), 10.18 (s, 1H), 8.84 (br d, J=7.8 Hz, 1H), 8.06 (s, 1H), 7.66 (s, 1H), 7.53 (br d, J=7.2 Hz, 1H), 7.25-7.42 (m, 6H), 7.06 (d, J=15.4 Hz, 1H), 6.64-6.65 (m, 1H), 6.67 (d, J=15.4 Hz, 1H), 5.16 (br t, J=7.2 Hz, 1H), 4.77 (d, J=4.4 Hz, 1H), 4.24 (dt, J=6.4, 3.1 Hz, 1H), 3.92 (s, 2H), 3.70-3.76 (m, 3H), 2.84-2.94 (m, 3H), 2.72-2.78 (m, 1H), 2.55-2.69 (m, 2H), 2.44-2.49 (m, 2H), 2.02 (br dd, J=12.8, 7.4 Hz, 1H), 1.56-1.66 (m, 1H), 1.46 (br d, J=6.8 Hz, 3H).

### General Schematic for Synthesis of Compound 4.2 (NEU-7772)

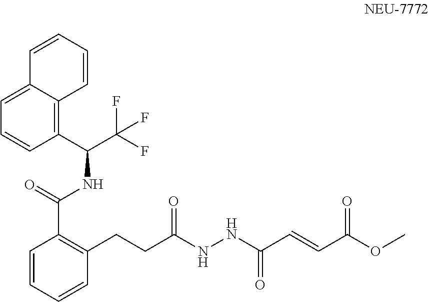 [SMILES: COC(=O)/C=C/C(=O)NNC(=O)CCc1ccccc1C(=O)N[C@@H](c1cccc2ccccc12)C(F)(F)F]

### General Schematic for Synthesis of Compound 4.2 (NEU-7772)

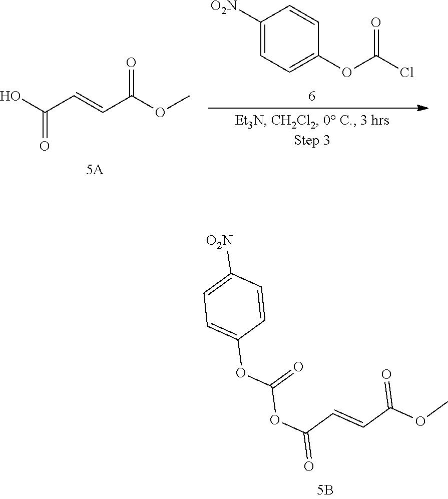

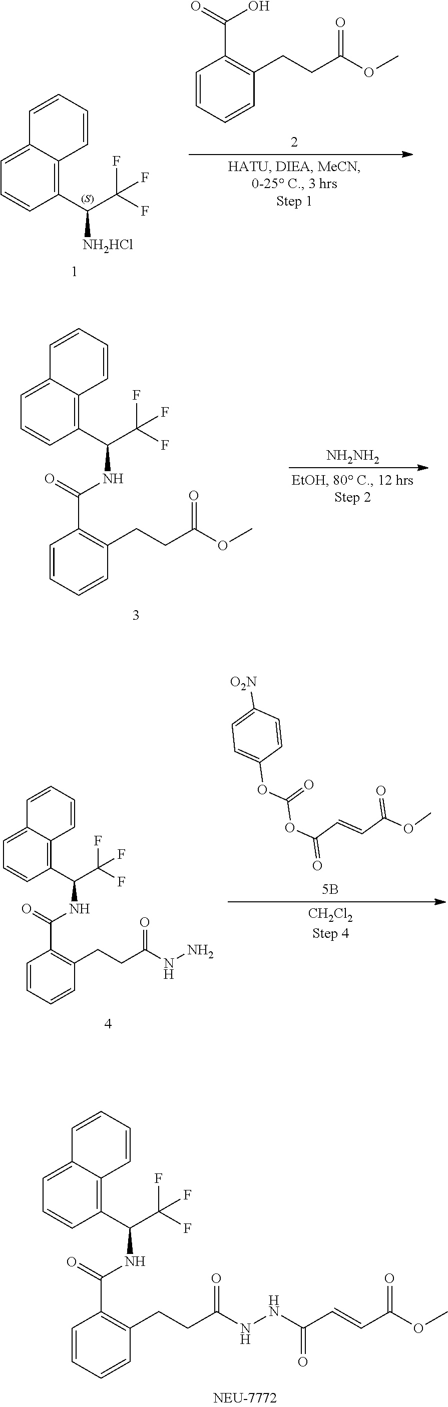

### General Procedure for Preparation of Compound 3

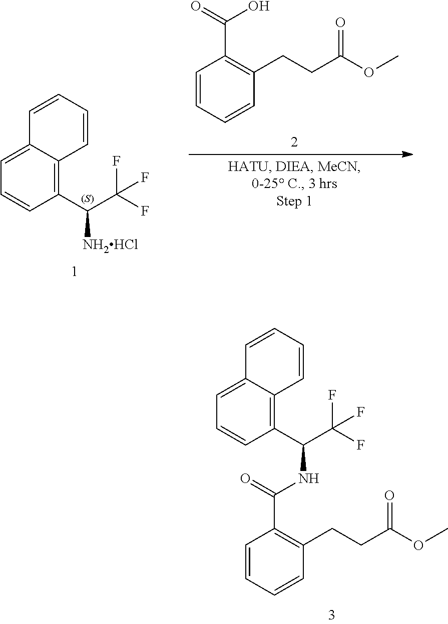

To a solution of compound 2 (83.81 mg, 402.51 μmol, 1.2 eq) in MeCN (1 mL) was added HATU (191.31 mg, 503.13 μmol, 1.5 eq) at 0° C. for 5 min. Then compound 1 (88.77 mg, 335.42 μmol, 1 eq, HCl) and DIEA (130.05 mg, 1.01 mmol, 175.27 μL, 3 eq) in MeCN (1 mL) was added to the mixture. The mixture was stirred at 25° C. for 3 hrs. The residue was diluted with H₂O (10 mL) and the aqueous phase was extracted with EtOAc (10 mL×3). The combined organic layers were dried over Na₂SO₄, filtered and the filtrate was concentrated to give a residue. The residue was purified by flash silica gel chromatography (ISCO®; 12 g SepaFlash® Silica Flash Column, eluent of 18˜20% ethyl acetate/petroleum ether gradient @ 60 mL/min) to give compound 3 (140 mg, crude) as white solid which was confirmed by 1H NMR.

¹H NMR: (400 MHz, DMSO-d₆) δ 9.89 (d, J=9.3 Hz, 1H), 8.29 (d, J=8.5 Hz, 1H), 8.03 (d, J=8.0 Hz, 2H), 7.92 (d, J=7.1 Hz, 1H), 7.71-7.65 (m, 1H), 7.64-7.57 (m, 2H), 7.43-7.37 (m, 1H), 7.31 (d, J=7.5 Hz, 1H), 7.29-7.26 (m, 2H), 6.91-6.80 (m, 1H), 3.53 (s, 3H), 2.92-2.85 (m, 2H), 2.58-2.52 (m, 2H).

### General Procedure for Preparation of Compound 4

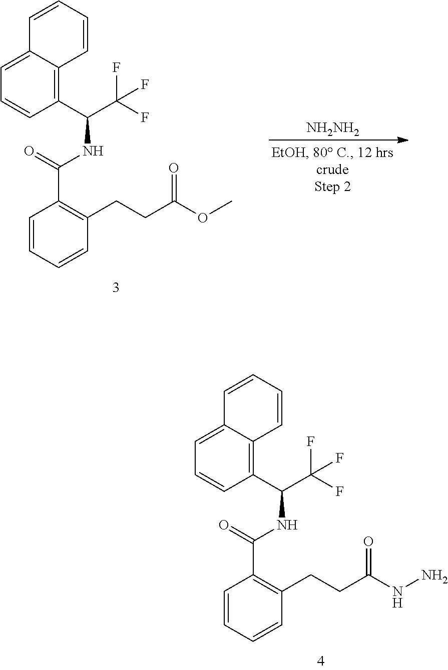 [SMILES: COC(=O)CCc1ccccc1C(=O)N[C@@H](c1cccc2ccccc12)C(F)(F)F ; "NH2NH2" ; NNC(=O)CCc1ccccc1C(=O)N[C@@H](c1cccc2ccccc12)C(F)(F)F]

To a solution of compound 3 (120 mg, 288.88 μmol, 1 eq) in EtOH (1.5 mL) was added NH₂NH₂·H₂O (216.92 mg, 4.33 mmol, 210.60 μL, 15 eq). The mixture was stirred at 80° C. for 12 hrs. After cooling to room temperature, the reaction mixture was concentrated to give a residue. The residue was diluted with H₂O (10 mL) and extracted with EtOAc (10 mL×3). The combined organic layers were dried over Na₂SO₄, filtered and the filtrate was concentrated to give compound 4 (120 mg, crude) as white solid which was confirmed by ¹H NMR.

¹H NMR: 400 MHz, DMSO-d6) δ=9.97 (d, J=9.2 Hz, 1H), 8.92 (br s, 1H), 8.29 (d, J=8.4 Hz, 1H), 8.03 (d, J=8.0 Hz, 2H), 7.94 (d, J=7.2 Hz, 1H), 7.73-7.65 (m, 1H), 7.64-7.58 (m, 2H), 7.42-7.36 (m, 1H), 7.28-7.25 (m, 2H), 6.90-6.80 (m, 1H), 4.14 (br s, 2H), 2.92-2.79 (m, 2H), 2.35 (t, J=7.6 Hz, 2H)

### General Procedure for Preparation of Compound 5

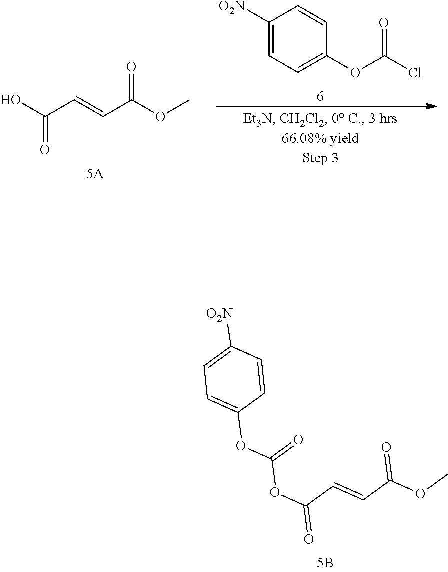

To a solution of compound 5A (1 g, 7.69 mmol, 1 eq) in CH₂Cl₂ (15 mL) was added Et₃N (933.77 mg, 9.23 mmol, 1.28 mL, 1.2 eq) and compound 6 (1.71 g, 8.46 mmol, 1.1 eq). The mixture was stirred at 0° C. for 3 hrs. After cooling to room temperature, the reaction mixture was concentrated to give compound 5B (1.5 g, 5.08 mmol, 66.08% yield) as a gray solid.

### General Procedure for Preparation of Compound NEU-7772

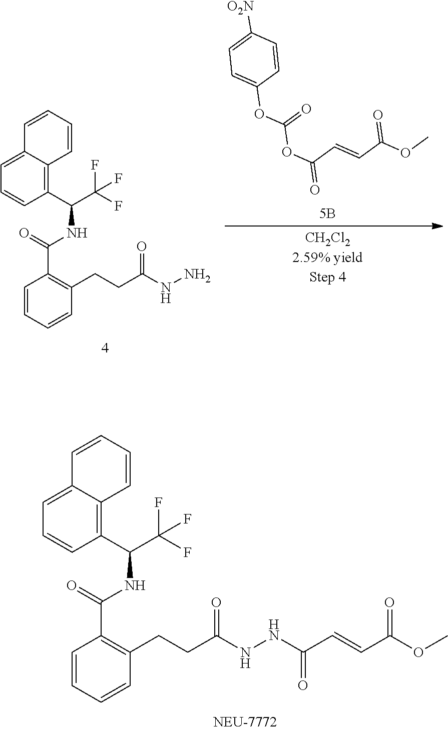 [SMILES: NNC(=O)CCc1ccccc1C(=O)N[C@@H](c1cccc2ccccc12)C(F)(F)F ; COC(=O)/C=C/C(=O)OC(=O)Oc1ccc([N+](=O)[O-])cc1 ; COC(=O)/C=C/C(=O)NNC(=O)CCc1ccccc1C(=O)N[C@@H](c1cccc2ccccc12)C(F)(F)F]

To a solution of Compound 5B (74.62 mg, 252.76 μmol, 1.5 eq) in CH₂Cl₂ (1 mL) was added compound 4 (70 mg, 168.51 μmol, 1 eq) at 0° C. The mixture was stirred at 20° C. for 2 hrs. After cooling to room temperature, the reaction mixture was concentrated to give a residue. The residue was purified by prep-HPLC (FA condition; column: Phenomenex Luna C18 75×30 mm×3 m; mobile phase: [water(FA)-ACN]; B %: 30%-70%, 8 min) to give NEU-7772 (2.4 mg, 4.37 μmol, 2.59% yield, 96% purity) as a white solid which was confirmed by LCMS and ¹H NMR.

LCMS, Rt=2.575, M+1=528.1, 96.79% purity.

¹H NMR (400 MHz, DMSO-d6) δ 10.55 (d, J=2.4 Hz, 1H), 10.18 (d, J=2.4 Hz, 1H), 9.89 (d, J=9.2 Hz, 1H), 8.29 (d, J=8.4 Hz, 1H), 8.02 (d, J=8.0 Hz, 2H), 7.92 (d, J=7.2 Hz, 1H), 7.71-7.67 (m, 1H), 7.61 (t, J=7.6 Hz, 2H), 7.42-7.38 (m, 1H), 7.36-7.33 (m, 1H), 7.27 (d, J=3.6 Hz, 2H), 7.07 (d, J=15.6 Hz, 1H), 6.86 (br t, J=8.3 Hz, 1H), 6.68 (d, J=15.6 Hz, 1H), 3.74 (s, 3H), 2.91-2.86 (m, 2H), 2.53-2.52 (m, 2H).

### Synthesis of Compound 5.2 (NEU-7792)

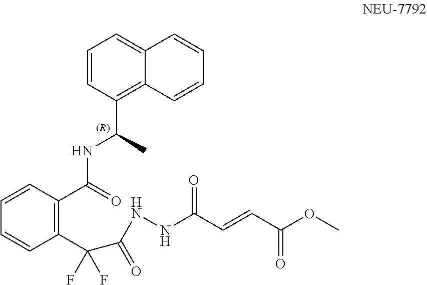

### General Schematic for Synthesis of Compound 5.2 (NEU-7792)

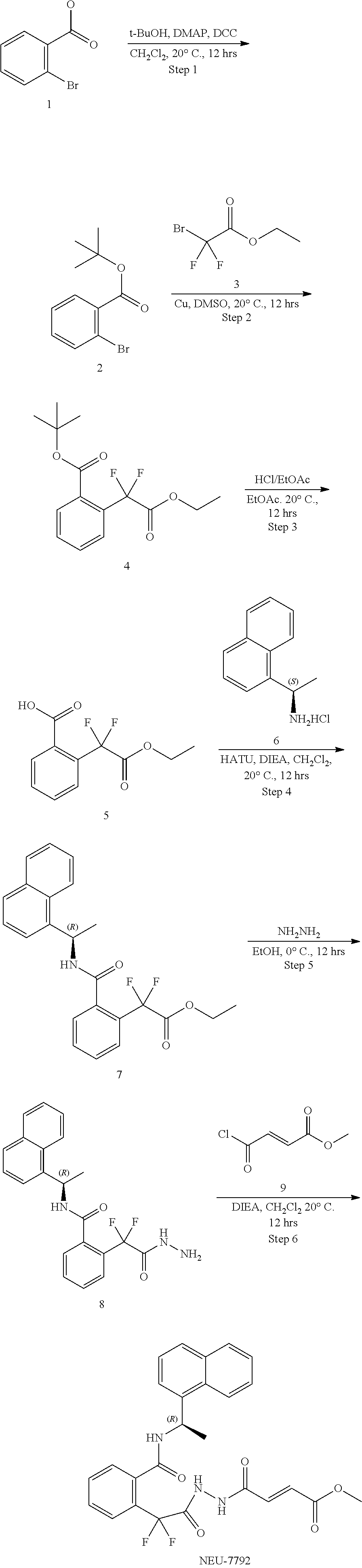

### Procedure for Preparation of Compound 2

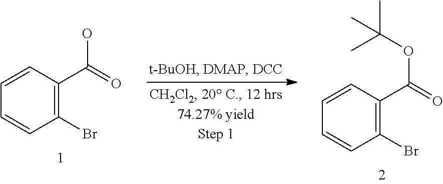

To a solution of compound 1 (10 g, 49.75 mmol, 1 eq) in CH₂Cl₂ (100 mL) were added t-BuOH (5.53 g, 74.62 mmol, 7.14 mL, 1.5 eq), DMAP (607.75 mg, 4.97 mmol, 0.1 eq) and DCC (10.26 g, 49.75 mmol, 10.06 mL, 1 eq). The mixture was stirred at 20° C. for 12 hrs. The resultant mixture was filtered and the filter cake was rinsed with EtOAc (50 mL×3). Then the combined filtrates were diluted with H₂O (50 mL) and extracted with EtOAc (50 mL×3). The combined organic layers were dried over Na₂SO₄, filtered and the filtrate was concentrated to give a residue. The residue was purified by flash silica gel chromatography (ISCO®; 120 g SepaFlash® Silica Flash Column, Eluent of 10˜15% Ethyl acetate/Petroleum ether gradient @ 100 mL/min) to give compound 2 (9.5 g, 36.95 mmol, 74.27% yield) as yellow oil which was confirmed by ¹H NMR.

¹H NMR: (400 MHz, DMSO-d6) δ 7.68 (dd, J=1.6, 7.8 Hz, 1H), 7.64 (dd, J=2.0, 7.4 Hz, 1H), 7.47-7.39 (m, 2H), 1.54 (s, 9H)

### Procedure for Preparation of Compound 4

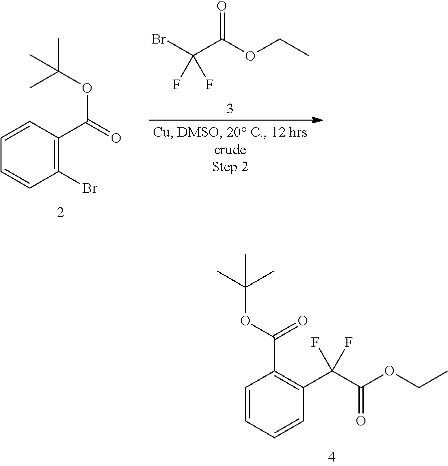

To a suspension of Cu (8.90 g, 140.01 mmol, 992.98 μL, 4 eq) in DMSO (90 mL) was added ethyl 2-bromo-2,2-difluoro-acetate (14.21 g, 70.01 mmol, 9.02 mL, 2 eq) at 20° C., and the mixture was kept stirring for 1 hr at 20° C. under N₂ atmosphere. Then compound 2 (9 g, 35.00 mmol, 1 eq) was added, and the mixture was stirred at 20° C. for 11 hrs. The reaction mixture was concentrated to give a residue. The residue was diluted with H₂O (50 mL) and extracted with EtOAc (50 mL×3). The organic phase is washed with brine and the combined organic layers were dried over Na₂SO₄, filtered and the filtrate was concentrated to give a residue. The residue was purified by flash silica gel chromatography (ISCO®; 220 g SepaFlash® Silica Flash Column, Eluent of 20-30% Ethyl acetate/Petroleum ether gradient @ 120 mL/min) to give compound 4 (13.5 g, crude) as yellow oil which was confirmed by ¹H NMR.

¹H NMR: (400 MHz, DMSO-d6) δ 7.90 (d, J=7.6 Hz, 1H), 7.84-7.78 (m, 1H), 7.75 (dt, J=1.2, 7.5 Hz, 1H), 7.71-7.66 (m, 1H), 4.26 (q, J=7.2 Hz, 2H), 1.50 (s, 9H), 1.20 (t, J=7.2 Hz, 3H)

### Procedure for Preparation of Compound 5

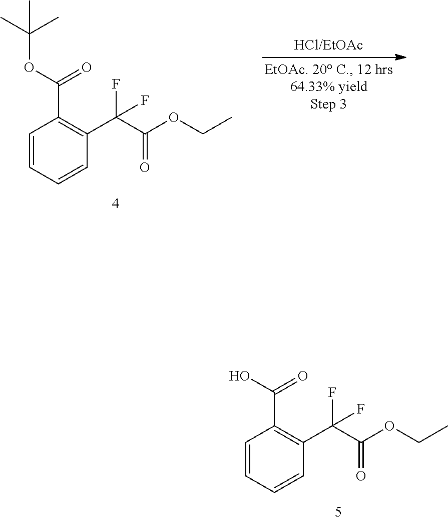 [SMILES: CCOC(=O)C(F)(F)c1ccccc1C(=O)OC(C)(C)C ; CCOC(=O)C(F)(F)c1ccccc1C(=O)O]

To a solution of compound 4 (13 g, 43.29 mmol, 1 eq) in EtOAc (70 mL) was added HCl/EtOAc (4 M, 70 mL, 6.47 eq). The mixture was stirred at 20° C. for 12 hrs under N₂ atmosphere. The reaction mixture was concentrated to give a residue. The residue was diluted with H₂O (20 mL) and extracted with EtOAc (20 mL×3). The combined organic layers were dried over Na₂SO₄, filtered and the filtrate was concentrated to give a residue. The residue was purified by flash silica gel chromatography (ISCO®; 120 g SepaFlash® Silica Flash Column, Eluent of 20˜30% Ethyl acetate/Petroleum ether gradient @ 80 mL/min) to give compound 5 (6.8 g, 27.85 mmol, 64.33% yield) as white solid which was confirmed by LCMS and ¹H NMR.

LCMS R t=0.505 min, [M+1]=243.0, 98.8% purity ¹H NMR (400 MHz, DMSO-d6) 8.01 (d, J=7.6 Hz, 1H), 7.87-7.82 (m, 1H), 7.77 (dt, J=1.2, 7.6 Hz, 1H), 7.73-7.68 (m, 1H), 4.24 (q, J=7.2 Hz, 2H), 1.20 (t, J=7.2 Hz, 3H)

### Procedure for Preparation of Compound 7

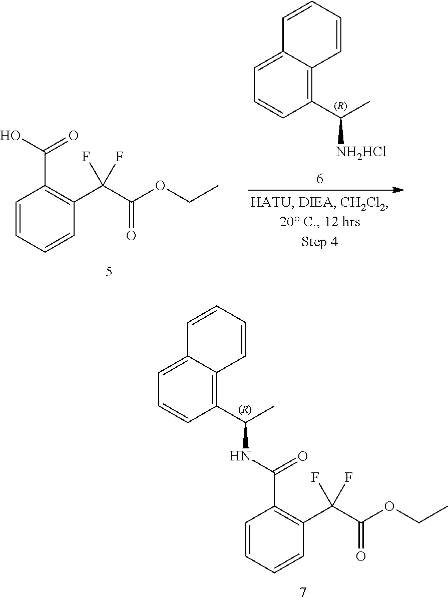

To a solution of compound 5 (2 g, 8.19 mmol, 1 eq) in CH₂Cl₂ (30 mL) was added DIEA (3.18 g, 24.57 mmol, 4.28 mL, 3 eq), 6 (2.04 g, 9.83 mmol, 1.2 eq) and HATU (4.67 g, 12.29 mmol, 1.5 eq). The mixture was stirred at 20° C. for 12 hrs. The reaction mixture was concentrated to give a residue. The residue was diluted with H₂O (20 mL) and extracted with EtOAc (20 mL×3). The combined organic layers were dried over Na₂SO₄, filtered and the filtrate was concentrated to give a residue. The residue was purified by flash silica gel chromatography (ISCO®; 80 g SepaFlash® Silica Flash Column, Eluent of 5˜10% Ethyl acetate/Petroleum ether gradient @ 70 mL/min) to give compound 7 (3.2 g, 8.05 mmol, 98.31% yield) as brown solid which was confirmed by LCMS and 1H NMR.

LCMS R t=0.577 min, [M+1]=398.1, 90% purity

1H NMR: (400 MHz, DMSO-d6) δ 9.21 (d, J=8.0 Hz, 1H), 8.19 (d, J=8.3 Hz, 1H), 7.95 (d, J=8.0 Hz, 1H), 7.85 (d, J=8.0 Hz, 1H), 7.78-7.73 (m, 1H), 7.73-7.68 (m, 1H), 7.68-7.63 (m, 3H), 7.60-7.51 (m, 3H), 5.86 (quin, J=7.2 Hz, 1H), 4.17 (q, J=7.2 Hz, 2H), 1.61 (d, J=7.2 Hz, 3H), 1.21-1.15 (m, 3H)

### Procedure for Preparation of Compound 8

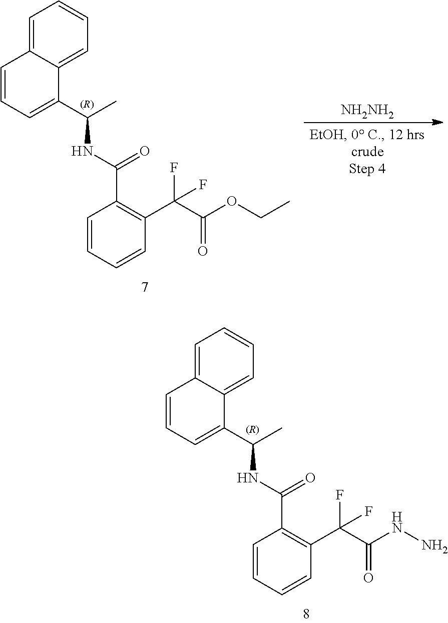

To a solution of compound 7 (0.5 g, 1.26 mmol, 1 eq) in EtOH (10 mL) was added NH₂NH₂·H₂O (1.89 g, 37.74 mmol, 1.83 mL, 30 eq). The mixture was stirred at 0° C. for 12 hrs. The reaction mixture was concentrated to give a residue. The residue was diluted with H₂O (10 mL) and extracted with EtOAc (10 mL×3). The combined organic layers were dried over Na₂SO₄, filtered and the filtrate was concentrated to give a residue to give compound 8 (550 mg, crude) as yellow solid which was confirmed by ¹H NMR.

¹H NMR: (400 MHz, DMSO-d6) δ 9.26 (br d, J=7.6 Hz, 1H), 8.93 (br s, 1H), 8.19 (d, J=8.4 Hz, 1H), 7.96 (d, J=8.0 Hz, 1H), 7.85 (d, J=8.0 Hz, 1H), 7.67-7.57 (m, 5H), 7.55-7.46 (m, 3H), 5.90 (quin, J=7.2 Hz, 1H), 4.14 (br s, 2H), 1.59 (d, J=7.0 Hz, 3H)

### Procedure for Preparation of Compound NEU-7792

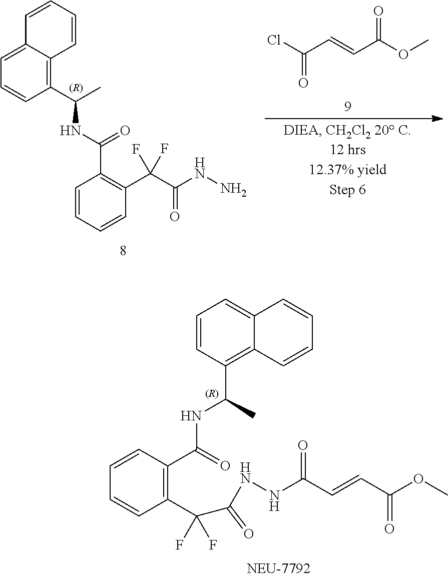

To a solution of compound 8 in CH₂Cl₂ (8 mL) was added DIEA (202.26 mg, 1.56 mmol, 272.59 μL, 3 eq) and methyl (E)-4-chloro-4-oxo-but-2-enoate (154.98 mg, 1.04 mmol, 2 eq). The mixture was stirred at 20° C. for 12 hrs. The reaction mixture was concentrated to give a residue. The residue was diluted with H₂O (10 mL) and extracted with EtOAc (10 mL×3). The combined organic layers were dried over Na₂SO₄, filtered and the filtrate was concentrated to give a residue. The residue was purified by prep-HPLC (neutral condition; column: Phenomenex Luna C18 100×30 mm×3 um; mobile phase: [water(0.2% FA)-ACN]; gradient: 35%-55% B over 8.0 min) to give NEU-7792 (32.1 mg, 64.53 μmol, 12.37% yield, 99.6% purity) as brown solid which was confirmed by LCMS and ¹H NMR.

LCMS R t=2.602 min, [M+1]=496.2, 99.6% purity

¹H NMR (400 MHz, DMSO-d6) δ 11.10 (s, 1H), 10.77 (s, 1H), 9.24 (d, J=8.0 Hz, 1H), 8.18 (d, J=8.4 Hz, 1H), 7.96 (d, J=7.6 Hz, 1H), 7.85 (d, J=8.0 Hz, 1H), 7.69-7.66 (m, 1H), 7.66-7.61 (m, 3H), 7.60 (dd, J=1.2, 8.4 Hz, 1H), 7.54 (br d, J=8.4 Hz, 3H), 7.01 (d, J=15.2 Hz, 1H), 6.69 (d, J=15.6 Hz, 1H), 5.93-5.87 (m, 1H), 3.73 (s, 3H), 1.59 (d, J=6.8 Hz, 3H)

### Synthesis of Compound 6.2 (NEU-8114)

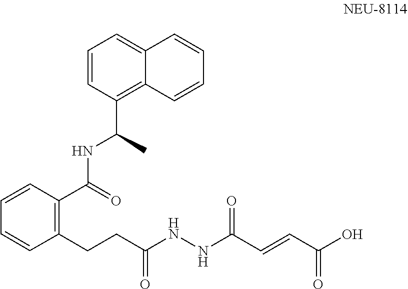 [SMILES: C[C@@H](NC(=O)c1ccccc1CCC(=O)NNC(=O)/C=C/C(=O)O)c1cccc2ccccc12]

To a solution of methyl (R,E)-4-(2-(3-(2-((1-(naphthalen-1-yl)ethyl)carbamoyl)phenyl)propanoyl)hydrazineyl)-4-oxobut-2-enoate in THF (0.1 M) a 1M LiOH solution was added. The reaction mixture was stirred at 25° C. for 10 h. It was then concentrated under reduced pressure and 1M HCl was added. The obtained precipitate was washed with hexanes, Et₂O and cold DCM, affording the desired carboxylic acids in pure form in quantitative yield.

¹H NMR (400 MHz, DMSO-d6)¹H NMR (500 MHz, DMSO) δ 10.47 (s, 1H), 10.12 (s, 1H), 8.93 (d, J=7.8 Hz, 1H), 8.24 (d, J=8.6 Hz, 1H), 7.95 (d, J=8.1 Hz, 1H), 7.83 (d, J=8.2 Hz, 1H), 7.66-7.48 (m, 4H), 7.33 (dd, J=18.5, 6.8 Hz, 3H), 7.26 (d, J=7.1 Hz, 1H), 6.99 (dd, J=15.7, 3.3 Hz, 1H), 6.68-6.58 (m, 1H), 5.93 (t, J=7.5 Hz, 1H), 2.93 (d, J=8.0 Hz, 2H), 2.54-2.48 (m, 3H), 1.62-1.56 (m, 3H), 1.24 (s, 1H), 0.85 (s, 1H).

### Synthesis of Compound 7.2

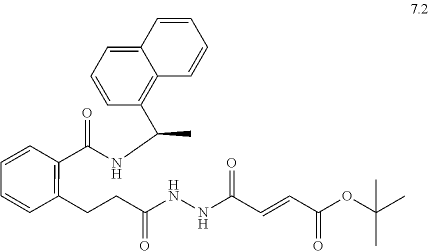 [SMILES: C[C@@H](NC(=O)c1ccccc1CCC(=O)NNC(=O)/C=C/C(=O)OC(C)(C)C)c1cccc2ccccc12]

To a solution of Compound 6.2 in DMF was added tert-butyl 2,2,2-trichloroacetimidate at 0° C. and allowed to warm to 25° C. and stirred for 18 h. The solution was extracted with EtOAc and saturated sodium bicarbonate solution to afford crude Compound 7.2.

¹H NMR (400 MHz, DMSO) δ 10.56 (dd, J=13.6, 2.7 Hz, 1H), 10.21 (d, J=2.7 Hz, 1H), 8.98 (d, J=7.9 Hz, 1H), 8.24 (d, J=8.5 Hz, 1H), 8.00-7.93 (m, 1H), 7.84 (d, J=8.1 Hz, 1H), 7.76-7.48 (m, 5H), 7.40-7.22 (m, 7H), 7.08 (d, J=15.5 Hz, 1H), 6.69 (d, J=15.6 Hz, 1H), 5.97-5.89 (m, 1H), 3.75 (s, 2H), 2.94 (t, J=7.9 Hz, 2H), 2.39 (d, J=9.8 Hz, 1H), 2.22 (dt, J=31.5, 7.9 Hz, 2H), 1.67 (s, 2H), 1.59 (d, J=6.9 Hz, 3H), 1.48 (s, 2H), 1.24 (s, 9H), 1.17 (t, J=7.1 Hz, 4H).

The compounds containing ester substituents or succinyl methyl ester (7.2, 8.2, 9.2, 10.2) were prepared through coupling of the corresponding acyl chloride of any given succinyl or fumaryl ester by the procedure analogous to that used in Sanders et al. Nature Communications 14, 1733 (2023). In some cases, the acyl chloride is prepared in situ by reaction with 1.2 equivalents of oxalyl chloride with the corresponding carboxylic acid in dichloromethane and used immediately in solution without purification. Briefly, a solution of the (R)-2-(3-hydrazineyl-3-oxopropyl)-N-(1-(naphthalen-1-yl)ethyl)benzamide was stirred in minimal volume of dichloromethane at RT until fully dissolved. The acyl chloride derivative was added dropwise over 5 minutes. Precipitation of the desired product occurs within 1 hour and is filtered and washed with ether and dried under vacuum.

Compound 7.2 ¹H NMR (400 MHz, DMSO) δ 10.56 (s, 1H), 10.21 (s, 1H), 8.98 (d, J=7.9 Hz, 1H), 8.24 (d, J=8.5 Hz, 1H), 8.02-7.90 (m, 1H), 7.84 (d, J=8.1 Hz, 1H), 7.76-7.46 (m, 5H), 7.45-7.21 (m, 6H), 7.08 (d, J=15.5 Hz, 1H), 6.69 (d, J=15.6 Hz, 1H), 6.00-5.85 (m, 1H), 2.94 (t, J=7.9 Hz, 2H), 2.53 (br, 2H), 1.59 (d, J=6.9 Hz, 3H), 1.24 (s, 9H).

Compound 8.2 ¹H NMR (400 MHz, DMSO) δ 9.79 (dd, J=13.7, 1.9 Hz, 2H), 9.10 (br, 2H), 8.99 (s, 1H), 8.24 (d, J=8.5 Hz, 1H), 7.96 (dd, J=8.2, 1.5 Hz, 1H), 7.84 (d, J=8.1 Hz, 1H), 7.63 (t, J=6.7 Hz, 2H), 7.58-7.48 (m, 2H), 7.41-7.20 (m, 4H), 5.92 (p, 1H), 2.92 (m, 2H), 2.57-2.37 (m, 6H), 1.58 (d, J=6.9 Hz, 3H).

Compound 9.2 ¹H NMR (400 MHz, DMSO) δ 10.60 (d, J=2.4 Hz, 1H), 10.22 (d, J=2.5 Hz, 1H), 8.99 (d, J=7.9 Hz, 1H), 8.24 (d, J=8.4 Hz, 1H), 7.96 (dd, J=8.2, 1.5 Hz, 1H), 7.84 (d, J=8.1 Hz, 1H), 7.66-7.58 (m, 2H), 7.58-7.48 (m, 2H), 7.39-7.22 (m, 4H), 7.09 (d, J=15.8 Hz, 1H), 6.68 (dd, J=15.6, 7.1 Hz, 1H), 5.93 (p, J=7.0 Hz, 1H), 4.21 (q, J=7.1 Hz, 3H), 2.93 (t, J=7.4, 4.5 Hz, 2H), 2.53 (s, 2H), 1.59 (d, J=6.9 Hz, 3H), 1.23 (m, 2H).

Compound 10.2 ¹H NMR (500 MHz, DMSO) δ 10.58 (s, 1H), 10.21 (s, 1H), 8.95 (d, J=7.9 Hz, 1H), 8.22 (d, J=8.6 Hz, 1H), 7.94 (d, J=8.1 Hz, 1H), 7.82 (d, J=8.1 Hz, 1H), 7.65-7.56 (m, 2H), 7.56-7.47 (m, 2H), 7.44-7.27 (m, 8H), 7.24 (t, J=7.5 Hz, 1H), 7.12 (dd, J=15.6, 2.3 Hz, 1H), 6.72 (dd, J=15.5, 2.4 Hz, 1H), 5.91 (p, J=7.5 Hz, 1H), 5.23 (d, J=2.3 Hz, 2H), 2.93 (dd, J=13.8, 5.2 Hz, 2H), 2.54-2.47 (m, 2H), 1.58 (d, J=6.9 Hz, 3H).

Compounds 11.2 and 12.2 were prepared in the same manner as Compound 7 based on Nature Communications 14, 1733 (2023). However, instead of 2-(3-methoxy-3-oxopropyl)benzoic acid, the 5-((tert-butoxycarbonyl)amino)-2-(3-methoxy-3-oxopropyl)benzoic acid was used in the initial amide coupling with (R)-(+)-1-(1-naphthyl)ethylamine. 12.2 was then prepared from 11.2 by deprotection of the t-butyloxycarbonyl (BOC) group by trifluoroacetic acid. Briefly, Compound 11.2 (500 mg; 0.85 mmol) was dissolved in 100 mL dichloromethane. Trisisopropylsilane (1.34 g; 1.75 mL; 10 eq) are added and solution is cooled to 0° C. in an ice bath. Trifluoroacetic acid (10 mL) are added slowly via syringe, the reactions is then allowed to warm to RT. After 30 min at RT the reaction is complete by MS/ESI. The solvents are removed on rotary evaporator at RT, residual trisisopropylsilane removed on HV to yield a viscous oil. The product is purified by preparative HPLC.

### NMR Data

Compound 11.2 ¹H NMR (300 MHz, DMSO) δ 10.60 (s, 1H), 10.23 (s, 1H), 9.42 (s, 1H), 8.28 (d, J=8.4 Hz, 2H), 7.99 (dd, J=8.1, 1.5 Hz, 2H), 7.87 (d, J=8.1 Hz, 2H), 7.71-7.50 (m, 7H), 7.46 (d, J=9.7 Hz, 3H), 7.22 (d, J=8.1 Hz, 2H), 7.12 (d, J=15.6 Hz, 2H), 6.73 (d, J=15.6 Hz, 1H), 5.95 (d, J=6.9 Hz, 2H), 3.78 (s, 4H), 3.38 (s, 12H), 2.88 (t, J=7.8 Hz, 3H), 2.49 (t, J=5.0 Hz, 3H), 1.61 (d, J=6.8 Hz, 3H), 1.50 (s, 9H).

Compound 12.2 ¹H NMR (300 MHz, DMSO) δ 10.22 (s, 2H), 9.85 (s, 2H), 8.66 (d, J=7.9 Hz, 2H), 7.87 (d, J=8.4 Hz, 2H), 7.59 (d, J=7.9 Hz, 2H), 7.48 (d, J=8.1 Hz, 2H), 7.21 (dt, J=21.6, 7.7 Hz, 9H), 6.86 (d, J=8.2 Hz, 2H), 6.72 (d, J=15.6 Hz, 2H), 6.68-6.56 (m, 5H), 6.32 (d, J=15.6 Hz, 2H), 5.56 (p, J=7.0 Hz, 3H), 4.26 (s, 1H), 3.38 (s, 5H), 2.50 (t, J=6.8 Hz, 4H), 2.17-2.06 (m, 5H), 1.21 (d, J=6.8 Hz, 6H).

SARS-CoV-2 antiviral assays. Initial screening to measure cytopathic effect (CPE) protection for the 50% efficacy concentration (EC₅₀) and cytotoxicity (CC₅₀) was performed using an assay based on African green monkey kidney epithelial (Vero E6) cells in 384-well plates. Each plate can evaluate five compounds in duplicate at seven concentrations to measure an EC₅₀ and CC₅₀. Each plate included three controls: cells alone (uninfected control), cells with SARS-CoV-2 (infected control) for plate normalization, and remdesivir as a drug control. Cell viability was measured using the CellTiter-Glo Luminescent Cell Viability Assay. In brief, Vero E6 TMPRSS ACE2 cells were grown to ˜90% confluency in 384-well plates and treated for 1 hr with compounds. Cells were infected at an MOI=0.1 of SARS-CoV-2 isolate USA-WA1/2020. After 48 h, the SARS-CoV-2-mediated CPE and cytotoxicity were assessed by measuring live cells using CellTiter-Glo. The selectivity index at 50% (SI₅₀) was then calculated from the EC₅₀ and CC₅₀ values. To ensure robust and reproducible signals, each 384-well plate was evaluated for its Z-score, signal to noise, signal to background, and coefficient of variation. This assay has been validated for use in high-throughput format for single-dose screening and is sensitive and robust, with Z values>0.5, signal to background>20, and signal to noise>3.3. Antiviral activity and cytotoxicity were also assessed with compound in the presence of 2 μM CP-100356 and SARS-CoV-2. Following incubation for 48 hours at 5% CO₂ and 37° C., the percent cell viability was measured with CellTiterGlo. Signals were read with a multimode plate reader. Cells alone (positive control) and cells plus virus (negative control) were set to 100% and 0% cell viability to normalize the data from the compound testing. Data were normalized to cells (100%) and virus (0%) plus cells. Each concentration was tested in duplicate.

Compounds were also tested against SARS-CoV-2 variants using Vero E6 cells using methods described previously (H. Liu et al., Nat. Commun. 13(1), 1891, 2022). Confluent or near-confluent cell culture monolayers of Vero E6 cells were prepared in 96-well disposable microplates the day before testing. Cells were maintained in Modified Eagle Medium (MEM) supplemented with 5% fetal bovine serum (FBS). For antiviral assays the same medium was used but with FBS reduced to 2% and supplemented with 50 μg/ml gentamicin. Compounds were dissolved in DMSO, saline, or the diluent requested by the submitter. Less soluble compounds were vortexed, heated, and sonicated, and, if they still did not go into solution, were tested as colloidal suspensions. Each test compound was prepared at four serial log 10 concentrations, usually 0.1, 1.0, 10, and 100 μg/ml or μM (per sponsor preference). Lower concentrations were used when insufficient compound was supplied. Five microwells were used per dilution: three for infected cultures and two for uninfected toxicity cultures. Controls for the experiment consisted of six microwells that were infected and not treated (virus controls) and six that were untreated and uninfected (cell controls) on every plate. A known active drug was tested in parallel as a positive control drug using the same method applied for test compounds. The positive control was tested with every test run.

TABLE 1
EC₅₀ data for Vero E6 cells

| | | CPE | CPE | Vero E6 |
|---|---|---|---|---|
| | | EC50 | EC90 | CC50 |
| ID | Structure | (μM) | (μM) | (μM) |
| NEU-8114 | 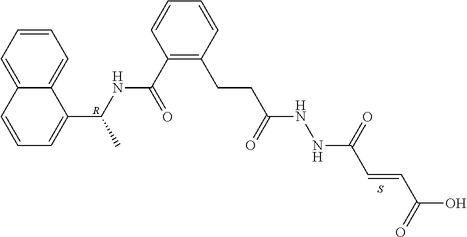 | 0.015 | 0.16 | >10.0 |
| NEU-7792 | 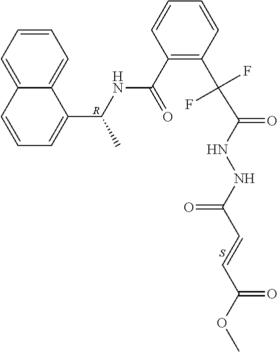 | 0.24 | 0.82 | >10.0 |
| NEU-7772 | 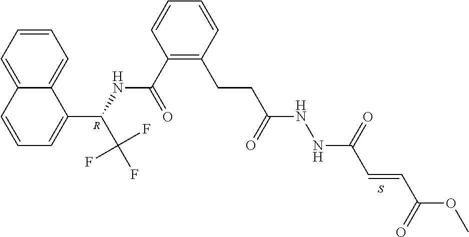 | >10.0 | | >10.0 |
| NEU-7770 | 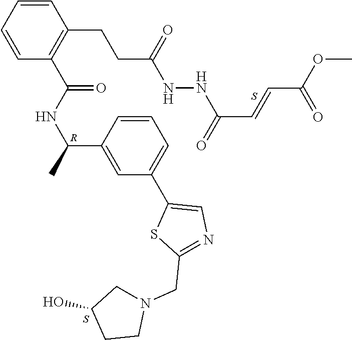 | 0.14 | 0.88 | >10.0 |
| NEU-7769 | 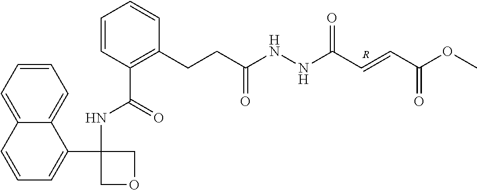 | 1.5 | 3.2 | >10.0 |
| NEU-7768 | 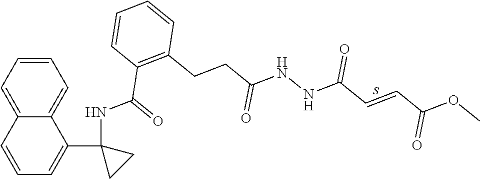 | 0.022 | 0.25 | >10.0 |

### Further Exemplary Compounds

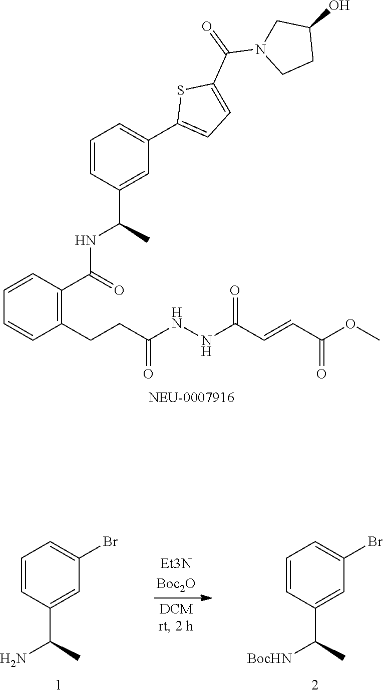

Synthetic Procedure: (R)-1-(3-bromophenyl)ethanamine (2.41 mL, 16.0 mmol) was dissolved in DCM (32.0 mL). Then, Et₃N (395 μL, 24.0 mmol) and Boc₂O (4.19 g, 19.2 mmol) were added. The mixture was stirred at 25° C. for 2 hours. Next, the mixture was concentrated to give a residue. The residue was diluted with H₂O (50 mL) and extracted with EtOAc (3×50 mL). The combined organic layers were dried over Na₂SO₄, filtered and the filtrate was concentrated to give a residue. Then, it was purified by flash column chromatography using hexane to Hexane:Ethyl acetate (9:1) to give compound 2 (4.75 g, 99%) as a white solid.

¹H NMR (500 MHz, CDCl₃, ppm) δ: 7.45 (s, 1H, Ar) 7.38 (d, J=7.5 Hz, 1H, Ar) 7.18-7.25 (m, 2H, Ar) 4.77 (br. s., 1H, NH) 1.35-1.50 (m, 12H, CH₃).

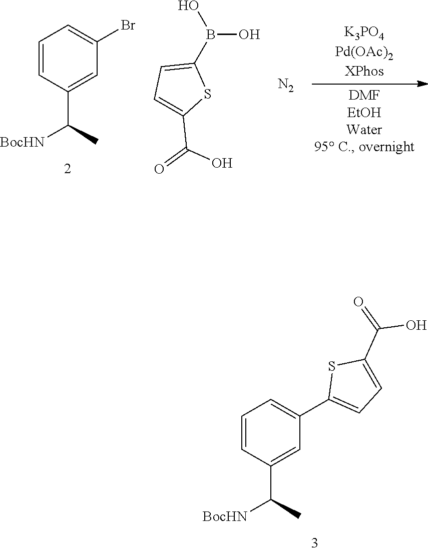 [SMILES: C[C@@H](NC(=O)OC(C)(C)C)c1cccc(Br)c1 ; O=C(O)c1ccc(B(O)O)s1 ; "N2" ; C[C@@H](NC(=O)OC(C)(C)C)c1cccc(-c2ccc(C(=O)O)s2)c1]

Synthetic Procedure: To a solution of compound 2 (500 mg, 1.67 mmol) in degassed DMF (1 mL), EtOH (1.00 mL) and water (0.500 mL), 2-carboxythiophene-5-boronic acid (430 mg, 2.50 mmol), XPhos (39.7 mg, 83.3 μmol), Pd(OAc)₂ (18.7 mg, 83.3 mol) and K₃PO₄ (884 mg, 4.16 mmol) were added. Then, the mixture was stirred at 95° C. overnight. After cooling to room temperature, the reaction mixture was diluted with ethyl acetate (50 mL), filtered through celite, and concentrated in vacuo to give a residue. The crude was purified by reverse phase flash column chromatography using water to water:methanol (1:1) as eluent to give compound 3 (510 mg, 88%) as a white solid.

¹H NMR (500 MHz, DMSO-d₆, ppm) δ: 7.54 (s, 1H, Ar) 7.45 (d, J=7.5 Hz, 2H, Ar+NH) 7.31 (t, J=7.6 Hz, 1H, Ar) 7.26 (d, J=3.7 Hz, 1H, Ar) 7.19 (d, J=7.6 Hz, 1H, Ar) 7.15 (d, J=3.7 Hz, 1H, Ar) 4.56-4.70 (m, 1H, CH) 1.37 (s, 9H, CH₃ [Boc]) 1.32 (d, J=7.0 Hz, 3H, CH₃ [CH—CH₃]).

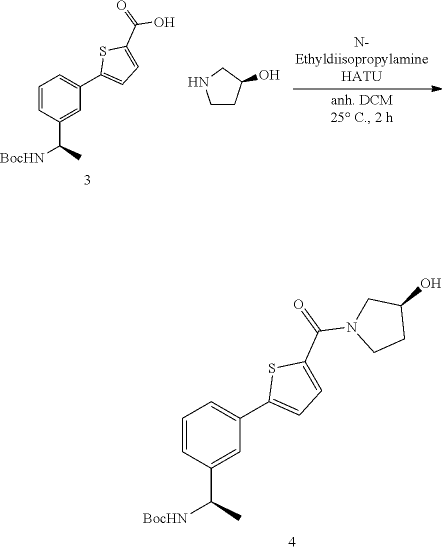 [SMILES: C[C@@H](NC(=O)OC(C)(C)C)c1cccc(-c2ccc(C(=O)O)s2)c1 ; O[C@H]1CCNC1 ; C[C@@H](NC(=O)OC(C)(C)C)c1cccc(-c2ccc(C(=O)N3CC[C@H](O)C3)s2)c1]

Synthetic Procedure: Compound 3 (50.0 mg, 144 μmol) and HATU (341 mg, 898 mol) were placed in an under N₂ atmosphere and anh. DCM (2.60 mL) was added. The mixture was stirred at rt for 1 hour. Then, 3-pyrrolidinol (104 μL, 898 μmol) and N-ethyldiisopropylamine (392 μL, 2.25 mmol) were added and the reaction mixture was stirred at 25° C. for 2 hours. The solvents were evaporated to give a residue that was redissolved in EtOAc (20 mL) and washed with water (2×15 mL) and brine (2×15 mL) to afford compound 4 (297 mg, 95%) as a yellowish solid.

¹H NMR (500 MHz, DMSO-d₆, ppm) δ: 7.64 (br. s., 1H, Ar), 7.58 (d, J=7.3 Hz, 2H, Ar), 7.51 (d, J=4.0 Hz, 1H, Ar), 7.46 (d, J=7.9 Hz, 1H, NH), 7.38 (t, J=7.7 Hz, 1H, Ar), 7.29 (d, J=7.6 Hz, 1H, Ar), 5.04 (d, J=18.5 Hz, 1H, OH), 4.70-4.60 (m, 1H, CH), 4.34 (d, J=36.6 Hz, 1H, CH), 3.94-3.83 (m, 1H, CH₂), 3.66-3.56 (m, 2H, CH₂), 3.55-3.42 (m, 1H, CH₂), 2.07-1.77 (m, 2H, CH₂), 1.37 (s, 9H, CH₃[Boc]), 1.33 (d, J=7.02 Hz, 3H, CH₃).

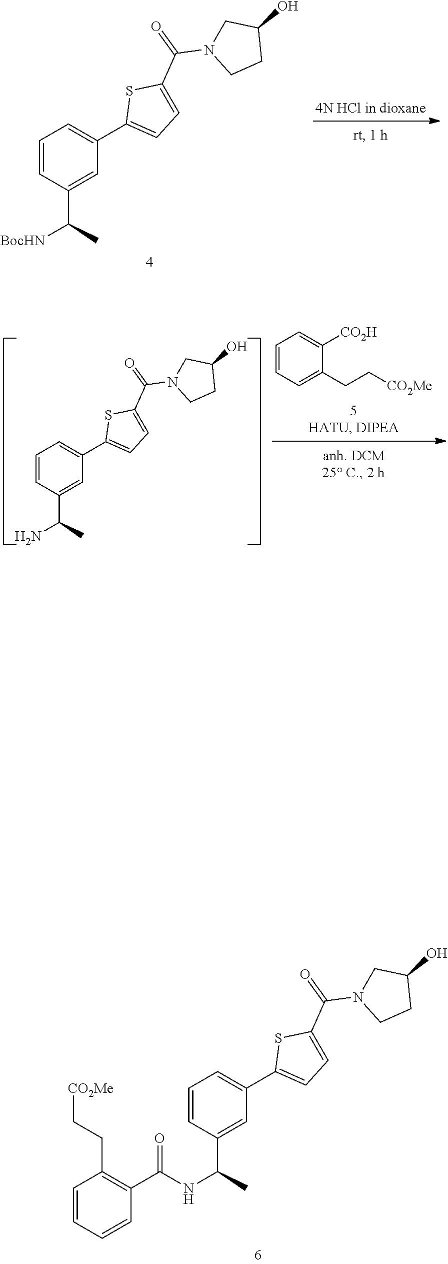

Synthetic Procedure: Compound 4 (289 mg, 694 μmol) was dissolved in 2 mL of dioxane. Then, a solution 4N HCl in dioxane (1.73 mL, 6.94 mmol) was added. After the addition, a pink precipitate was formed. The mixture was stirred for 1 hour. The solid was collected by filtration, rinsed with EtOAc (5 mL×3) and dried under reduced pressure to give the crude as a pink solid that was used in the next step without purification. Then, compound 5 and HATU (77.9 mg, 205 μmol) were placed in an under N₂ atmosphere and stirred at rt for 1 hour. Next, the non-isolated intermediate (54.0 mg, 171 μmol) and DIPEA (89.4 μL, 512 μmol) were added and stirrer for another 2 hours. The solvents were evaporated in vacuo and the crude was redissolved in EtOAc (30 mL) and washed with water (2×20 mL) and brine (2×20 mL). The combined organic layers were dried over Na₂SO₄, filtered and the filtrate was concentrated to give a residue. The residue was purified by flash column chromatography using EtOAc as eluent to give compound 6 (85 mg, 98%) as a yellowish solid.

¹H NMR (500 MHz, DMSO-d₆, ppm) δ: 8.88 (d, J=8.1 Hz, 1H, NHCO) 7.75 (s, 1H, Ar) 7.56-7.66 (m, 2H, Ar) 7.52 (d, J=4.0 Hz, 1H, Ar) 7.38-7.42 (m, 2H, Ar) 7.34-7.37 (m, 2H, Ar) 7.26-7.32 (m, 2H, Ar) 5.17 (m, 1H, CH) 4.35 (d, J=34.9 Hz, 1H, CH) 3.85-3.94 (m, 1H, OH) 3.62 (m, 1H, CH) 3.51 (s, 3H, OCH₃) 3.11-3.19 (m, 1H, CH₂) 2.84-2.95 (m, 2H, CH₂) 2.60 (t, J=7.8 Hz, 1H, CH₂) 2.53 (t, J=7.9 Hz, 2H, CH₂) 1.78-2.07 (m, 2H, CH₂) 1.46 (d, J=7.0 Hz, 3H, CH₃).

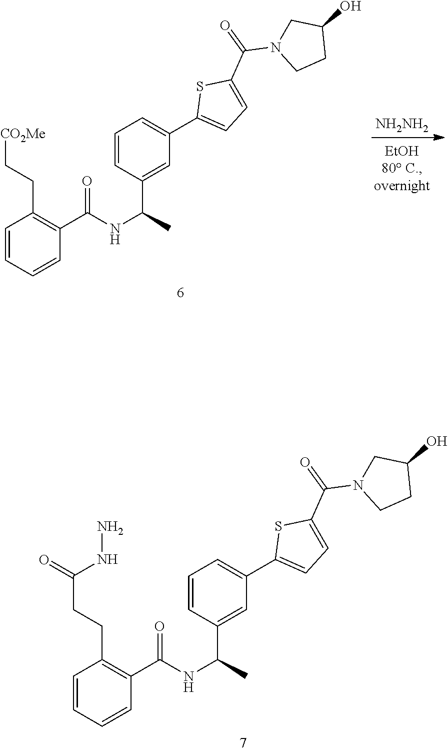 [SMILES: COC(=O)CCc1ccccc1C(=O)N[C@H](C)c1cccc(-c2ccc(C(=O)N3CC[C@H](O)C3)s2)c1 ; "NH2NH2" ; C[C@@H](NC(=O)c1ccccc1CCC(=O)NN)c1cccc(-c2ccc(C(=O)N3CC[C@H](O)C3)s2)c1]

Synthetic Procedure: To a solution of compound 6 (31.0 mg, 61.2 umol) in EtOH (1 mL) was added NH₂NH₂·H₂O (46 mg, 918 umol). The mixture was stirred at 80° C. for 12 hours. After cooling to room temperature, the reaction mixture was concentrated to give a residue. The residue was diluted with H₂O (10 mL) and the aqueous phase was extracted with EtOAc (10 mL×3). The combined organic layers were dried over Na₂SO₄, filtered and the filtrate was concentrated to give compound 7 (30.0 mg, 97%) as a colorless oil.

¹H NMR (500 MHz, Methanol-d4, ppm) δ: 7.77 (s, 1H, Ar) 7.64-7.66 (m, 1H, Ar) 7.58-7.63 (m, 1H, Ar) 7.41-7.47 (m, 3H, Ar) 7.34-7.40 (m, 2H, Ar) 7.28 (d, J=7.5 Hz, 2H, Ar) 5.26 (m, 1H, CH) 4.50 (d, J=25.2 Hz, 1H, CH) 3.95-4.04 (m, 1H, CH₂) 3.61-3.82 (m, 2H, CH₂) 3.35 (b.s, 2H, NH₂) 3.20-3.30 (m, 1H, CH₂) 2.92-3.04 (m, 2H, CH₂) 2.44 (td, J=7.7, 1.4 Hz, 2H, CH₂) 1.93-2.20 (m, 2H, CH₂) 1.59 (d, J=7.0 Hz, 3H, CH₃).

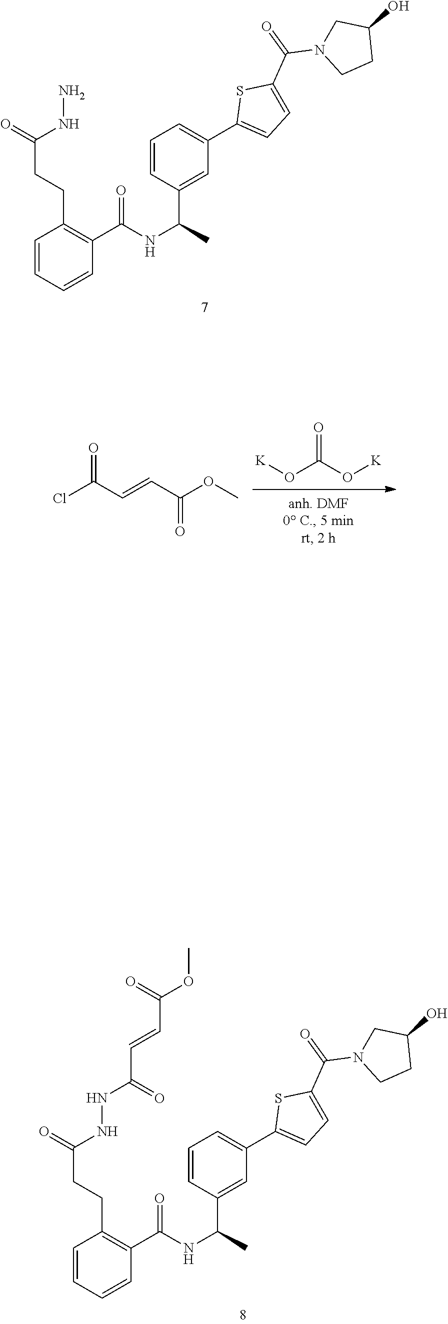

Synthetic Procedure: Compound 7 (45.0 mg, 88.8 μmol) is dissolved in anh. DMF (1.50 mL) and potassium carbonate (18.4 mg, 133 μmol) was added. The reaction mixture was purged with N₂. Then, methyl succinyl chloride (13 μL, 107 μmol) was added dropwise at 0° C. The mixture was stirred at rt for 2 h and then, diluted with 25 mL EtOAc and washed with water (3×25 mL) to remove DMF. The organic layers were combined, dried with MgSO₄, and concentrated under reduced pressure. The crude residue was purified by silica gel flash chromatography using EtOAc with 1-5% MeOH as eluent to give compound 8 (15.7 mg, 29%) as a yellowish solid.

¹H NMR (500 MHz, methanol-d4, ppm) δ: 7.77 (s, 1H, Ar), 7.65-7.61 (m, 1H, Ar), 7.61-7.56 (m, 1H, Ar), 7.46-7.41 (m, 3H, Ar), 7.40-7.35 (m, 2H, Ar), 7.35-7.31 (m, 1H, Ar), 7.27 (t, J=8.2 Hz, 1H, Ar), 7.01 (d, J=15.6 Hz, 1H, CH), 6.78 (d, J=15.6 Hz, 1H, CH), 5.26 (d, J=7.0 Hz, 1H, CH), 4.58-4.41 (m, 1H, CH), 4.05-3.93 (m, 1H, CH₂), 3.79 (s, 3H, CH₃), 3.78-3.58 (m, 3H, CH₂), 3.04 (d, J=7.6 Hz, 2H, CH₂), 2.59 (t, J=7.6 Hz, 2H, CH₂), 2.28-2.13 (m, 1H, CH₂), 2.13-1.94 (m, 1H, CH₂), 1.59 (d, J=7.0 Hz, 3H, CH₃). LCMS (5-95_4 min) Rt=2.339 min, [M+1]=619.29, 96% purity.

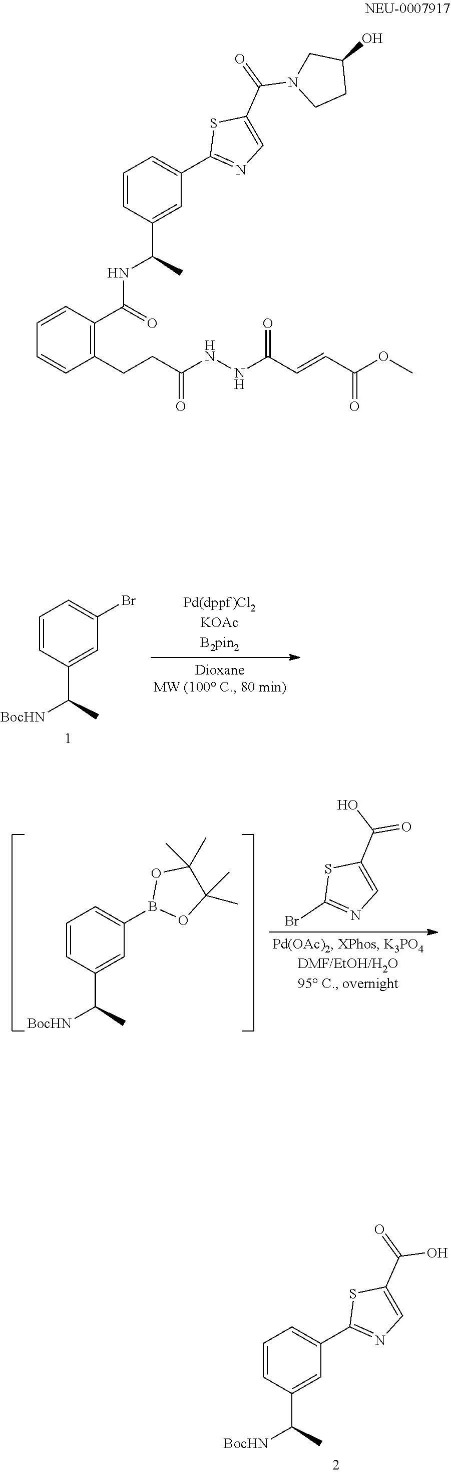

Synthetic Procedure: Compound 1 (770 mg, 2.57 mmol), potassium acetate (378 mg, 3.85 mmol), B₂pin₂ (782 mg, 3.08 mmol) and Pd(dppf)Cl₂ (105 mg, 128 μmol) were placed in a microwave vial and purged with nitrogen. Then, anh. dioxane (15.0 mL) was added and the mixture was stirred at 100° C. for 80 min in the MW. After completion, the mixture was filtrated through celite and concentrated in vacuum. The residue was used in the next step without purification.

Next, a flask fitted with a rubber septum was charged with the non-isolated intermediate (570 mg, 1.64 mmol), 2-bromothiazole-5-carboxylic acid (512 mg, 2.46 mmol), XPhos (39.1 mg, 82.1 μmol), Pd(OAc)₂ (18.4 mg, 82.1 μmol), K₃PO₄ (871 mg, 4.10 mmol) and purged with N₂. Then, degassed DMF (5.00 mL), EtOH 5.00 mL) and water (2.500 mL) were added. The mixture was stirred at 95° C. overnight. The reaction mixture was then cooled to room temperature, diluted with ethyl acetate (50 mL), filtered through celite, and concentrated in vacuo. The crude was purified by reverse phase flash column chromatography using water to methanol as eluents to give compound 2 (235 mg, 41%) as a white solid.

¹H NMR (500 MHz, DMSO-d₆, ppm) δ: 7.85 (s, 1H, Ar) 7.79 (s, 1H, Ar) 7.72 (d, J=7.5 Hz, 1H, Ar) 7.51 (d, J=8.1 Hz, 1H, NH) 7.40 (t, J=7.6 Hz, 1H, Ar) 7.33-7.37 (m, 1H, Ar) 4.60-4.72 (m, 1H, CH) 1.37 (s, 9H, CH₃ [Boc]) 1.33 (d, J=7.0 Hz, 3H, CH₃).

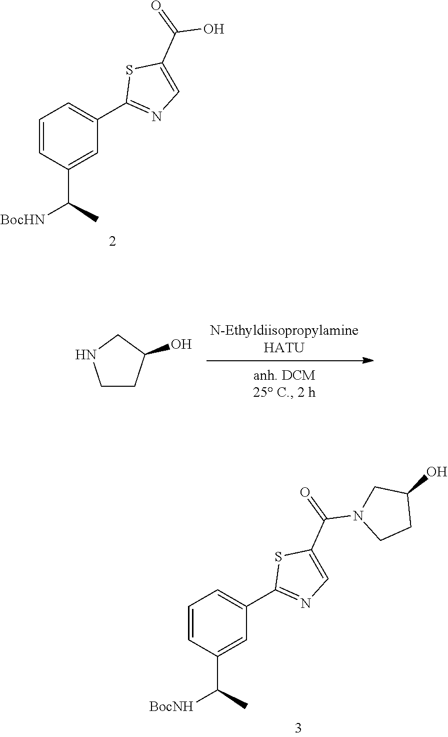 [SMILES: C[C@@H](N)c1cccc(-c2ncc(C(=O)O)s2)c1 ; O[C@H]1CCNC1 ; C[C@@H](NC(=O)OC(C)(C)C)c1cccc(-c2ncc(C(=O)N3CC[C@H](O)C3)s2)c1]

Synthetic Procedure: Compound 2 (218 mg, 626 μmol) and HATU (285 mg, 751 mol) were placed in an under N₂ atmosphere and anh. DCM (13 mL) was added. The mixture was stirred at rt for 1 hour. Then, 3-pyrrolidinol (87.3 μL, 751 μmol) and N-ethyldiisopropylamine (N-ethyldiisopropylamine (328 μL, 1.88 mmol) were added and the reaction mixture was stirred at 25° C. for 2 hours. The solvents were evaporated to give a residue that was redissolved in EtOAc (20 mL) and washed with water (2×15 mL) and brine (2×15 mL) to afford compound 3 (181 mg, 69%) as a yellowish solid.

¹H NMR (500 MHz, DMSO-d₆, ppm) δ: 8.29-8.39 (m, 1H, Ar) 7.94 (s, 1H, Ar) 7.81-7.87 (m, 1H, Ar) 7.53 (d, J=7.9 Hz, 1H, NH) 7.43-7.49 (m, 2H, Ar) 5.07 (dd, J=25.5, 3.4 Hz, 1H, OH) 4.64-4.74 (m, 1H, CH) 4.36 (d, J=36.0 Hz, 1H, CH), 3.84-3.95 (m, 2H, CH₂) 3.57-3.66 (m, 2H, CH₂) 3.43-3.57 (m, 1H, CH₂) 1.81-2.09 (m, 2H, CH₂) 1.37 (s, 9H, CH₃-[Boc]), 1.34 (d, J=7.0 Hz, 3H, CH₃).

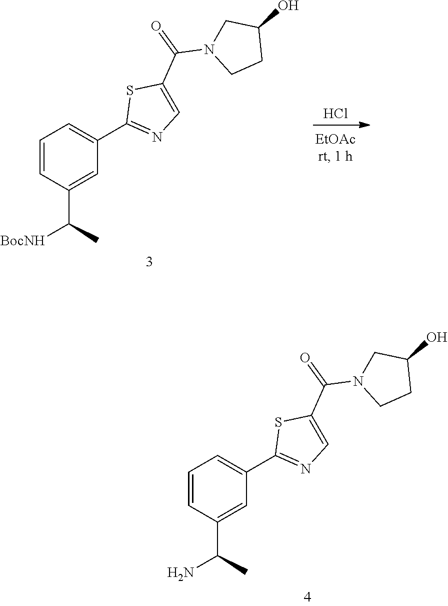 [SMILES: C[C@@H](NC(=O)OC(C)(C)C)c1cccc(-c2ncc(C(=O)N3CC[C@H](O)C3)s2)c1 ; Cl ; C[C@@H](N)c1cccc(-c2ncc(C(=O)N3CC[C@H](O)C3)s2)c1]

Synthetic Procedure: To a solution of compound 3 (281 mg, 673 μmol) in EtOAc (2 mL), a few drops of HCl were added. After the addition, a pink precipitate was formed. The mixture was stirred for 1 hour. The solid was collected by filtration, rinsed with Et₂O, and dried under reduced pressure to give compound 4 (214 mg, quant.) as a brown solid.

¹H NMR (500 MHz, DMSO-d₆, ppm) δ: 8.69 (br. s., 2H, NH₂), 8.40-8.31 (m, 1H, Ar), 8.18 (s, 1H, Ar), 7.99 (d, J=7.8 Hz, 1H, Ar), 7.70 (d, J=7.8 Hz, 1H, Ar), 7.58 (t, J=7.7 Hz, 1H, Ar), 4.83 (br. s., 1H, OH), 4.51 (td, J=6.0, 12.1 Hz, 1H, CH), 4.42-4.29 (d, J=36.0 Hz, 1H, CH), 3.96-3.82 (m, 1H, CH₂), 3.71-3.39 (m, 3H, CH₂), 2.08-1.80 (m, 2H, CH₂), 1.56 (d, J=7.0 Hz, 3H, CH₃).

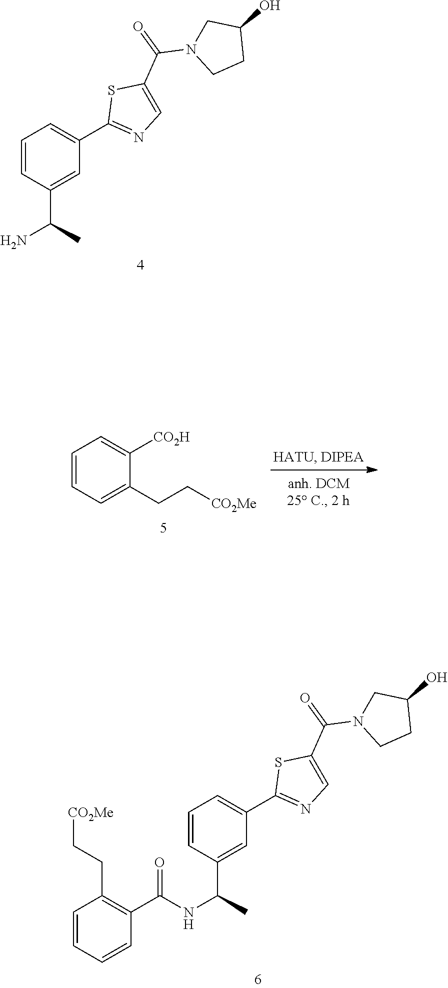 [SMILES: C[C@@H](N)c1cccc(-c2ncc(C(=O)N3CC[C@H](O)C3)s2)c1 ; COC(=O)CCc1ccccc1C(=O)O ; COC(=O)CCc1ccccc1C(=O)N[C@H](C)c1cccc(-c2ncc(C(=O)N3CC[C@H](O)C3)s2)c1]

Synthetic Procedure: Compound 5 (168 mg, 809 μmol) and HATU (308 mg, 809 μmol) were placed in an under N₂ atmosphere and dissolved in anh. DCM (2.56 mL). The mixture was stirred at rt for 1 hour. Then, compound 4 (214 mg, 674 μmol) and DIPEA (353 μL, 2.02 mmol) were added and stirred for 2 hours. The solvents were evaporated in vacuo and the crude was redissolved in EtOAc (30 mL) and washed with water (2×20 mL) and brine (2×20 mL). The combined organic layers were dried over Na₂SO₄, filtered and the filtrate was concentrated to give a residue. The residue was purified by flash column chromatography using DCM:MeOH (95:5) as eluent to give compound 6 (236 mg, 69%) as a yellowish solid.

¹H NMR (500 MHz, CDCl₃, ppm) δ: 8.21-8.10 (m, 1H, Ar), 8.04 (d, J=4.7 Hz, 1H), 7.85 (d, J=7.6 Hz, 1H, Ar), 7.53 (d, J=7.6 Hz, 1H, Ar), 7.47-7.43 (m, 1H, Ar), 7.41 (d, J=7.6 Hz, 1H, Ar), 7.34 (t, J=7.9 Hz, 1H, Ar), 7.24 (d, J=7.5 Hz, 2H, Ar), 6.99-6.93 (m, 1H, NH), 5.38 (quin, J=7.1 Hz, 1H, CH), 4.59 (d, J=17.7 Hz, 1H, CH), 3.93-3.84 (m, 1H, CH₂), 3.82-3.71 (m, 2H, CH₂), 3.61 (s, 3H, OCH₃), 3.05 (dt, J=3.2, 7.3 Hz, 2H, CH₂), 2.72 (dt, J=2.7, 7.4 Hz, 2H, CH₂), 2.18-1.97 (m, 2H, CH₂), 1.65 (d, J=7.0 Hz, 3H, CH₃).

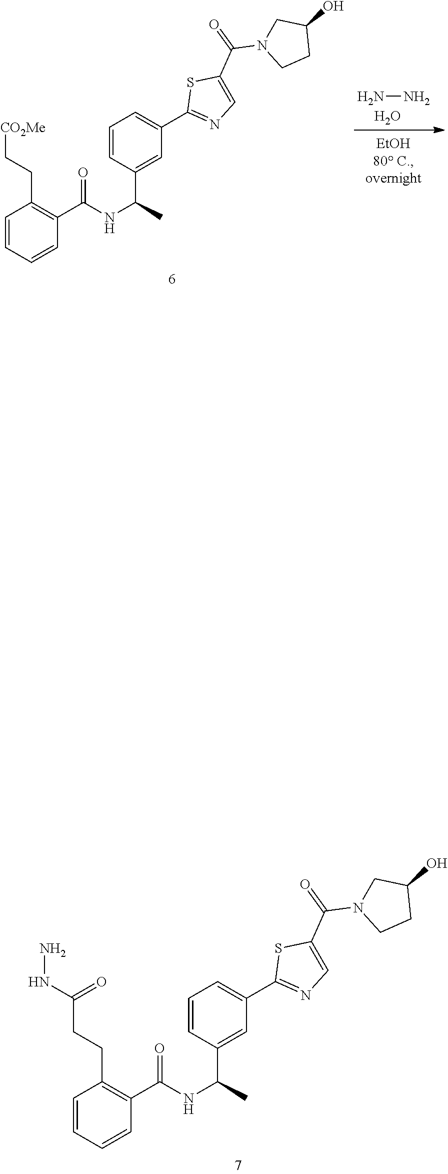

Synthetic Procedure: To a solution of compound 6 (236 mg, 465.0 μmol) in EtOH (7.50 mL) was added NH₂NH₂·H₂O (349 mg, 6.97 mmol). The mixture was stirred at 80° C. for 12 hours. After cooling to room temperature, the reaction mixture was concentrated to give a residue. The residue was diluted with H₂O (10 mL) and the aqueous phase was extracted with EtOAc (10 mL×3). The combined organic layers were dried over Na₂SO₄, filtered and the filtrate was concentrated to give compound 7 (176 mg, 74%) as a colorless oil.

¹H NMR (500 MHz, Methanol-d₄, ppm) δ: 8.29 (d, J=25.6 Hz, 1H, Ar) 8.10 (s, 1H, Ar) 7.91 (d, J=7.6 Hz, 1H, Ar) 7.60 (d, J=7.8 Hz, 1H, Ar) 7.51 (t, J=7.8 Hz, 1H, Ar) 7.38-7.41 (m, 1H, Ar) 7.34-7.37 (m, 1H, Ar) 7.26-7.31 (m, 2H, Ar) 5.29 (q, J=7.0 Hz, 1H, CH) 4.51 (d, J=26.6 Hz, 1H, CH) 3.93-4.05 (m, 1H, CH₂) 3.73-3.82 (m, 1H, CH₂) 3.67 (m, J=7.6 Hz, 1H, CH₂) 2.90-3.09 (m, 2H, CH₂) 2.44 (t, J=7.7 Hz, 2H, CH₂) 1.96-2.21 (m, 2H, CH2) 1.60 (d, J=7.0 Hz, 3H, CH₃).

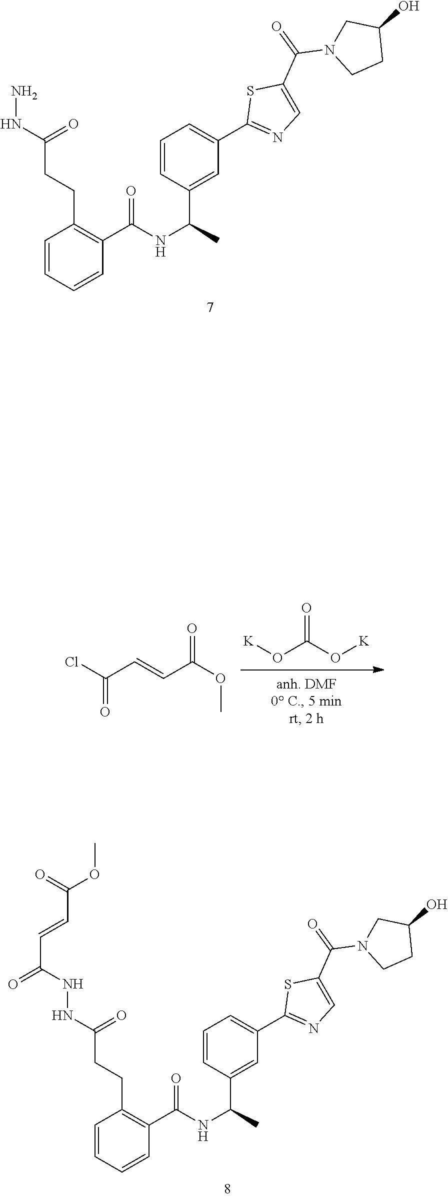

Synthetic Procedure: Compound 7 (234 mg, 460 μmol) is dissolved in anh. DMF (7.77 mL) and potassium carbonate (63.6 mg, 460 μmol) was added. The reaction mixture was purged with N₂. Then, methyl succinyl chloride (67 μL, 552 μmol) was added dropwise at 0° C. The mixture was stirred at rt for 2 h and then diluted with 25 mL EtOAc and washed with water (3×25 mL) to remove DMF. The organic layers were combined, dried with MgSO₄, and concentrated under reduced pressure. The crude residue was purified by silica gel flash chromatography using EtOAc with 1-5% MeOH as eluent to give compound 8 (28.5 mg, 10%) as a yellowish solid.

¹H NMR (500 MHz, methanol-d4, ppm) δ: 9.00 (d, J=7.6 Hz, 1H, NH) 8.31 (d, J=24.7 Hz, 1H, Ar) 8.09 (s, 1H, Ar) 7.90 (d, J=7.8 Hz, 1H, Ar) 7.60 (d, J=7.8 Hz, 1H, Ar) 7.51 (t, J=7.8 Hz, 1H, Ar) 7.32-7.41 (m, 3H, Ar) 7.30 (t, J=7.6 Hz, 1H, Ar) 7.02 (d, J=15.6 Hz, 1H, CH) 6.79 (m, 1H, CH) 5.22-5.34 (m, 1H, CH) 4.51 (d, J=26.4 Hz, 1H, CH) 3.92-4.06 (m, 1H, CH₂) 3.78-3.81 (m, 3H, CH₃) 3.76 (m, 2H, CH₂) 3.63-3.71 (m, 1H, CH₂) 2.94-3.13 (m, 2H, CH2) 2.59 (t, J=7.6 Hz, 2H, CH₂) 1.97-2.21 (m, 2H, CH₂) 1.60 (d, J=7.2 Hz, 3H, CH₃). LCMS (5-95_4 min) Rt=2.169 min, [M+1]=620.34, 97.27% purity.

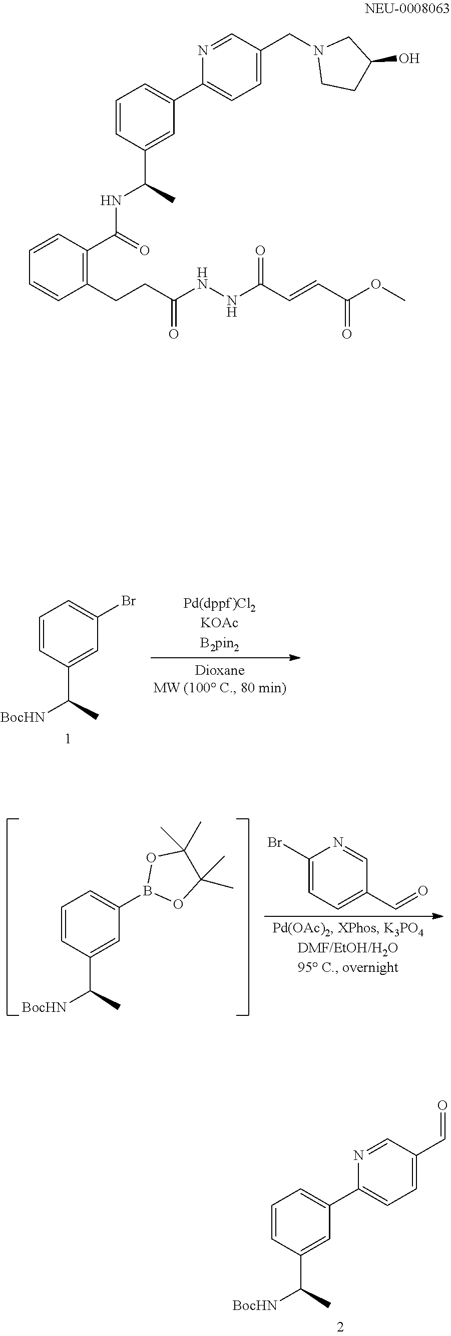

Synthetic Procedure: Compound 1 (624 mg, 2.08 mmol), potassium acetate (306 mg, 3.12 mmol), B₂pin₂ (633 mg, 2.49 mmol) and Pd(dppf)Cl₂ (84.9 mg, 104 μmol) were placed in a microwave vial and purged with nitrogen. Then, anh. dioxane (12 mL) was added and the mixture was stirred at 100° C. for 80 min in the MW. After completion, the mixture was filtrated through celite and concentrated in vacuum. The residue was used in the next step without purification.

Next, a flask fitted with a rubber septum was charged with the non-isolated intermediate (722 mg, 2.08 mmol), 6-bromo-3-pyridinecarboxaldehyde (580 mg, 3.12 mmol), XPhos (49.6 mg, 104 μmol), Pd(OAc)₂ (23.3 mg, 104 μmol), K₃PO₄ (1.10 g, 5.20 mmol) and purged with N₂. Then, degassed DMF (5.00 mL), EtOH 5.00 mL) and water (2.500 mL) were added. The mixture was stirred at 95° C. overnight. The reaction mixture was then cooled to room temperature, diluted with ethyl acetate (50 mL), filtered through celite, and concentrated in vacuo. The crude was purified by flash column chromatography using hexane to hexane:ethyl acetate (7:3) as eluents to give compound 2 (376 mg, 55%) as a white solid.

¹H NMR (500 MHz, CDCl₃, ppm) δ:10.15 (s, 1H, CHO), 9.13 (d, J=1.5 Hz, 1H, Ar), 8.24 (dd, J=2.1, 8.2 Hz, 1H, Ar), 8.06 (s, 1H, Ar), 7.95 (d, J=7.5 Hz, 1H, Ar), 7.91 (d, J=8.2 Hz, 1H, Ar), 7.52-7.47 (t, J=7.6 Hz, 1H, Ar), 7.46-7.43 (m, 1H, Ar), 4.90 (m, 1H, CH), 1.53 (d, J=6.0 Hz, 3H, CH₃), 1.43 (s., 9H, OCH₃).

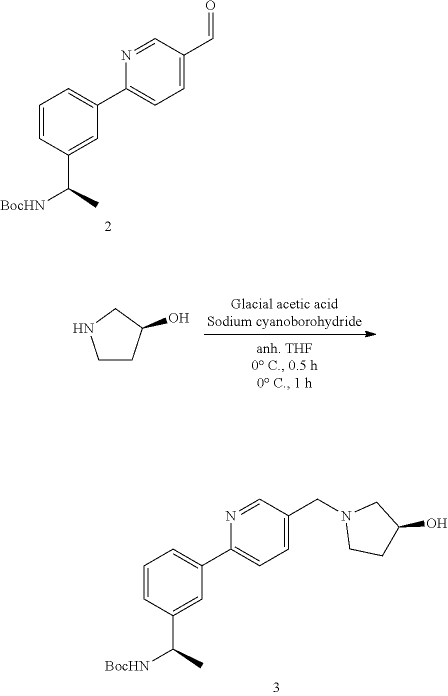 [SMILES: C[C@@H](NC(=O)OC(C)(C)C)c1cccc(-c2ccc(C=O)cn2)c1 ; O[C@H]1CCNC1 ; C[C@@H](NC(=O)OC(C)(C)C)c1cccc(-c2ccc(CN3CC[C@H](O)C3)cn2)c1]

Synthetic Procedure: To a solution of compound 2 (376 mg, 1.15 mmol) and (3S)-pyrrolidin-3-ol (134 μL, 1.15 mmol) in anh. THF (9.17 mL) was added AcOH (66.5 μL, 1.15 mmol) at 0° C. for 0.5 hours. Then NaBH₃CN (217 mg, 3.46 mmol) was added to the mixture. The mixture was stirred at 0° C. for 1 hour. The residue was diluted with H₂O (40 mL) and the aqueous phase was extracted with EtOAc (60 mL×3). The combined organic layers were dried over Na₂SO₄, filtered and the filtrate was concentrated to give a residue. The residue was purified by flash column chromatography using DCM (100%) to DCM:MeOH (85:15) as eluents to give compound 3 (156 mg, 34%).

¹H NMR (500 MHz, Methanol-d4, ppm) δ: 8.70 (d, J=1.7 Hz, 1H, Ar), 8.02-7.98 (m, 1H, Ar), 7.97-7.92 (m, 2H, Ar), 7.86 (d, J=7.5 Hz, 1H, Ar), 7.49-7.45 (m, 1H, Ar), 7.44-7.41 (m, 1H, Ar), 4.81-4.74 (m, 1H, CH), 4.54-4.47 (m, 1H, CH), 4.23 (q, J=14.2 Hz, 2H, CH₂), 3.25-3.17 (m, 1H, CH₂), 3.15-3.09 (m, 1H, CH₂), 3.06 (m, 1H, CH₂), 2.36-2.21 (m, 1H, CH₂), 1.96 (m, 1H, CH₂), 1.45 (d, J=7.0 Hz, 3H, CH₃), 1.43 (br. s., 9H, CH₃-[Boc]).

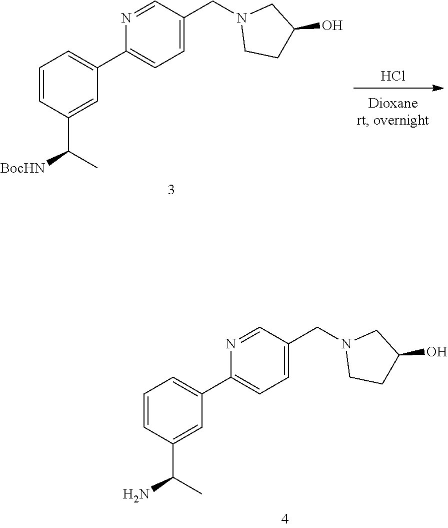

Synthetic Procedure: To a solution of compound 3 (190 mg, 478 μmol) in dioxane (1 mL), a few drops of HCl were added. After the addition, a pink precipitate was formed. The mixture was stirred overnight. The solid was collected by filtration, rinsed with Et₂O, and dried under reduced pressure to give compound 4 (142 mg, quant.) as a yellowish solid.

¹H NMR (500 MHz, methanol-d4, ppm) δ: 9.2 (d, J=8.7 Hz, 1H, Ar), 8.96-8.86 (m, 1H, Ar), 8.54 (d, J=8.4 Hz, 1H, Ar,), 8.27-8.19 (m, 1H, Ar), 8.08 (d, J=8.2 Hz, 1H, Ar), 7.89-7.83 (m, 1H, Ar), 7.83-7.75 (m, 1H, Ar), 4.87-4.74 (m, 2H, CH+CH₂), 4.74-4.57 (m, 2H, CH+CH₂), 3.93-3.68 (m, 2H, CH₂), 3.60-3.51 (m, 1H, CH₂), 3.50-3.40 (m, 1H, CH₂), 2.58-2.43 (m, 1H, CH₂), 2.26-2.00 (m, 1H, CH₂), 1.76 (d, J=6.9 Hz, 3H, CH₃).

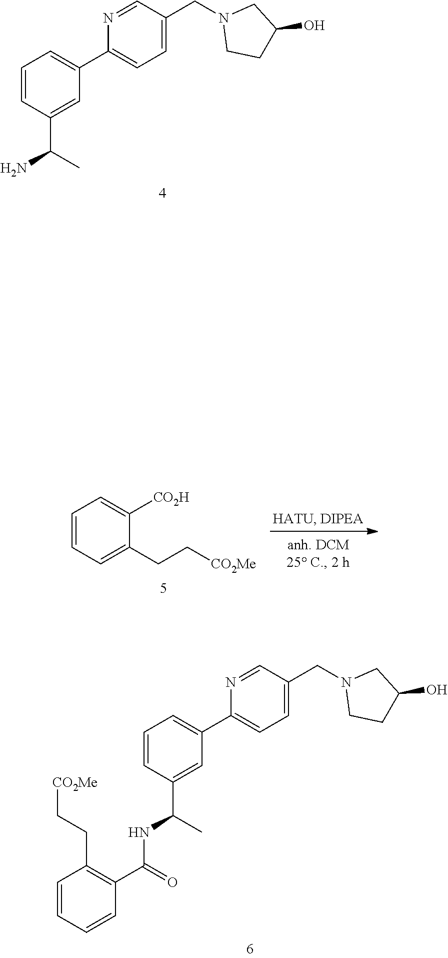 [SMILES: C[C@@H](N)c1cccc(-c2ccc(CN3CC[C@H](O)C3)cn2)c1 ; COC(=O)CCc1ccccc1C(=O)O ; COC(=O)CCc1ccccc1C(=O)N[C@H](C)c1cccc(-c2ccc(CN3CC[C@H](O)C3)cn2)c1]

Synthetic Procedure: Compound 5 (118 mg, 398 μmol) and HATU (182 mg, 477 mol) were placed in an under N₂ atmosphere and dissolved in anh. DCM (5 mL). The mixture was stirred at rt for 1 hour. Then, compound 4 (99.4 mg, 477 μmol) and DIPEA (208 μL, 1.19 mmol) were added and stirred for 2 hours. The solvents were evaporated in vacuo and the crude was redissolved in EtOAc (30 mL) and washed with water (2×20 mL) and brine (2×20 mL). The combined organic layers were dried over Na₂SO₄, filtered and the filtrate was concentrated to give a residue. The residue was purified by flash column chromatography using DCM:MeOH (80:20) as eluent to give compound 6 (130 mg, 67%) as a yellowish solid.

¹H NMR (500 MHz, methanol-d4, ppm) δ: 8.94 (d, J=7.8 Hz, 1H, NHCH), 8.77 (d, J=1.8 Hz, 1H, Ar), 8.14-8.10 (m, 1H, Ar), 8.07-7.97 (m, 2H, Ar), 7.94 (d, J=7.6 Hz, 1H, Ar), 7.58-7.54 (m, 1H, Ar), 7.51 (t, J=7.8 Hz, 1H, Ar), 7.41-7.34 (m, 2H, Ar), 7.28 (d, J=7.5 Hz, 2H, Ar), 5.34-5.24 (m, 1H, CH), 4.61-4.57 (m, 1H, CH), 4.55-4.46 (m, 2H, CH₂), 3.67-3.59 (m, 1H, CH₂), 3.56 (s, 3H, OCH₃), 3.50-3.39 (m, 2H, CH₂), 3.04-2.94 (m, 3H, CH₂), 2.56 (t, J=7.9 Hz, 2H, CH₂), 2.41-2.26 (m, 1H, CH₂), 2.13-2.02 (m, 1H, CH₂), 1.61 (d, J=7.0 Hz, 3H, CH₃)

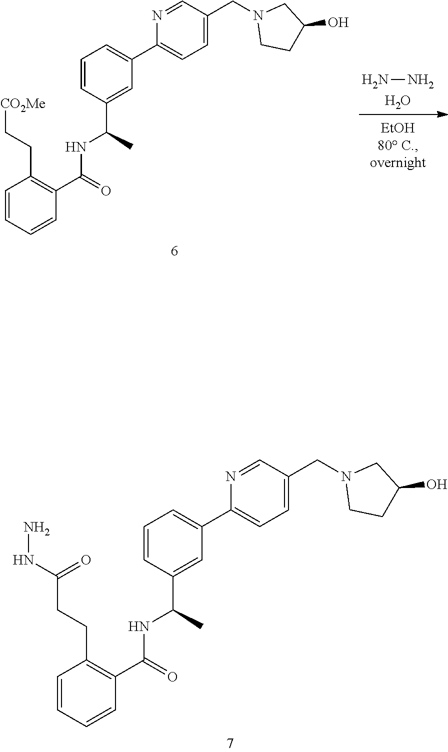

Synthetic Procedure: To a solution of compound 6 (130 mg, 266 μmol) in EtOH (4.30 mL) was added NH₂NH₂·H₂O (200 mg, 3.99 mmol). The mixture was stirred at 80° C. for 12 hours. After cooling to room temperature, the reaction mixture was concentrated to give a residue. The residue was diluted with H₂O (10 mL) and the aqueous phase was extracted with EtOAc (10 mL×3). The combined organic layers were dried over Na₂SO₄, filtered and the filtrate was concentrated to give compound 7 (128 mg, 99%) as a colorless oil.

¹H NMR (500 MHz, methanol-d4, ppm) δ: 8.59 (d, J=1.5 Hz, 1H, Ar), 8.04 (s, 1H, Ar), 7.90 (s, 1H, Ar), 7.87-7.82 (m, 2H, Ar), 7.54-7.46 (m, 2H, Ar), 7.37 (s, 2H, Ar), 7.27 (d, J=7.5 Hz, 2H), 5.35-5.27 (m, 1H, CH), 4.40-4.33 (m, 1H, CH), 3.75 (d, J=18.0 Hz, 2H, CH₂), 2.98 (d, J=7.6 Hz, 2H, CH₂), 2.82 (d, J=6.0 Hz, 2H, CH₂), 2.65-2.52 (m, 2H, CH₂), 2.42 (t, J=7.0 Hz, 2H, CH₂), 2.22-2.12 (m, 1H, CH₂), 1.80-1.70 (m, 1H, CH₂), 1.62 (d, J=7.0 Hz, 3H, CH₃).

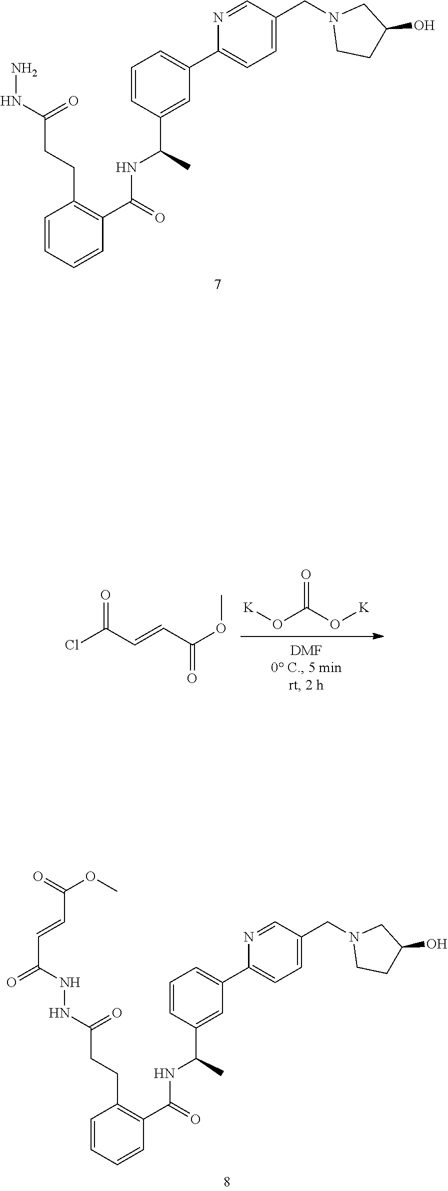

Synthetic Procedure: Compound 7 (96.3 mg, 198 μmol) is dissolved in anh. DMF (3.34 mL) and potassium carbonate (27.3 mg, 198 μmol) was added. The reaction mixture was purged with N₂. Then, methyl succinyl chloride (28.8 μL, 237 μmol) was added dropwise at 0° C. The mixture was stirred at rt 5 h and then diluted with 25 mL EtOAc and washed with water (3×25 mL) to remove DMF. The organic layers were combined, dried with MgSO₄, and concentrated under reduced pressure. The crude residue was purified by silica gel flash chromatography using DCM with 1-15% MeOH as eluent to give compound 8 (19 mg, 16%) as a off-white solid.

¹H NMR (500 MHz, methanol-d4, ppm) δ: 8.72-8.68 (m, 1H, Ar), 8.08 (d, J=1.9 Hz, 1H, Ar), 8.00 (dd, J=8.2, 2.3 Hz, 1H, Ar), 7.94 (dd, J=8.2, 0.9 Hz, 1H, Ar), 7.89 (dt, J=7.5, 1.6 Hz, 1H, Ar), 7.57-7.48 (m, 2H, Ar), 7.38 (td, J=7.3, 1.3 Hz, 2H, Ar), 7.35-7.31 (m, 1H, Ar), 7.30-7.25 (m, 1H, Ar), 7.01 (d, J=15.6 Hz, 1H, CH), 6.79 (d, J=15.6 Hz, 1H, CH), 5.31 (q, J=7.0 Hz, 1H, CH), 4.49 (dp, J=7.6, 2.4 Hz, 1H, CH), 4.18 (q, J=13.1 Hz, 2H, CH₂), 3.79 (s, 3H, OCH₃), 3.27 (d, J=8.8 Hz, 1H, CH₂), 3.21-3.14 (m, 1H, CH₂), 3.02 (qd, J=12.9, 5.5 Hz, 3H, CH₂+CH₂), 2.57 (t, J=8.0 Hz, 2H, CH₂), 2.26 (dtd, J=14.3, 8.3, 6.2 Hz, 1H, CH₂), 1.96-1.89 (m, 1H, CH₂), 1.61 (d, J=7.0 Hz, 3H, CH₃). LCMS (5-95_4 min) Rt=1.822 min, [M+1]=600.33, 99.79% purity.

While there have been shown and described what are at present considered the preferred embodiments of the invention, those skilled in the art may make various changes and modifications which remain within the scope of the invention defined by the appended claims.

What is claimed is:

1. A compound having the following structure: 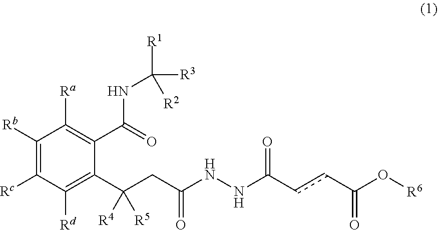
wherein:
R¹ is a hydrocarbon group containing at least one aromatic or heteroaromatic ring or fused ring system, wherein the at least one aromatic or heteroaromatic ring or fused ring system is optionally substituted with one or more groups selected from halogen atoms, hydrocarbon groups containing 1-6 carbon atoms; OR′ groups; C(O)OR′ groups; amine groups of the formula NR′₂; and amide groups of the formula NHC(O)R′ or C(O)NR′₂, wherein R′ is independently selected from H and hydrocarbon groups containing 1-6 carbon atoms;
R² and R³ are independently selected from the group consisting of hydrogen (H), halogen atom, and hydrocarbon groups containing 1-6 carbon atoms optionally substituted with one or more halogen atoms and optionally containing an —O— or —NH— linkage, wherein R² and R³ are optionally interconnected to form a three-, four-, five-, or six-membered cycloalkyl or heterocycloalkyl ring;
R⁴ and R⁵ are independently selected from the group consisting of H, halogen atom, and hydrocarbon groups containing 1-6 carbon atoms optionally substituted with one or more halogen atoms and optionally containing an —O— or —NH— linkage, wherein R⁴ and R⁵ are optionally interconnected to form a three-, four-, five-, or six-membered cycloalkyl or heterocycloalkyl ring;
R⁶ is H or a hydrocarbon group containing 1-12 carbon atoms and optionally containing one or more heteroatoms selected from O, N, S, and F;
R^{a}, R^{b}, R^{c}, and R^{d} are independently selected from the group consisting of H; halogen atom; hydrocarbon groups containing 1-3 carbon atoms; amine group of the formula NR′₂; amide groups of the formula NHC(O)R′ or C(O)NHR′; carbamate groups of the formula NHC(O)OR′; urea groups of the formula NHC(O)NR′₂; and —(CH₂)ₚ—T, wherein T is a hydrocarbon group containing at least one —NH— linkage; R′ is independently selected from H and hydrocarbon groups containing 1-6 carbon atoms; and p is an integer of 0-3;
the dashed line represents an optional double bond;
and pharmaceutically acceptable salts thereof.

2. The compound of claim 1, wherein R¹ is not naphthyl.

3. The compound of claim 1, wherein the compound is an enantiomerically enriched or isolated version having the following formula in which R³ is H and thus not shown: 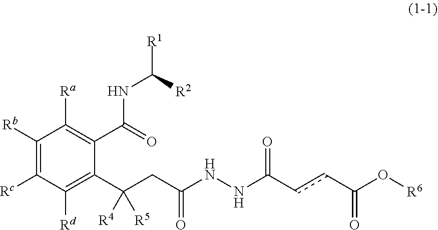
wherein R² is a halogen atom or a hydrocarbon group containing 1-6 carbon atoms optionally substituted with one or more halogen atoms and optionally containing an —O— or —NH— linkage; and R¹, R⁴, R⁵, R⁶, R^{a}, R^{b}, R^{c}, and R^{d} are as defined in claim 1.

4. The compound of claim 3, wherein R¹ is not naphthyl.

5. The compound of claim 1, wherein the compound has the following formula: 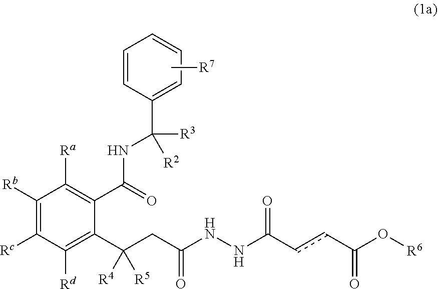
wherein R², R³, R⁴, R⁵, R⁶, R^{a}, R^{b}, R^{c}, and R^{d} are as defined in claim 1; and
R⁷ comprises a heterocyclic ring or ring system, any of which may be a substituent of or fused to the shown phenyl ring to which R⁷ is attached.

6. The compound of claim 5, wherein the compound is an enantiomerically enriched or isolated version having the following formula in which R³ is H and thus not shown: 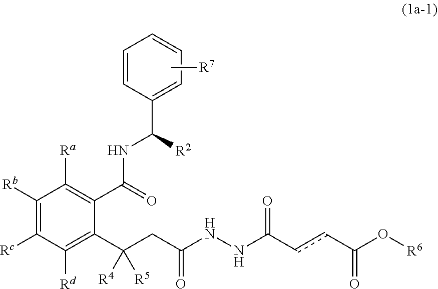
wherein R² is a halogen atom or a hydrocarbon group containing 1-6 carbon atoms optionally substituted with one or more halogen atoms and optionally containing an —O— or —NH— linkage; and R⁴, R⁵, R⁶, R^{a}, R^{b}, R^{c}, and R^{d} are as defined in claim 1.

7. The compound of claim 5, wherein R⁷ is a five-membered or six-membered heteroaromatic ring or fused ring system containing at least one ring nitrogen atom.

8. The compound of claim 5, wherein R⁷ is a thiazole ring.

9. The compound of claim 1, wherein R² and R³ are interconnected to form a three-, four-, five-, or six-membered cycloalkyl or heterocycloalkyl ring.

10. The compound of claim 9, wherein the compound has the following formula: 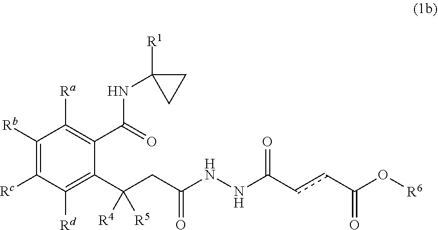
wherein R¹, R⁴, R⁵, R⁶, R^{a}, R^{b}, R^{c}, and R^{d} are as defined in claim 1.

11. The compound of claim 9, wherein the compound has the following formula: 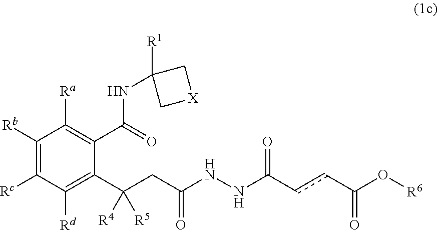
wherein R¹, R⁴, R⁵, R⁶, R^{a}, R^{b}, R^{c}, and R^{d} are as defined in claim 1; and
X is selected from the group consisting of CR^{e}R^{f}, O, S, and NR^{g}, wherein R^{e}, R^{f}, and R^{g} are independently selected from H and hydrocarbon groups containing 1-3 carbon atoms.

12. The compound of claim 1, wherein at least one of R⁴ and R⁵ is a halogen atom or a hydrocarbon group containing 1-6 carbon atoms optionally substituted with one or more halogen atoms and optionally containing an —O— or —NH— linkage, wherein R⁴ and R⁵ are optionally interconnected to form a three-, four-, five-, or six-membered cycloalkyl or heterocycloalkyl ring.

13. The compound of claim 12, wherein at least one of R⁴ and R⁵ is a halogen atom.

14. The compound of claim 12, wherein R⁴ and R⁵ are halogen atoms.

15. The compound of claim 14, wherein the compound has the following formula: 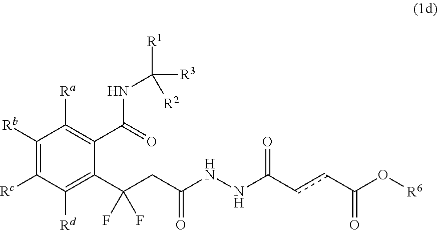
wherein R¹, R², R³, R⁶, R^{a}, R^{b}, R^{c}, and R^{d} are as defined in claim 1.

16. The compound of claim 15, wherein R¹ is not naphthyl.

17. A pharmaceutical composition comprising a compound of Formula (1) and a pharmaceutically acceptable carrier.

18. A method of inhibiting papain-like protease (PLpro) activity in a subject, the method comprising administering a therapeutically effective dosage of a compound of Formula (1) to the subject to result in inhibition of PLpro activity in the subject.

19. The method of claim 18, wherein the PLpro activity is mediated by a coronavirus PLpro.

20. A method of treating coronavirus infection in a subject, the method comprising administering a therapeutically effective dosage of a compound of Formula (1) to the subject to result in inhibition or prevention of one or more coronavirus symptoms in the subject.
